(19) United States
(12) Patent Application Publication
(10) Pub. No.: US 2011/0143351 A1
(43) Pub. Date: Jun. 16, 2011

(54) GLYOSYLATION MARKERS FOR CANCER AND CHRONIC INFLAMMATION

(75) Inventors: Pauline Rudd, Dublin (IE); James Arnold, Cambridge (GB); Radka Saldova, Dublin (IE); Louise Royle, Oxfordshire (GB); Umi Marshida Abd Hamid, Oxford (GB); Raymond Dwek, Oxford (GB); Rosa Peracaula, Girona (ES); Rafael De Llorens, Girona (ES)

(73) Assignee: NATIONAL INSTITUTE FOR BIOPR0ESSING RESEARCH AND TRAINING LIMITED

(21) Appl. No.: 12/452,720

(22) PCT Filed: Jul. 21, 2008
(86) PCT No.: PCT/GB2008/050607

§ 371 Date: Sep. 24, 2010

(30) Foreign Application Priority Data
Jul. 20, 2007 (IE) 2007/0529
Jul. 20, 2007 (US) 60951003

(51) Int. Cl. G01N 33/53; G01N 33/559; G01N 27/447; H01J 49/26

(52) U.S. Cl. 435/6.12; 435/7.21; 435/7.92; 436/501; 204/461; 250/282

(57) ABSTRACT

The present invention provides novel biomarkers for use in the diagnosis and prognosis of cancerous and malignant conditions and further of diseases which are mediated by a proinflammatory immune response. The biomarkers are glycoproteins, the levels of expression of which have been correlated by the inventors to correspond to particular disease conditions. The invention further extends to methods for use in monitoring the response to therapy of a treatment for use in the treatment of a cancerous condition or proinflammatory disease.

### FIELD OF THE INVENTION

The present invention relates to methods of diagnosing and monitoring cancer and chronic inflammation, and of monitoring the response to treatments for cancer and chronic inflammation, that involve the analysis of glycosylation. In particular, the present invention provides glycosylation markers for use in the diagnosis of cancerous and malignant conditions.

### BACKGROUND TO THE INVENTION

Detection of cancer at an early stage can improve the likelihood of survival. Many cancers can be treated and cured if they are diagnosed while tumours are still localized. However, most cancers are not detected until after they have invaded the surrounding tissue or metastasized to distant sites. For example, only 50% of breast cancers, 56% of prostate cancers and 35% of colorectal cancers are still localized at the time of diagnosis. The situation is even worse for other types of cancer. About 80% of pancreatic cancers are already metastatic at the time of diagnosis, which results in a 1 year survival rate after diagnosis of about 19% and a 5 year survival rate of about 4%. Similar 5 year survival rates (<5%) were reported for hepatocellular carcinoma. This delay in cancer detection results in a poor prognosis as early detection is important for successful treatment. As therapeutic options for cancer treatment increase, early detection of cancer becomes increasingly important for improving prognosis.

In recent years, serum protein markers have been developed for certain types of cancer. For example, prostate specific antigen (PSA), a glycoprotein secreted by prostate cells that is found in serum in prostate pathologies, is currently used as a tumour marker for prostate cancer. Other protein markers for cancer diagnostics and monitoring are alpha-fetoprotein for hepatocellular carcinoma and testicular cancer, NMP22 for bladder cancer, catecholamines for neuroblastoma, immunoglobulins for multiple myeloma, carcinoembryonic antigen (CEA) for colorectal cancer, HER-2, CA 15-3 and CA 27-29 for breast cancer, CA 125 for ovarian cancer and CA19-9 for pancreatic cancer.

Although the development of these markers facilitated the clinical management of certain types of cancer, the assays of these biomarkers are neither sensitive nor specific enough for use as the sole screening method for cancer diagnostics. Thus, it is highly desirable to develop new cancer-related biomarkers and diagnostic methodologies that will be more sensitive and more specific to detect recurrence and metastases at the earliest stages for both diagnosing and monitoring cancer progression.

Many of the serum acute phase proteins, such as, haptoglobin beta chain, α1-acid glycoprotein and α1-anti-chymotrypsin, are glycoproteins. During inflammation, the serum levels of the acute phase proteins can increase by as much as 1000 fold. The glycan structures attached to these molecules alter during long-term (chronic) inflammation. Two of the best understood glycosylation changes are the degree of glycan branching (dictated by the number of GlcNAcs attached to the chitobiose core) and the levels of Sialyl Lewis x (SLe^{x}/CD15s) structures. The SLe^{x} epitope consists of a sialic acid α2,3 linked to galactose with fucose α1,3 linked to GlcNAc, and has been implicated in leukocyte extravasation. SLe^{x} is the ligand for endothelial-selectin (E-selectin) which is exclusively expressed on endothelial cells in response to IL1-β, TNF-α, lipopolysachamide and phorbol myristate acetate. Leukocytes, which naturally express SLe^{x} epitopes, use this interaction to adhere to the endothelium and, following integrin interactions, the cells extravasate from the blood stream. SLe^{x} levels are significantly higher on metastatic cancer cells, and can be exploited by cancer cells to aid metastasis. SLe^{x} epitopes are present on the N-linked glycans attached to the acute phase proteins haptoglobin, α1-acid glycoprotein and α1-antichymotrypsin.

The N-linked glycans of α1-acid glycoprotein secreted from the HuH0-7 hepatic cell line when stimulated with the pro-inflammatory cytokines IL1-β and IL-6 show increased branching and SLe^{x} epitopes. During acute inflammation, α1-acid glycoprotein contains increased bi-antennary structures, but this shifts to an increase in tri- and tetra-antennary structures with chronic inflammation. These glycosylation changes have been associated with pregnancy, rheumatoid arthritis, chronic liver cirrhosis and chronic inflammation in cancer.

Cytokines are signalling molecules secreted by activated cells that modulate cell growth and differentiation, random and directional migration of leukocytes, inflammation and adaptive immune functions by acting in cross-modulation to elicit refined immune responses. Many tumours possess an inflammatory component, especially in the late stages of tumour development, and the inflammatory processes can promote tumour growth and progression. Inflammatory-associated cytokines include IL-β, IL1-β, TNF-α, IFN-γ TNF-β and possibly IL-8 and IL-11. In the serum IL-1, IL-β, TNF-α and LIF can stimulate liver hepatocytes to secrete acute phase proteins, in a process known as the acute phase response.

In cancer, the presence of a tumour can cause chronic inflammation and, in 90% of patients, post-surgical levels of SLe^{x} decreased with 60% reaching normal levels. High levels of SLe^{x} on serum glycoproteins, or expression of SLe^{x} epitopes on tumour tissue, is associated with poor outcome. SLe^{x} is a good prognostic factor for tumour stage (71%), but a weak diagnostic marker for non small cell lung cancer (24%).

Breast cancer is the most prevalent cancer in the world and accounts for the highest number of cancer-related deaths among women worldwide.

Each year, more than 1.1 million new cases are diagnosed with over 400,000 deaths having been recorded. As in any other malignancy, there is an urgent need for non-invasive marker(s) not only to screen, detect, diagnose, evaluate prognosis, monitor treatment and predict recurrence, but also to play a critical role in the clinical management of breast cancer patients. The most commonly used markers for breast cancer are CA 15-3 and carcinoembryonic antigen (CEA). CA 15-3, however, lacks two important criteria for a biomarker, namely specificity and sensitivity. Therefore, it is often measured together with CEA and only recommended for determining prognosis and monitoring patients (reviewed in Duffy M. J.).

The glycosylation of breast cancer has been studied for more than two decades and encompasses various aspects of the glycosylation pathway. As noted above, among the most extensively studied glycans is sialyl Lewis x (SLe^{x}/CD15s), the non-sialylated form of Lex is also known as CD15. The conformational structure of SLe^{x} and its binding to the lectin domain of E-selectin via the fucose, galactose and carboxyl group of the sialic acid is the basis of the development of glycomimetic drugs to inhibit cancer cell metastasis via E-selectin binding.

Immunohistochemistry of breast cancer tissue indicated that SLe^{x} expression was an independent prognostic indicator of survival regardless of the size of the primary tumour and lymph node involvement. A study comparing breast cancer lesions with normal breast tissue in the same patient showed that SLe^{x} expression on epithelial cells was exclusive to cancerous samples. Concurrently, both P- and E-selectin expression were significantly enhanced on endothelial cells of malignant tissue, consistent with the proposal that SLe^{x} binding to selectins aids cancer cell metastasis. It was reported that the high metastatic potential of the RCN H4 colon cancer cells to the liver is due to the expression of cell surface SLe^{x} which reduces susceptibility to hepatic sinusoidal lymphocyte-mediated killing. Overexpression of SLe^{x} on tumour cell surface glycoproteins, however, could have the reverse effect, leading to cytolysis by natural killer (NK) cells via CD94 receptor complex as well as NKG2D, NKG2C and CD161 (Higai et al., 2006) that recognizes the SLe^{x}. These reports highlighted the fact that various levels of SLe^{x} expression can lead to different biological consequences.

Ovarian cancer is the most lethal of all gynaecological cancers among women according to UK cancer mortality statistics. Most patients are diagnosed when the disease is in an advanced stage. The earlier the cancer is diagnosed, the higher the 5-year survival rate, which is more than 90% for early stage but in advanced stages III and IV decreases to 30%.

In human carcinomas, changes in glycosylation have been described including the presence of sialyl Lewis x (SLe^{x}). As discussed above, the SLe^{x} epitope consists of a GlcNAc residue with an α1,3-linked fucose as well as a β1-4-linked galactose which has an α2,3-linked sialic acid. In addition to a proposed role in tumour metastasis, SLe^{x} is also upregulated during chronic inflammation on haptoglobin, α1-acid glycoprotein and a1-antichymotrypsin and in neutrophils. Previous reports in ovarian cancer have indicated that there is a change of glycosylation on haptoglobin and IgG in ovarian cancer patients.

Several potential markers are currently being investigated including OVX1, M-CSF, inhibin, kallikreins, TPS and lysophosphatidic acid. Increasingly proteomics-based approaches in several studies are illustrating the potential for ovarian cancer biomarkers.

Currently, ovarian cancer is most frequently diagnosed by ultrasonography and the serum marker CA125. CA125 is currently the best marker for ovarian cancer, but this marker is not reliable for diagnosing early stage cancers. CA125 is elevated in 80-90% of ovarian cancer patients; the level rising with the stage of the disease. In addition, it is also higher in nonmucinous tumours than mucinous ones. CA125 can give a false positive response in benign conditions, pregnant women and other cancers. Essentially this illustrates that additional markers are needed for this lethal cancer which would replace or complement the use of CA125.

The inventors have, following extensive experimentation, surprisingly identified that by monitoring more than one change in glycosylation that is associated with the development and progression of cancerous or malignant conditions one can arrive at sensitive and specific diagnostic methodologies.

### SUMMARY OF THE INVENTION

According to a first aspect of the present invention, there is provided a method for the diagnosis of a cancerous and/or malignant condition, the method comprising the steps of:—
providing a test sample from a subject,
determining a level in the test sample of two or more glycosylation markers for a cancerous and/or malignant condition,
providing a diagnosis based on the determined level of the two or more gylcosylation markers.

Typically the two or more glycosylation markers are specific for a cancerous and/or malignant condition. Typically the diagnosis is based on comparing the determined level of the two or more glycosylation markers to a pre-determined standard scale, such that the value of the determined level can be used to determine whether the level of the two or more glycosylation markers is statistically significant.

The inventors have further identified that, in addition to the diagnosis of a cancerous and/or malignant condition, the methods and markers of the present invention have further utility in relation to methods for the prognosis of a cancerous condition in a subject.

As such, according to a second aspect of the present invention there is provided a method for the prognosis of a cancerous or malignant condition in a subject, the method comprising the steps of:
providing a test sample from a subject,
determining a level in the test sample of two or more glycosylation markers for a cancerous and/or malignant condition, and
determining the prognosis from the level of the two or more markers.

Typically the prognosis is made by comparing the determined value of the at least two glycosylation markers to known standard values or a standard curve.

Furthermore, the markers and methods of the present invention have utility in methods for monitoring the response by a subject to the treatment of a cancerous or malignant condition in a subject,

As such, according to a third aspect of the present invention there is provided a method for determining the response to therapy of a subject whom has been administered a therapeutic compound for the treatment of a cancerous and/or malignant condition, the method comprising the steps of:
providing a test sample from a subject,
determining a level in the test sample of two or more glycosylation markers for a cancerous and/or malignant condition, and
determining the response from the level of the two or more markers

Using more than one glycosylation marker in the methods of the invention has been found to provide an improved (e.g. more specific and/or sensitive) method of diagnosing, of prognosing and/or of determining the response to a therapy than can be achieved by methods that are concerned with only a single glycosylation marker. The level of improvement may be additive, but is preferably synergistic.

It is preferred that the aforementioned aspects of the present invention determine a level in the test sample of (and so determine the diagnosis, prognosis or response on the basis of) 2, 3, 4, 5, 6, 7, 8, 9, 10 or more glycosylation markers for a cancerous and/or malignant condition, preferably 5 to 10 glycosylation markers. Methods that involve the determination on the basis of all (i.e. total or unpurified) glycosylation markers in a biological sample are however not preferred. Indeed, it is preferred that the determination is carried out on less than 30, 40, 20 or 15 glycosylation markers for a cancerous and/or malignant condition.

The glycosylation markers used in the above discussed methods may be any glycosylation markers that would be known to the skilled person and/or described in the present specification, and that represent a change in the glycosylation of a glycoprotein that is associated with the development and/or progression of a cancerous and/or malignant condition. Not wishing to be restricted further, but in the interests of clarity, these markers may be selected from the group consisting of changes in glycan branching; changes in levels of oligomannose, hybrid and complex type N-glycans, O-glycans or components thereof (e.g. fucosylation, SLe^{x} epitopes, lactosamine extensions); changes in ratios of levels between glycans; GU values; or the like; or any combination thereof. The glycosylation markers may be associated with O- and/or N-linked glycans. More specifically, for example, suitable markers may be selected from the group consisting of: glycans with GU values greater than 10.65, SLe^{x} structures, A2FG1 derived from digestion of SLe^{x}, A3FG1 derived from digestion of SLe^{x}, A4FG1 derived from digestion of SLe^{x}, sialylated tri-antennary, sialylated tetra-antennary glycans, glycans containing α1,3 fucose, α1,3 monofucosylated tri-antennary glycans, α1,3 difucosylated tri-antennary glycans, α1,3 monofucosylated tetra-antennary glycans, α1,3 difucosylated tetra-antennary glycans, tetra-antennary glycans with lactosamine extensions, ratio of α2,3 sialylated glycans to α2,6 sialylated glycans, agalactosylated fucosylated biantennary glycans, core fucosylated agalactosylated biantennary glycans, core fucosylated monosialylated glycans on transferrin, SLe^{x} on glycans on haptoglobin β-chain, A3FG1 derived from digestion of SLe^{x} on glycans on haptoglobin β-chain, A4FG1 derived from digestion of SLe^{x} on glycans on haptoglobin β-chain, SLe^{x} on glycans on α1-acid glycoprotein, A3FG1 derived from digestion of SLe^{x} on glycans on α1-acid glycoprotein, SLe^{x} on glycans on α1-antichymotrypsin, A3FG1 derived from digestion of SLe^{x} on glycans on α1-antichymotrypsin, tetra-antennary tetragalactosylated glycans on α1-antitrypsin, core fucosylated agalactosylated biantennary glycans on IgG, agalactosylated glycans on IgG, sialylation on glycans on IgG, galactosylation on glycans on IgG, FA2G2S1 on glycans on transferrin, FA2BG2S1 on glycans on transferrin and A4G4 on glycans on α1-antitrypsin, or the like, or any combination thereof.

In certain embodiments, non-parametric statistical tests may be used with Kruskal Wallis test for comparison of all groups for SLe^{x} levels and subsequent Mann Whitney tests for comparison of individual groups of markers. Correlation analysis may typically be carried out using two-tailed Spearman test. In certain embodiments, a P<0.05 value may be taken as the cut-off level for significance.

Glycans on which glycosylation markers are found may be analysed as whole glycans or as digested glycans (and so on the basis of fragments of glycans).

Preferably, the markers are selected from the group consisting of: glycans with GU values greater than 10.65, SLe^{x} structures, A3FG1 derived from digestion of SLe^{x}, sialylated tri-antennary glycans, sialylated tetra-antennary glycans and glycans containing α1,3 fucose

Not wishing to be restricted further, but in the interests of clarity, preferred combinations of glycosylation markers for use in aforementioned methods can be selected from the group consisting of:—S3 and S4, fucose, GU of 10.65 and tri and tetra-antenary glycans; A3FG1 and FA2; SLe^{x} and fucosylated agalactosylated biantennary glycans or the like; or combinations thereof.

For the avoidance of doubt, reference to “two or more” glycosylation markers means that the methods are concerned with two or more types of glycosylation markers, and not concerned with two or more instances of the same glycosylation marker.

Following further extensive experimentation, the inventors have identified that by monitoring a change in glycosylation that is associated with the development and progression of cancerous or malignant conditions (i.e. glycosylation markers), and a cancer marker that is not defined by a change in glycosylation, one can arrive at sensitive and specific diagnostic methodologies.

Therefore, according to a fourth aspect of the present invention there is provided a method for the diagnosis of a cancerous and/or malignant condition, the method comprising the steps of:—
providing a test sample from a subject,
determining the level in the test sample of one or more glycosylation marker(s) of a cancerous and/or malignant condition and one or more non-glycosylation marker of a cancerous and/or malignant condition, and
providing a diagnosis based on the determined level of the one or more glycosylation markers and the one or more non-glycosylation markers.

The inventors have further identified that, in addition to the diagnosis of a cancerous and/or malignant condition, the methods and markers of the present invention have further utility in relation to methods for the prognosis of a cancerous condition in a subject.

As such, according to a fifth aspect of the present invention there is provided a method for the prognosis of a cancerous or malignant condition in a subject, the method comprising the steps of:
providing a test sample from a subject,
determining the level in the test sample of one or more glycosylation marker(s) of a cancerous and/or malignant conditions and one or more non-glycosylation marker of a cancerous and/or malignant condition, and
determining the prognosis from the determined level of the one or more glycosylation markers and the one or more non-glycosylation markers.

Furthermore, the markers and methods of the present invention have utility in methods for monitoring the response by a subject to the treatment of a cancerous or malignant condition.

As such, according to a sixth aspect of the present invention there is provided a method for determining the response to therapy of a subject whom has been administered a therapeutic compound for the treatment of a cancerous and/or malignant condition, the method comprising the steps of:
providing a test sample from a subject,
determining the level in the test sample of one or more glycosylation marker of a cancerous and/or malignant condition and one or more non-glycosylation marker of a cancerous and/or malignant condition,
determining the response from the determined level of the one or more glycosylation markers and the one or more non-glycosylation markers.

Using a glycosylation marker combined with a non-glycosylation marker has been found to provide an improved (e.g. more specific and/or sensitive) method of diagnosing, of prognosing and/or of determining the response to a therapy, than can be achieve by methods that are concerned with only a glycosylation marker and/or multiple glycosylation markers. The level of improvement may be additive, but is preferably synergistic.

It is preferred that the method of the fourth, fifth and sixth aspects of the present invention determine a level in the test sample of (and so determine the diagnosis, prognosis or response on the basis of) 2, 3, 4, 5, 6, 7, 8, 9, 10 or more non-glycosylation markers for a cancerous and/or malignant condition, and/or glycosylation markers for a cancerous and/or malignant condition, preferably 5 to 10 glycosylation markers and/or 5 to 10 non-glycosylation markers. Methods that involve the determination on the basis of all (i.e. total or unpurified) glycosylation markers in a biological sample are however not preferred. Indeed, it is preferred that the determination is carried out on less than 40, 30, 20 or 15 glycosylation and/or non-glycosylation markers for a cancerous and/or malignant condition.

The glycosylation markers used the methods of the fourth, fifth and sixth aspects of the present invention can be any of those mentioned above with respect to the first, second and third aspects of the present invention, including all preferred or optional embodiments thereof.

Non-glycosylation markers of a cancerous and/or malignant condition are any markers of a cancerous and/or malignant condition that are not characterised as a marker because they represent a change in the glycosylation of a glycoprotein that is associated with the development and/or progression of a cancerous and/or malignant condition. Such non-glycosylation markers will be known to the skilled person and/or described in the present specification. Not wishing to be restricted further, but in the interests of clarity, these markers may be selected from the group consisting of inflammatory markers, cytokines, chemokines, genetic markers, Catecholamines, Immunoglobulins, markers for angiogenesis or the like, or any combination thereof. More specifically, for example, suitable markers may be selected from the group consisting of: Alphafetoprotein, NMP22, Carcinoembryonic antigen (CEA), HER-2, CA 15-3, CA 27-29, CA 125, CA 19-9, and C-reactive protein (CRP), IL-4, IL-10, IL-1α and IL-1β, MCP-1, or the like; or any combination thereof.

Not wishing to be restricted further, but in the interests of clarity, preferred combinations of glycosylation and non-glycosylation markers for use in aforementioned methods can be selected from the group consisting of:—CRP and any one, two, three, four, five, six, or more of any of the aforementioned glycosylation markers; CRP and any of one, two or three of the glycosylation markers S3 and S4, fucose, GU of 10.65, tri and tetraantenary glycans; S3 and S4, fucose, GU of 10.65, tri and tetraantenary glycans, and CRP; fucosylated agalactosylated biantennary glycans and CRP; one or more pro-inflammatory cytokines (such as IL-1α and IL-1β) and one or more gylcosylation marker selected from above; one or more anti-inflammatory cytokine (such as IL-4 and IL-10) and one or more gylcosylation marker selected from above; one or more chemokine (such as MCP-1) and one or more gylcosylation marker selected from above; or the like; or combinations thereof.

For the avoidance of doubt, reference to “one or more” non-glycosylation markers means that the methods are concerned with one or more types of non-glycosylation markers, and not concerned with one or more instances of the same glycosylation marker.

Yet further extensive experimentation by the inventors has lead to the identification of a number of changes in glycosylation and cytokine expression profiles which have been associated with the development and progression of cancer and chronic inflammation. Having identified these changes, the inventors have recognised their utility as markers for use in improved methods of diagnosing cancer and chronic inflammation and, further, of monitoring the response of these diseases to treatment.

Accordingly in a seventh aspect of the present invention there is provided a method for the diagnosis of a cancerous and/or malignant condition, the method comprising the steps of:
providing a test sample from a subject,
determining the level of at least one marker selected from the group comprising glycans with GU values greater than 10.65, SLe^{x} structures, A2FG1 derived from digestion of SLe^{x}, A3FG1 derived from digestion of SLe^{x}, A4FG1 derived from digestion of SLe^{x}, sialylated tri-antennary glycans, sialylated tetra-antennary glycans, glycans containing α1,3 fucose, α1,3 monofucosylated tri-antennary glycans, α1,3 difucosylated tri-antennary glycans, α1,3 monofucosylated tetra-antennary glycans, α1,3 difucosylated tetra-antennary glycans, tetra-antennary glycans with lactosamine extensions, ratio of α2,3 sialylated glycans to α2,6 sialylated glycans, agalactosylated fucosylated biantennary glycans, core fucosylated agalactosylated biantennary glycans, core fucosylated monosialylated glycans on transferrin, SLe^{x} on glycans on haptoglobin β-chain, A3FG1 derived from digestion of SLe^{x} on glycans on haptoglobin β-chain, A4FG1 derived from digestion of SLe^{x} on glycans on haptoglobin β-chain, SLe^{x} on glycans on α1-acid glycoprotein, A3FG1 derived from digestion of SLe^{x} on glycans on α1-acid glycoprotein, SLe^{x} on glycans on α1-antichymotrypsin, A3FG1 derived from digestion of SLe^{x} on glycans on α1-antichymotrypsin, tetra-antennary tetragalactosylated glycans on α1-antitrypsin, core fucosylated agalactosylated biantennary glycans on IgG, agalactosylated glycans on IgG, sialylation on glycans on IgG, galactosylation on glycans on IgG, FA2G2S1 on glycans on transferrin, FA2BG2S1 on glycans on transferrin and A4G4 glycans on α1-antitrypsin, or the like, or any combination thereof, and
providing a diagnosis based on the determined level of the at least one marker.

The inventors have further identified that, in addition to the diagnosis of a cancerous or malignant condition, the methods and markers of the present invention have further utility in relation to methods for the prognosis of a cancerous condition in a subject.

As such, according to an eighth aspect of the present invention there is provided a method for the prognosis of a cancerous or malignant condition in a subject, the method comprising the steps of:
providing a test sample from a subject,
determining a level in the test sample of at least one marker mentioned in the seventh aspect of the present invention, and
determining the prognosis from the level of the at least one marker.

Furthermore, the markers and methods of the present invention have utility in methods for monitoring the response by a subject to the treatment of a cancerous or malignant condition in a subject,

According to a ninth aspect of the present invention there is provided a method for determining the response to therapy of a subject whom has been administered a therapeutic compound for the treatment of a cancerous or malignant condition, the method comprising the steps of:
providing a test sample from a subject,
determining the level in the test sample of at least one marker mentioned in the seventh aspect of the present invention, and
determining the response from the level of the at least one or more markers.

In a preferred embodiment of the seventh, eighth and ninth aspects of the present invention, the methods include the determination based on only one of the glycosylation markers mentioned in these aspects of the present invention.

The glycosylation markers may be associated with O- and/or N-linked glycans.

For the avoidance of doubt, reference to “at least one” and “only one” glycosylation marker means that the methods are concerned with at least one type of glycosylation marker, and not concerned with at least one instance (i.e. single molecular event) of a glycosylation marker.

The inventors have found that individual changes in the glycosylation of glycoproteins associated with cancerous and/or malignant condition can be common to many glycoproteins. Thus, the glycosylation markers according to any of the aspects of the present invention are preferably not restricted to those present on specific glycoproteins. In the interests of clarity, however, the glycosylation markers are preferably those that are associated with glycoproteins selected from the group consisting of:—acute phase glycoproteins (e.g. serum amyloid A, haptoglobin, α1-acid glycoprotein, α1-antitrypsin, α1-antichymotrypsin, fibrinogen, transferrin, 2-macroglobulin, prothrombin, factor VIII, von Willebrand factor or plasminogen), other serum protein(s) associated with a cancerous and/or malignant condition, Alphafetoprotein, NMP22, Carcinoembryonic antigen (CEA), HER-2, CA 15-3, CA 27-29, CA 125, CA 19-9, and C-reactive protein (CRP), IgG, or the like, or any combination thereof.

The present invention extends to the use of the markers and combinations of markers as identified herein by the inventors in methods for the diagnosis and/or prognosis of at least one cancerous or malignant condition.

Accordingly, a tenth aspect of the invention provides for the use, in a method for the diagnosis of a cancerous and/or malignant condition, of (1) two or more of the glycosylation markers according to the first aspect of the present invention, including any preferred or optional embodiments thereof, (2) one or more glycosylation marker and one or more non-glycosylation marker according to the fourth aspect of the present invention, including any preferred or optional embodiments thereof or, (3) at least on glycosylation marker according to the seventh aspect of the present invention, including any preferred or optional embodiments thereof.

Accordingly, a eleventh aspect of the invention provides for the use, for the prognosis of a cancerous or malignant condition, of (1) two or more of the glycosylation markers according to the second aspect of the present invention, including any preferred or optional embodiments thereof, (2) one or more glycosylation marker and one or more non-glycosylation marker according to the fifth aspect of the present invention, including any preferred or optional embodiments thereof or, (3) at least one glycosylation marker according to the eighth aspect of the present invention, including any preferred or optional embodiments thereof.

Accordingly, a twelfth aspect of the invention provides for the use, in a method for determining the response in a subject to a therapeutic compound administered to said subject for the treatment of a cancerous or malignant condition, of (1) two or more of the glycosylation markers according to the third aspect of the present invention, including any preferred or optional embodiments thereof, (2) one or more glycosylation marker and one or more non-glycosylation marker according to the sixth aspect of the present invention, including any preferred or optional embodiments thereof or, (3) at least on glycosylation marker according to the ninth aspect of the present invention, including any preferred or optional embodiments thereof.

In a thirteenth aspect of the present invention there is provided a kit for diagnosing at least one cancerous condition, the kit comprising:—
means for detecting (1) two or more of the glycosylation markers according to the first aspect of the present invention, including any preferred or optional embodiments thereof, (2) one or more glycosylation marker and one or more non-glycosylation marker according to the fourth aspect of the present invention or, including any preferred or optional embodiments thereof, (3) at least on glycosylation marker according to the seventh aspect of the present invention, including any preferred or optional embodiments thereof and,
instructions for the use of the same.

Methods according to any of the aspects of the present invention that involve the analysis of more than one marker (glycosylation or non-glycosylation markers), can involve the separate, simultaneous or sequential analysis of each marker.

In the aspects of the present invention methods are provided that involve the analysis of a level of one, two or more glycosylation marker, optionally in combination with the analysis of a level of one or more non-glycosylation marker, in order to provide a diagnosis, prognosis or determination of a response to a therapeutic composition.

The skilled person would be well aware, particularly in light of the results provided in the present specification, what levels are to be analysed. In the interests of clarity, however, the levels to be analysed may, for example, be:—the amount of a marker in a sample; the ratio of the amount of one marker to the amount of at least one further marker in a sample; the percentage amount of a marker in a pool of markers (which may be from a total glycoprotein pool) or; the number, position and/or height or integration of peaks that represent one or more marker in a chromatography trace. The level in the test sample of the one or more markers can be determined by essentially any convenient technique or combination of techniques. For example, the markers can be detected by performing chromatography (e.g., normal phase or weak anion exchange HPLC), mass spectrometry, gel electrophoresis (e.g., one or two dimensional gel electrophoresis), capillary electrophoresis and/or an immunoassay or ELISA (e.g., immuno-PCR, ELISA, lectin ELISA, Western blot, or lectin immunoassay) on the sample or a derivative or component thereof (e.g., serum, a serum fraction, a cell or tissue lysate, a glycan pool, an isolated protein, etc.). See, e.g., the examples hereinbelow, as well as U.S. patent application publications 20060269974 by Dwek et al. entitled “Glycosylation markers for cancer diagnosing and monitoring”, 20060270048 by Dwek et al. entitled “Automated strategy for identifying physiological glycosylation marker(s),” and 20060269979 by Dwek et al. entitled “High throughput glycan analysis for diagnosing and monitoring rheumatoid arthritis and other autoimmune diseases”.

The skilled person would be well aware, particularly in light of the results provided in the present specification, how the determination of these levels would indicate a diagnosis, prognosis or a response to a therapeutic compound.

For the avoidance of doubt, however, the methods of all aspects of the present invention preferably involve the step of determining a difference (or change) between the level of one, two or more glycosylation markers, optionally in combination with a difference (or change) in the level of one or more non-glycosylation markers, compared to the level of one or more glycosylation markers and/or non-glycosylation marker of one or more control samples. A control sample may be a sample derived from one or more non-diseased subjects, or a sample obtained previously from the subject; e,g, during a period when the subject did not have cancerous or malignant condition, or was at an earlier stage in the condition.

Differences, or changes in the levels can be an increase or a decrease in those levels. Such differences or changes can manifest themselves as a different amount of a marker, a different ratio between the amount of two markers, a different number of spikes in a particular region of a chromatography trace, a different height in a spike in a chromatography trace.

Thus, the methods of diagnosis, prognosis and monitoring of the present invention may include the step of comparing the level of the one or more markers in the test sample and comparing this level with one or more markers in a control sample and determining the diagnosis, prognosis and/or response based on the difference between those levels.

For example, a difference between the level of a marker identified as being associated with disease (e.g. cancer and/or malignancy) in a sample from a subject, and the level of that marker in a control sample taken from a healthy individual can determine a positive diagnosis for that disease in the subject.

For example, a difference between the levels of a marker indentified as being associated with a disease (e.g. cancer and/or malignancy) in a sample from a diseased subject, and a level of that marker in a control sample taken from the subject earlier can calibrate the progression or regression of the disease.

For example, a difference between the levels of a marker identified as being associated with a disease (e.g. cancer and/or malignancy) in a sample from a diseased subject following treatment with a therapeutic compound, and a level of that marker in a control sample taken from the subject prior to administration of the therapeutic compound earlier can calibrate the response by the individual to the therapeutic compound (i.e. determine if the therapeutic compound is treating the disease).

For example, an increase in the level of the one or more markers in the test sample as compared to the control sample indicates the presence of lung cancer, stage 4 lung cancer and/or stage 3 lung cancer.

Any change in the levels of marker may be indicative of a positive diagnosis, progression or regression of disease or response to a therapeutic compound would be understood by the person skilled in the art.

Not wishing to be restricted further, but in the interests of clarity, for example, in one embodiment, the difference or change could be an increase in the level of one or more of glycans with GU values greater than 10.65, SLe^{x} structures, A3FG1 derived from digestion of SLe^{x}, A4FG1 derived from digestion of SLe^{x}, sialylated tri-antennary glycans, sialylated tetra-antennary glycans, glycans containing α1,3 fucose, SLe^{x} on glycans on haptoglobin β-chain, A3FG1 derived from digestion of SLe^{x} on glycans on haptoglobin β-chain, A4FG1 derived from digestion of SLe^{x} on glycans on haptoglobin β-chain, pro-inflammatory cytokines, IL-4 and IL-10 indicates the presence of cancer, e.g., lung cancer and more preferably stage 4 lung cancer and/or stage 3 lung cancer. The glycans with GU values greater than 10.65 and/or the glycans containing α1,3 fucose may comprise haptoglobin glycans.

In another exemplary embodiment, an increase in the level of one or more of α1,3 difucosylated tri-antennary glycans, SLe^{x} structures, A2FG1 derived from digestion of SLe^{x}, A3FG1 derived from digestion of SLe^{x}, α1,3 monofucosylated tri-antennary glycans, α1,3 monofucosylated tetra-antennary glycans, α1,3 difucosylated tetra-antennary glycans, tetra-antennary glycans with lactosamine extensions, ratio of α2,3 sialylated glycans to α2,6 sialylated glycans and agalactosylated fucosylated biantennary glycans indicates the presence of cancer, e.g., breast cancer. Typically for breast cancer, the increase of α2,3 sialylation is most significant in the tri-sialylated fraction.

In yet another exemplary embodiment, an increase in core fucosylated agalactosylated biantennary glycans, a decrease in core fucosylated monosialylated glycans on transferrin, an increase in SLe^{x} on glycans on haptoglobin β-chain, an increase in SLe^{x} structures, an increase in A3FG1 derived from digestion of SLe^{x}, an increase in A3FG1 derived from digestion of SLe^{x} on glycans on haptoglobin β-chain, an increase in SLe^{x} on glycans on α1-acid glycoprotein, an increase in A3FG1 derived from digestion of SLe^{x} on glycans on α1-acid glycoprotein, an increase in SLe^{x} on glycans on α1-antichymotrypsin, an increase in A3FG1 derived from digestion of SLe^{x} on glycans on α1-antichymotrypsin, an increase in tetra-antennary tetragalactosylated glycans on α1-antitrypsin, an increase in core fucosylated agalactosylated biantennary glycans on IgG, an increase in agalactosylated glycans on IgG, a decrease in sialylation on glycans on IgG, a decrease in galactosylation on glycans on IgG, a decrease in FA2G2S1 on glycans on transferrin, a decrease in FA2BG2S1 on glycans on transferrin, a change in the ratio of α2,3 sialylated glycans to α2,6 sialylated glycans and/or an increase in A4G4 on glycans on α1-antitrypsin indicates the presence of cancer, e.g., ovarian cancer.

Typically, for example in ovarian cancer, the change in the ratio of α2,3 sialylated glycans to α2,6 sialylated glycans comprises a decrease in the ratio of α2,3 sialylated glycans to α2,6 sialylated glycans. In ovarian cancer, the decrease of α2,3 sialylation is typically in the di-sialylated fraction.

Further differences can be extrapolated directly from the results provide in the examples and accompanying figures (without necessarily being restricted to the disease state or glycoprotein of the example or figure)

The person skilled in the art would be aware that any statistically significant difference in level from control would be determinant of a diagnosis, prognosis or response. The degree of difference in the level of one or more marker(s) in a sample from a subject from the level of that marker(s) in a control that would be indicative of a significant change or difference and be determinant of a diagnosis, prognosis or response would be well within the skill of the ordinary person to determine. For the avoidance of doubt, and in the interests of clarity, it is preferred that a significant change is one in which the determined level of marker(s) varies by more than 5, 10, 15 or 20% from that of the control marker(s).

In one embodiment of any of the aspects of the present invention that involve analysis based on more than a single glycosylation marker, or a combination of one or more glycosylation markers and one or more non-glycosylation marker, it is preferred that the method further comprises the step of performing cluster analysis to characterise interplay or an interrelationship between at least two of the markers. Most preferably, cluster analysis is used to show the interplay or an interrelationship between the markers in a sample from the subject and those in a sample from the control. Such cluster analyses can therefore be used to identify differences (or changes) between the markers in a subject sample and those in a control sample and so determine the diagnosis, prognosis or response to a therapeutic agent.

In particular embodiments, cluster analysis of cytokine levels may be performed, including, but not limited to, analysis of pro- or anti-inflammatory cytokines, such as, but not limited to, IL-1α and IL-1β, anti-inflammatory cytokines, such as IL-4 and IL-10, and chemokines, such MCP-1. This may be combined with cluster analysis of glycosylation markers.

A form of cluster analysis based on cluster analysis through a series of individual parameters is explained later in the specification (see also, Cluster—Statistical 5.0, Statsoft Inc., USA).

A particularly preferably form of cluster analysis, however, takes the form of Partial Least Square Projections (PLS projections). PLS projections may be performed on data derived from any number of markers derived from the subject and control. Data for the level for each marker may first be attributed a Variable Importance Plot (VIP) before being subjected to PLS. PLS-DA analysis is described in Hoskuldsson, A. “PLS regression methods.”, J. Chemometr., 2 (1988) 211-228, and in Wold, S., Sjöstrom, M., and Eriksson, L., “PLS regression: A basic tool of chemometrics, Chemometrics and Intelligent Laboratory Systems”, 58, 109-130, 2001.

The diagnosis, prognosis, or response can include, but is not limited to, a determination of, for example, the type of cancer, clinical status (cancer, precancerous condition, benign condition, no condition) or stage of cancer.

The inventors have identified that markers may not be restricted to specific cancers. The cancer, cancerous condition or malignant condition can be, but is not limited to, lung cancer, breast cancer, ovarian cancer, pancreatic cancer, prostate cancer, uveal melanoma, hepatocellular carcinoma, bladder cancer, renal cancer, colon cancer or stomach cancer, neuroblastoma, multiple myloma, colorectal cancer, carcinoma, or the like, or any combination thereof.

Analysis of combinations of changes in serum glycome may improve the separation of cancerous and benign tumours. Accordingly, in one embodiment, levels of two or more markers are analysed to determine whether a tumour is benign or malignant. For example, the levels of SLe^{x} and core fucosylated agalactosylated biantennary glycans may be analysed to determine the presence or absence of ovarian cancer. The level of each marker may be analysed simultaneously or sequentially, and optionally using the aforementioned cluster analysis.

The inventors have surprisingly shown that levels of fucosylated agalactosylated biantennary glycans and C reactive protein (CRP) do not correlate in ovarian cancer. Accordingly, in a further or alternative embodiment of the present invention, levels of fucosylated agalactosylated biantennary glycans and CRP may be determined to distinguish ovarian cancer from non-cancerous inflammatory conditions.

Methods according to the present invention that are concerned with determining the response to a therapeutic compound may be practiced on a subject that has previously been diagnosed with cancer, the method comprising:
treating the subject for the cancer;
providing a first test sample from the subject prior to initiation of the treatment and a second test sample from the subject after initiation of the treatment; and
   comparing the level of the one or more markers in the first test sample with that in the second test sample to monitor the subject's response to the treatment (e.g. administration of the therapeutic compound).

The subject in any aspect of the present invention is preferably a mammal, typically a human. The subject may be an animal or cell line, preferably an animal or cell line prepared for use as a model for disease (e.g. a cancer cell line, or transformed organism) or used in any bioprocessing (e.g. a transformed organism).

The methods of the present invention may be used to monitor consistency of bioprocessing in the subject, by using any change in level of glycosylation markers as an indication of change in bioprocessing (e.g. a change in the biochemistry of the organism that may affect its ability to undergo the bioprocess required)

The methods of the present invention may be used to monitor the consistency of immortalised cancer cell lines or animal models to grow diseased cells (e.g. cancer cells). Changes in levels of glycosylation markers over time (i.e. measured agains control samples taken for the animal or cell line at an earlier time period) can indicate a change in ability to spontaneously produce cancer cells.

In certain embodiments, the step of providing the sample from the subject can further include the step of obtaining the sample from the patient. The test sample can be obtained by essentially any convenient technique as known in the art, and can include the obtaining of a sample derived from a tissue or bodily fluid or from any other suitable sample which may contain, or which may be reasonably expected to contain glycoproteins.

In certain embodiments, said samples may include, but are not limited to; whole serum, blood plasma, blood, urine, sputum, seminal fluid, seminal plasma, pleural fluid, ascites, nipple aspirate, faeces and saliva.

The particular, type of a body fluid or a tissue can depend on the type of cancer (e.g., lung tissue, breast tissue, ovarian tissue or pancreatic tissue for diagnosis or prognosis of the corresponding cancer). In some embodiments, a sample can be obtained from tumour cells.

In one embodiment, the presence of strong linkage between the pro-inflammatory cytokines, between IL-4 and IL-10 and/or between the pro-inflammatory cytokines and one or more of IL-4, IL-10 and MCP-1 indicates the presence of cancer, e.g., lung cancer and/or stage 4 lung cancer. The term “strong linkage” is used herein to describe a linkage or correlation which is greater than that observed in the absence of cancer and/or chronic inflammation. The levels for each of these cytokines, individual, or any combination thereof, may be determined as part of the methods of the present invention.

The markers can be detected, for example, from the whole sample, from a pool of glycoproteins from the sample or on one or more proteins purified from the sample.

Thus, in one embodiment of any of the aspects of the present invention, a pool of N-linked and/or O-linked glycans is released from total glycoproteins in the test sample (e.g., from serum without purifying the glycoproteins by digestion with a glycosidase) and the level of the one or more markers in the pool of glycans is determined. As noted, the glycan markers are optionally detected on particular proteins, for example, on one or more acute phase proteins (e.g., serum amyloid A, haptoglobin, α1-acid glycoprotein, α1-antitrypsin, α1-antichymotrypsin, fibrinogen, transferrin, 2-macroglobulin, prothrombin, factor VIII, von Willebrand factor or plasminogen) or other serum protein(s) of interest).

The markers on particular proteins can be detected with or without purification of the proteins from the sample. Thus, in one embodiment, one or more proteins (e.g., one or more acute phase proteins) are isolated from the test sample prior to determining the level of the one or more markers on the proteins. Affinity purification of acute phase proteins to isolate them prior to high-throughput analysis of glycan markers by HPLC is described in the examples herein. The sample can be treated as necessary prior to detection of the markers, for example, cells and/or tissues are optionally lysed for detection of intracellular glycoprotein markers.

The level in the test sample of the one or more markers can be determined by essentially any convenient technique or combination of techniques. For example, the markers can be detected by performing chromatography (e.g., normal phase or weak anion exchange HPLC), mass spectrometry, gel electrophoresis (e.g., one or two dimensional gel electrophoresis), capillary electrophoresis and/or an immunoassay (e.g., immuno-PCR, ELISA, lectin ELISA, Western blot, or lectin immunoassay) on the sample or a derivative or component thereof (e.g., serum, a serum fraction, a cell or tissue lysate, a glycan pool, an isolated protein, etc.). See, e.g., the examples hereinbelow, as well as U.S. patent application publications 20060269974 by Dwek et al. entitled “Glycosylation markers for cancer diagnosing and monitoring”, 20060270048 by Dwek et al. entitled “Automated strategy for identifying physiological glycosylation marker(s),” and 20060269979 by Dwek et al. entitled “High throughput glycan analysis for diagnosing and monitoring rheumatoid arthritis and other autoimmune diseases”.

Databases that store “fingerprints” for specific glycosylation markers may be used to analyse the presence of markers from the methods discussed above. For example, chromatography, mass spectrometry, gel electrophoresis, capillary electrophoresis and/or an immunoassay results for a number of specific known glycosylation markers may be retained on a database. The methods of the present invention may include the step of interrogating such a database in order to match the chromatography, mass spectrometry, gel electrophoresis, capillary electrophoresis and/or immunoassay results derived from the subject and/or control sample with those in the database, and thereby identify which markers are present.

The methods are optionally used to monitor response of a subject to treatment. Thus, in one class of embodiments wherein the subject has previously been diagnosed with cancer, the methods include treating the subject for the cancer, obtaining a first test sample from the subject prior to initiation of the treatment and a second test sample from the subject after initiation of the treatment and comparing the level of the one or more markers in the first test sample with that in the second test sample to monitor the subject's response to the treatment.

Optionally, the level in the test sample of two or more (e.g., three, four, five, or six or more) of the markers described herein is determined.

Similarly, the markers described herein can be used in combination with other markers for the cancer, e.g., glycosylation, genetic and/or protein markers. Thus, for example, the methods can include determining a level in the test sample, or in another clinical sample from the subject, of one or more additional markers and determining the diagnosis, prognosis and/or response from the level of the one or more markers and the level of the one or more additional markers. Useful additional markers include, for example, CA 15-3, CEA, and/or C reactive protein for breast cancer) and CA125 and/or C reactive protein for ovarian cancer and C reactive protein for lung cancer.

In a fourteenth aspect of the invention, there is provided methods of assessing the inflammatory state of a subject using at least one of the markers of the invention. Thus, in certain further embodiments there is provided methods that include providing a test sample from the subject; determining a level in the test sample of one or more markers selected from the group consisting of: glycans with GU values greater than 10.65, SLe^{x} structures, A2FG1 derived from digestion of SLe^{x}, A3FG1 derived from digestion of SLe^{x}, A4FG1 derived from digestion of SLe^{x}, sialylated tri-antennary N glycans, sialylated tetra-antennary glycans, glycans containing α1,3 fucose, α1,3 monofucosylated tri-antennary glycans, α1,3 difucosylated tri-antennary glycans, α1,3 monofucosylated tetra-antennary glycans, α1,3 difucosylated tetra-antennary glycans, tetra-antennary glycans with lactosamine extensions, ratio of α2,3 sialylated glycans to α2,6 sialylated glycans, agalactosylated fucosylated biantennary glycans, core fucosylated agalactosylated biantennary glycans, core fucosylated monosialylated glycans on transferrin, SLe^{x} on glycans on haptoglobin β-chain, A3FG1 derived from digestion of SLe^{x} on glycans on haptoglobin β-chain, A4FG1 derived from digestion of SLe^{x} on glycans on haptoglobin β-chain, SLe^{x} on glycans on α1-acid glycoprotein, A3FG1 derived from digestion of SLe^{x} on glycans on α1-acid glycoprotein, SLe^{x} on glycans on α1-antichymotrypsin, A3FG1 derived from digestion of SLe^{x} on glycans on α1-antichymotrypsin, tetra-antennary tetragalactosylated glycans on α1-antitrypsin, core fucosylated agalactosylated biantennary glycans on IgG, agalactosylated glycans on IgG, sialylation on glycans on IgG, galactosylation on glycans on IgG, FA2G2S1 on glycans on transferrin, FA2BG2S1 on glycans on transferrin and A4G4 on glycans on α1-antitrypsin; and comparing the level of the one or more markers in the test sample with a level of the one or more markers in a control sample, wherein an increase in the level of the one or more markers in the test sample as compared to the control sample is indicative of chronic inflammation.

The method may further comprise diagnosing, prognosing and/or monitoring response to treatment of a cancer or chronic inflammatory disease in the subject based on the level of the one or more markers (e.g., a cancer as noted above, rheumatoid arthritis, inflammatory gynaecologic benign diseases such as endometriosis or cysts, Chronic Obstructive Pulmonary Disease, Osteoarthritis, Inflammatory Bowel Disease (Ulcerative Colitis and Chron's Disease), Psoriasis, Tuberculosis, Chronic Cholecystitis, Bronchiectasis, Silicosis or chronic inflammation caused by a foreign body implanted in a wound).

Essentially all of the embodiments noted for the first to thirteenth aspects of the present invention, are as for the fourteenth aspect mutatis mutandis. When the context for such embodiments appear inconsistent with the fourteenth aspect because of reference to a cancerous and/or malignant condition (or related terms) this may be replaced with reference to a chronic inflammatory condition (or related term). For example with respect to number of markers detected, use in combination with other markers of chronic inflammation (e.g., C reactive protein), type of sample, technique(s) employed to detect the markers and/or the like.

Compositions are another feature of the invention, e.g., compositions useful in practicing or formed while practicing the methods of any aspect of the present the invention. For example, a composition of the invention optionally includes an antibody against one of the markers of the invention, or more that one antibody each one raised against separate markers of the methods, optionally in combination with other reagents for determining the level of the marker in a sample.

Thus, one exemplary general class of embodiments provide a composition that comprise a first antibody against a first glycoform of a first protein, which glycoform comprises one or more of: glycans with GU values greater than 10.65, SLe^{x} structures, A2FG1 derived from digestion of SLe^{x}, A3FG1 derived from digestion of SLe^{x}, A4FG1 derived from digestion of SLe^{x}, sialylated tri-antennary glycans, sialylated tetra-antennary glycans, glycans containing α1,3 fucose, α1,3 monofucosylated tri-antennary glycans, α1,3 difucosylated tri-antennary glycans, α1,3 monofucosylated tetra-antennary glycans, α1,3 difucosylated tetra-antennary glycans, tetra-antennary glycans with lactosamine extensions, ratio of α2,3 sialylated glycans to α2,6 sialylated glycans, agalactosylated fucosylated biantennary glycans, core fucosylated agalactosylated biantennary glycans, core fucosylated monosialylated glycans on transferrin, SLe^{x} on glycans on haptoglobin β-chain, A3FG1 derived from digestion of SLe^{x} on glycans on haptoglobin β-chain, A4FG1 derived from digestion of SLe^{x} on glycans on haptoglobin β-chain, SLe^{x} on glycans on α1-acid glycoprotein, A3FG1 derived from digestion of SLe^{x} on glycans on α1-acid glycoprotein, SLe^{x} on glycans on α1-antichymotrypsin, A3FG1 derived from digestion of SLe^{x} on glycans on α1-antichymotrypsin, tetra-antennary tetragalactosylated glycans on α1-antitrypsin, core fucosylated agalactosylated biantennary glycans on IgG, agalactosylated glycans on IgG, sialylation on glycans on IgG, galactosylation on glycans on IgG, FA2G2S1 on glycans on transferrin, FA2BG2S1 on glycans on transferrin and A4G4 on glycans on α1-antitrypsin.

In one embodiment, lectins can be used to distinguish between α2,3 sialylated N-linked glycans and α2,6 sialylated N-linked glycans. For example, Maackia Amurensis Lectin II may be used in the case of α2,3 sialylated N-linked glycans and Sambucus Nigra bark lectin may be used in the case of α2,6 sialylated N-linked glycans. α2,3 and α2,6 sialylation relate to whole serum.

The composition optionally includes the first glycoform of the first protein (e.g., an acute phase protein or other serum protein or protein of interest), a sample from a subject, a lectin, a secondary antibody against the first antibody, a nucleic acid tag associated with the first antibody (covalently or noncovalently, and optionally distinguishable from any other tags on other antibodies in the composition for multiplex assays), a second antibody against a second glycoform of the first protein and/or a third antibody against a glycoform of a second protein. A secondary antibody or lectin is optionally labelled, e.g., with a fluorescent label or enzyme or is configured to bind a label (e.g., is biotinylated). The composition can include reagents for amplifying a nucleic acid tag or tags (e.g., a polymerase, nucleotides, etc.), reagents for detecting a lectin or secondary antibody (e.g., a fluorogenic or colorimetric substrate) or the like.

Kits comprising one or more elements of the compositions are also features of the invention. For example, a kit can include an antibody as described above, and optionally also a lectin, a secondary antibody against the first antibody, a second antibody against a second glycoform of the first protein, a third antibody against a glycoform of a second protein, reagents for amplifying a nucleic acid tag or tags, reagents for detecting a lectin or secondary antibody and/or the like, packaged in one or more containers. Typically, the kit includes instructions for using the components of the kit to diagnose, prognose, or monitor a cancer or inflammatory condition.

Systems for performing the above correlations are also a feature of the invention. Typically, the system will include system instructions that correlate the levels of one or more markers of the invention with a particular diagnosis, prognosis, etc. The system instructions can compare detected information as to marker levels with a database that includes correlations between the markers and the relevant phenotypes. The system includes provisions for inputting sample-specific information regarding marker detection information, e.g., through an automated or user interface, and for comparing that information to the database.

The system can include one or more data acquisition modules for detecting one or more marker levels. These can include sample handlers (e.g., fluid handlers), robotics, microfluidic systems, protein purification modules, detectors, chromatography apparatus, mass spectrometers, thermocyclers or combinations thereof, e.g., for acquiring samples, diluting or aliquoting samples, purifying marker materials (e.g., proteins), detecting markers and the like. The sample to be analyzed, or a composition as noted above, is optionally part of the system, or can be considered separate from it.

Optionally, system components for interfacing with a user are provided. For example, the systems can include a user viewable display for viewing an output of computer-implemented system instructions, user input devices (e.g., keyboards or pointing devices such as a mouse) for inputting user commands and activating the system, etc. Typically, the system of interest includes a computer, wherein the various computer-implemented system instructions are embodied in computer software, e.g., stored on computer readable media.

Any of the aforementioned glycans described in the apectes of the present invention above may be N- or O-linked.

### ABBREVIATIONS

2-AB-2-Amino Benzamide, ABS— Arthrobacter ureafaciens Sialidase, AMF— Almond Meal α-Fucosidase, BKF— Bovine Kidney Fucosidase, BTG -β-Galactosidase, CHAPS-3-[(3-cholamidopropyl)dimethylammonio]-1-propanesulfonate, CRP—C Reactive Protein, DTT-Dithiothreitol, EDTA-ethylenediaminetetraacetic acid, EGF—Epidermal Growth Factor, FUT3-α(1,3) Fucosyltransferase, GlcNAc—N-Acetyl Glucosamine, GU—Glucose Unit, GUH-β-N-acetylglucosaminidase cloned from Streptococcus pneumonia, expressed in E. coli, HIV—Human Immunodeficiency Virus, HPLC—High Performance Liquid Chromatography, IEF—isoelectric focusing, IgG—Immunoglobulin G, IL—Interleukin, IFN-γ—Interferon-γ, IPG—immobilized pH gradient, JBM—Jack Bean α-Mannosidase, MALDI—matrix-assisted laser desorption-ionization, MCP-1—Monocyte Chemoattractant Protien-1, MIP-1—Macrophage Inflammatory Protein-1, MS—mass spectrometry, NAN1—Streptococcus pneumoniae sialidase, NP-HPLC—Normal Phase HPLC, NSAID—non steroidal anti-inflammatory drugs, PAGE—polyacrylamide gel electrophoresis, RT—Room Temperature, SDS-PAGE—Sodium Dodecyl Sulphate PAGE, sILR—Soluble IL6 Receptor, SLe^{x}(SLex)—Sialyl Lewis X, SPG—Streptococcus pneumoniae β-galactosidase, ST3GaIIV—α(2, 3)-sialyltransferase IV, TOF—time-of-flight, TNF-Tumor Necrosis Factor, WAX-Weak Anion Exchange Chromatography, XMF—Xanthomonus sp. alpha-fucosidase.

Structure abbreviations: all N-glycans have two core GlcNAcs; F at the start of the abbreviation indicates a core fucose α1-6 linked to the inner GlcNAc; Mx, number (x) of mannose on core GlcNAcs; Ax, number of antenna (GlcNAc) on trimannosyl core; A2, biantennary with both GlcNAcs as β1-2 linked; A3, triantennary with a GlcNAc linked β1-2 to both mannose and the third GlcNAc linked β1-4 to the α1-3 linked mannose; B, bisecting GlcNAc linked β1-4 to core mannose; Gx, number (x) of β1-4 linked galactose on antenna; F(x), number (x) of fucose linked α1-3 to antenna GlcNAc; Lac(x), number (x) of lactosamine (Galβ1-4GlcNAc) extensions; Sx, number (x) of sialic acids linked to galactose.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells and the like.

An “amino acid sequence” is a polymer of amino acid residues (e.g., a protein) or a character string representing an amino acid polymer, depending on context.

A “polypeptide” or “protein” is a polymer comprising two or more amino acid residues. The polymer can additionally comprise non-amino acid elements such as labels, quenchers, blocking groups or the like and can optionally comprise modifications such as glycosylation or the like. The amino acid residues of the polypeptide can be natural or non-natural and can be unsubstituted, unmodified, substituted or modified.

The term “glycoprotein” refers to an amino acid sequence and one or more oligosaccharide (glycan) structures associated with the amino acid sequence. A given glycoprotein can have one or more “glycoforms”. Each of the glycoforms of the particular glycoprotein has the same amino acid sequence; however, the glycan(s) associated with distinct glycoforms differ by at least one monosaccharide or linkage.

The term “glycan” refers to a polysaccharide (a polymer comprising two or more monosaccharide residues). “Glycan” can also be used to refer to the carbohydrate portion of a glycoconjugate, such as a glycoprotein or glycolipid. Glycans can be homo- or heteropolymers of monosaccharide residues, and can be linear or branched. “N-linked” glycans are found attached to the R-group nitrogen of asparagine residues in proteins, while “O-linked” glycans are found attached to the R-group oxygen of serine or threonine residues.

The “GU value” (or “glucose unit value”) of a glycan indicates its approximate size. The GU value expresses essentially the elution time of a particular glycan from a chromatography column. Since the elution time expressed in real time or volume can vary depending on the individual column, its age, etc., the column is first calibrated with a standard mixture of glycose oligomers.

A tetra-antennary N-linked glycan with one or more lactosamine extensions is a tetra antennary structure with four galactose and one or more additional Gal-GlcNAc (lactosamine) extensions linked to any one of the four galactose. It can carry up to four sialic acids (A4G(4)₄LacS4).

The term “A2FG1” throughout the specification includes both naturally occurring A2FG1 and A2FG1 obtained by digesting glycans with sialidase, galactosidase and/or α1,2 fucosidase. Accordingly, the term “A2FG1 derived from digestion of SLe^{x}” is understood herein to include A2FG1 naturally present as well as A2FG1 derived from digestion of SLe^{x}.

The term “A3FG1” throughout the specification includes both naturally occurring A3FG1 and A3FG1 obtained by digesting glycans with sialidase, galactosidase and/or α1,2 fucosidase. Accordingly, the term “A3FG1 derived from digestion of SLe^{x}” is understood herein to include A3FG1 naturally present as well as A3FG1 derived from digestion of SLe^{x}.

The term “A4FG1” throughout the specification includes both naturally occurring A4FG1 and A4FG1 obtained by digesting glycans with sialidase, galactosidase and/or α1,2 fucosidase. Accordingly, the term “A4FG1 derived from digestion of SLe^{x}” is understood herein to include A4FG1 naturally present as well as A4FG1 derived from digestion of SLe^{x}.

“Acute-phase proteins” are proteins whose plasma concentrations increase (positive acute phase proteins) or decrease (negative acute phase proteins) in response to inflammation, e.g., by 25% or more.

The term “subject” refers to an animal, more preferably a mammal, and most preferably a human. Typically, the subject is known to have or suspected of having a disease, disorder, or condition of interest, e.g., a cancer or chronic inflammation.

The term “marker” refers to a molecule that is detectable in a biological sample obtained from a subject and that is indicative of a disease, disorder, or condition of interest (or a susceptibility to the disease, disorder, or condition) in the subject. Markers of particular interest in the invention include glycans and glycoproteins showing differences in glycosylation between a sample from an individual with the disease, disorder, or condition and a healthy control.

A “control sample” can originate from a single individual not affected by a disease, disorder, or condition of interest (e.g., cancer or chronic inflammation) or be a sample pooled from more than one such individual.

In the context of the invention, the term “isolated” refers to a biological material, such as a protein, which is substantially free from components that normally accompany or interact with it in its naturally occurring environment. The isolated material optionally comprises material not found with the material in its natural environment, e.g., a cell. A protein isolated from a cell or from serum, for example, can be purified or partially purified from the cell or serum.

As used herein, an “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1999), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein, includes antibodies or fragments either produced by the modification of whole antibodies or synthesized de novo using recombinant DNA methodologies. Antibodies include, e.g., polyclonal and monoclonal antibodies, and multiple or single chain antibodies, including single chain Fv (sFv or scFv) antibodies in which a variable heavy and a variable light chain are joined together (directly or through a peptide linker) to form a continuous polypeptide, as well as humanized or chimeric antibodies.

An “immunoassay” makes use of the specific binding of an antibody to its antigen to identify and/or quantify the antigen in a sample. An immunoassay can involve a single antibody or two or more antibodies (to a single antigen or a plurality of antigens).

A variety of additional terms are defined or otherwise characterized herein.

### BRIEF DESCRIPTION OF THE DRAWINGS

FIG. 1 shows the NP-HPLC exoglycosidase digestion profile of the serum glycome. a) Describes the monosaccharide residue symbols and bond angles of the pictured glycans. 2AB labelled N-linked glycans were digested by exoglycosidases and analysed by NP—HPLC (b) and also weak anion exchange chromatography (c). All structures in each peak have been fully characterised previously by Royle et al. (Royle L. et al.). Pictured are the most significant glycans. S-Number of sialic acids attached to the glycans within the peak. The exoglycosidases used were: ABS—Arthobacter Ureafaciens sialidase (removes sialic acid), BTG—bovine testis β-galactosidase (removes galactose unless there is an α1,3 linked fucose attached), BKF—bovine kidney fucosidase (removes core fucose), AMF—almond meal fucosidase (removes α1,3/4 linked fucose).

FIG. 2 shows a dot plot of characterised glycosylation changes of the control and lung cancer serum and isolated haptoglobin. Plotted are the percentage area for glycan structures as a) part of the total serum N-linked glycan pool (serum glycome) for each individual serum sample from stage 3 (n=15) and stage 4 (n=12) lung cancer patients and healthy control (n=10); and b) Percentage area for glycan structures as part of the total N-linked glycan pool from isolated haptoglobin for control (n=4) and stage 4 lung cancer (n=4). The % of glycans with GU>10.65 were calculated through calculating total area under all peaks over GU>10.65 (FIG. 1a). The % of α1,3 fucose was calculated from the sialidase and β-galactosidase digested serum glycome. The peaks containing α1,3 fucose were identified through α1,3 specific fucosidase digestion (FIG. 1b). The % of tri- and tetra-antennary structures were calculated from the sialidase and β-galactosidase fucosidase digested serum glycome (FIG. 1b). The % of Tri (S3) and Tetra (S4)-sialylated structures were calculated using WAX-HPLC (FIG. 1c). Bars represent the mean of the data set.

FIG. 3 shows SDS PAGE analysis of isolated haptoglobin. Anti-haptoglobin resin (10 μl) incubated with lung cancer and control serum was run on 4-15% Bis-Tris gels (Invitrogen) alongside 7 μl of MultiMark protein ladder. The eluted material from the resin shows the haptoglobin beta and alpha chains. The haptoglobin beta chain was excised and the N-linked glycans were analysed.

FIG. 4 shows NP-HPLC of the serum N-linked glycome and isolated haptoglobin. 2AB labelled N-linked glycans were run on NP-HPLC. The highlighted area represents the glycan structures with GU values >10.65. Depicted are the glycan structures identified in the serum glycome with GU values >10.65 in a healthy volunteer. The figure also shows an example N-linked glycan profile of the haptoglobin β-chain from a healthy volunteer. For glycan nomenclature, see FIG. 1.

FIG. 5 shows serum cytokine levels for control and cancer groups. The serum cytokine concentrations were determined as a supplied service by Endogen® SearchLight™ (Pierce Biotechnology, www.endogen.com). The levels plotted are the mean of five control serum (grey) and five stage 4 lung and breast cancer serum samples (black)±standard error. All values were normalised taking the maximum value of the data set as 100. The chemokines MCP-1, MIP-1α and Rantes are also known as CCL2, CCL3 and CCL5.

FIG. 6 shows cluster analysis of cytokine, chemokine, CRP and sILβR levels in patients and controls. The joining tree clustering was carried out for a) the controls serum, and b) the patients (lung and breast cancer). The clusters of parameters are separated by levels of linkage (by method of average links of suspended grouping). Such clustering reflects the relatedness of certain parameters inside the whole spectrum of chemokines and cytokines involved in the study. Marked are the pro-inflammatory cytokines (bold black line), T_{H}2 cytokines (thin black line) and chemokines (dashed line). The chemokines MCP-1, MIP-1α and Rantes are also known as CCL2, CCL3 and CCL5.

FIG. 7 shows linear regression analysis of CRP with percentage of glycan structures >GU10.65. Cytokine data was correlated with glycosylation data. Significant correlations (P<0.05) were identified for percentage of N-linked glycan structures with GU values >10.65 and serum CRP a) when analysing cancer patients (lung and breast cancer) and healthy controls combined, and b) lung and breast cancer patients alone. On each graph is shown values for the correlation coefficient, probability and equation of each line,

FIG. 8 shows a diagrammatic representation of the interplay of cytokines which modulate the expression of the acute phase proteins and glycosylation machinery. The data in this figure was compiled from data highlighted here and that of (van Dijk W. et al. 1995; Loyer P. et al. 1993; Ishibashis Y. et al. 2005; Abbott et al. 1991 and Wigmore et al. 1997). It should be noted that the effects of each cytokine can be specific and not a general response, in some cases, specifically regulating a precise alteration of a set of acute phase proteins, or glycosylation enzymes. Pictured here are the cumulative effects of the cytokines.

FIG. 9 shows NP-HPLC profiles of N-glycans released from total serum protein from a healthy control and an advanced Breast Cancer patient. The N-glycan pool consists of more than 117 structures, all of which were identified combining NP HPLC with exoglycosidase digestions, WAX HPLC and Mass Spectrometry as described in Royle L. 2006. Glucose units (GU) were obtained by comparing the glycan profiles to a standard Dextran ladder. (a) Upper panel: The undigested N-glycan pool showing the increase in the structure A3F1G3S3 at GU 10.75 in breast cancer. (b) Lower panel: NP profiles of the tri-sialylated fractions of the N-glycan pool from control and patient serum separated by WAX HPLC amplifying the increase in A3F1G3S1 in patient compared to control,

FIG. 10 shows the identification and quantification of the glycan marker, A3FG1. (a) Total N-glycan profiles following sialidase and β-galactosidase digestions for quantification of A3FG1 at GU 7.5, the digested product of A3F1G3S3. A3—triantennary referring to three GlcNAc linked to the trimannosyl core; Gx-number (x) of β1-4 linked galactose on antenna; S(x)-number (x) of sialic acid linked to galactose; and F(x)-number (x) of fucose linked α1-3 to antenna GlcNAc. (b) The A3FG1 peak was collected and further digested sequentially with AMF, JBH and SPG to determine its specific linkages also aided by comparison with known IgG glyans (data not shown). ABS— Arthrobacter ureafaciens sialidase (removes all sialic acid), BTG—Bovine testis beta galactosidase (removes all galactose), AMF—Almond meal fucosidase (removes only α1-3/4 linked fucose), JBH—Jack bean hexosaminidase (removes GlcNAc), and SPG—Streptococcus pneumoniae β-galactosidase (removes only β1-4 linked galactose),

FIG. 11 shows a scatter plot of the glycan marker, A3FG1 quantified from the N-glycan pools of healthy controls (n=19) and Advanced Breast Cancer patients (n=18). Black bars indicate the average levels for each group: control (2.96%±1.65) and advanced breast cancer (6.55%±3.02).

FIG. 12 shows a longitudinal study correlating the glycan marker

A3FG1 (FIG. 12A) and CA 15-3 (FIG. 12B) from whole serum of breast cancer patients (n=10). Two samples were obtained from each patient for evaluation, comprising of an early stage sample following diagnosis and an advanced stage sample after detection of metastasis. (a) A3FG1 levels quantified by exoglycosidae digestion and NP HPLC plotted against duration of disease progression. (b) CA 15-3 measured by the clinical chemistry automated assay (Bayer Centaur) plotted also against the duration of breast cancer.

FIG. 13A shows the results of glycoproteomics to identify candidate proteins carrying the serum glycan marker. 2D PAGE of breast cancer serum (80 μg) stained with the fluorescent dye, OGT 1238 (Oxford Glycosciences, Abingdon, UK). Duplicate gels of breast cancer and control serum were immunoblotted against SLe^{x} using the KM93 antibody, highlighting target proteins (i-iii),

FIG. 13B shows quantifed levels of A3FG1 from the N-glycan pools of α1 acid glycoprotein, α1 anti chymotrypsin and haptoglobin of samples from Patient A excised from a 2D gel, plotted against A3FG1 measured from whole serum and CA 15-3. Patient timeline: (i) Following radiotherapy, mastectomy stage 3 grade 2 (T3P3M1)-invasive breast cancer (at least 100 mm) with tumour in 16 of 20 lymph nodes; (ii) Six months of adjuvant chemotherapy followed by Tamoxifen and a further combination of Zoladex and Tamoxifen; (iii) pain and rising tumour markers with no evidence of progression in bone scans, ultrasound and X-rays; and (iv) metastases detected in liver and the right pleura,

FIG. 14 shows typical NPHPLC chromatograms of glycans previously separated by charge on WAXHPLC from A) control sample and B) stage III ovarian cancer patient samples. (a) whole unfractionated serum glycans, (b) neutral fraction, (c) monosialylated fraction, (d) disialylated fraction, (e) trisialylated fraction. See table 4 for peak ID. All structures in each peak have been fully characterized previously by Royle et al. (Royle et al.). The peaks numbered above correspond to these which were significantly different from controls in all three patients,

FIG. 15 shows a comparison of SLe^{x} (A3FG1), FA2, A3F1G1 together with FA2 and CA125 levels in serum samples (healthy controls, benign gynaecological conditions, borderline ovarian tumours, ovarian cancer (ov ca), primary peritoneal carcinomatosis (PPC), endometrial cancer metastasized to ovary (met to ov) and other gynaecological cancers),

FIG. 16 shows typical NPHPLC chromatograms of serum glycans after sialidase and β1-4 galactosidase digestion from (a) control sample, (b) stage III ovarian cancer patient, (c) malignant melanoma and (d) other gynaecological cancer,

FIG. 17 shows NPHPLC chromatograms of serum glycans released from IgG heavy chain purified by SDS-PAGE. a) control sample and b)-d) stage III ovarian cancer patient samples: G0—agalactosylated, G1—monogalactosylated, G2—digalactosylated and S—sialylated glycan structures,

FIG. 18 shows serum proteins from patient B (stage III ovarian cancer) were separated by 2-DE using 7 cm pH 3-10 nonlinear immobilized pH gradients (pH 3-10 NL IPG) and 4-12% SDS-PAGE gradient gels (multimark marker was used). Gels were stained using a fluorescent dye (OGT 1238) and the images were captured using Fuji LAS-1000 Camera,

FIG. 19 shows NPHPLC chromatograms of serum glycans released from haptoglobin β-chain 2D gel spots from A control and B patient B (stage III ovarian cancer), and

FIG. 20 shows NPHPLC chromatograms of serum glycans released from A) α1-acid glycoprotein 2D gel spots from pooled (a) control, (b) benign, (c) malignant and (d) metastatic samples and from B) α1-antichymotrypsin from pooled malignant samples excised from 2D gel digested by exoglycosidases for structural assignment of the outer arm fucosylated structures. The exoglycosidases used were ABS-removes sialic acid, SPG-removes β1,4 linked galactose, XMF-removes α1,2 linked fucose and AMF-removes α1,3 and α1,4 linked fucose.

FIG. 21 Shows Partial Least Squares—Disciminant Analysis (PLS DA) showing separation between healthy control and lung cancer samples based on a combination of markers selected from the HPLC analysis and CRP using High Sensitivity CRP enzyme immunoassay kit from Biocheck Inc Cat number BC-1119). The relative contribution factors of the markers to the PLS DA plot are shown in lower chart and were determined by a Variable Importance Plot (VIP) analysis. The results demonstrate that multiple markers combining specific sugars from the serum glycome and a non-glycosylation protein marker (CRP) distinguish between lung cancer patients and healthy controls better than CRP or glycans alone.

FIG. 22 WAX fractionation of whole serum glycans, where (a) shows Weak anion exchange (WAX) chromatography separating trace in which total serum glycans are separated into neutral, mono- di and tri sialylated fractions from a sample from a healthy control subject (left hand side) and a subject with advanced ovarian cancer (right hand side). (b)-(e) shows NP HPLC profiles for total serum glycans that are separated into neutral, mono- di and tri sialylated fractions from a sample from a healthy control subject (left hand side) and a subject with advanced ovarian cancer (right hand side). Differences in all glycosylation profiles between control and diseased subject in all fractions are evident. The tri-antennary fractions are circled as an example.

FIG. 23 shows the sensitive but not specific nature of the glycosylation marker triaantennary fucosylated glycan biomarker, results taken from whole serum by WAX HPLC. The comparison of triasialylated fractions from a range of cancers and a healthy control is shown. In all cancer samples the ratio of the triaantennary glycan with the SLex epitope: the triantennary glycan without the SLex epitope is greater than the same ratio found in samples from the healthy control. This marker is therefore common to all cancers investigated.

FIG. 24 Glycan analysis of PSA subforms (F1-F5) from healthy seminal fluid and prostate cancer patient serum. The glycans in each peak are shown on the top RHS. In all profiles the relative proportions of peaks 3 and 4 (disialylated glycans) are reduced in cancer compared to peaks 1 and 2 (monosialylated glycans). In F4 there is a decrease in sialylation compared to F1-3. There is a decrease in the levels of F3 which contains both mono and di-sialylated glycans going from benign prostate hyperplasia to localised, locally advanced and metastatic prostate cancer. There is an increase in the levels of F4 which contains mostly mono-sialylated glycans going from benign prostate hyperplasia to localised, locally advanced and metastatic prostate cancer.

FIG. 25. N-Glycan analysis of alpha 1 acid glycoprotein excised from 2D gels of serum from healthy controls, patients with non-metastatic cancer and with metastasis pancreatic cancer. The results in these figures demonstrate that some glycans decrease with disease severity (shaded in FIG. 25 (a)) and some glycans increase with disease severity (shaded in FIG. 25 (b)). Typically the glycans contain SLex and higher branching.

FIG. 26 Shows Partial Least Squares—Disciminant Analysis (PLS DA) showing separation between patients with ovarian cancer and patients with benign tissue in the top charts, and separation between borderline patients and ovarian cancer patients in the lower charts. The analysis is based on the pooling of data from a number of glycosylation markers by PLS-DA, each marker pooled is indicated by numbered columns opposite the linked PLS-DA plot. Glycosylation marker F(β)A2 is indicated as column 2.

FIG. 27 HPLC analysis of the glycan pools on which the PLS-DA plot of FIG. 26 is based. The numbered peaks correspond to the numbers for each marker shown in the bar charts on the right hand side of FIG. 26.

### DETAILED DESCRIPTION OF THE INVENTION

Glycosylation of various proteins is altered in certain diseases and conditions, including cancer and chronic inflammation. A variety of novel glycosylation markers for diagnosing, treating, or monitoring cancer and/or inflammation are described herein. Methods employing the glycan markers are described, as are related compositions, systems and kits.

### Antibodies

Antibodies, e.g., antibodies specific for polypeptides bearing glycan markers of the invention, can be generated by methods well known in the art. Such antibodies can include, but are not limited to, polyclonal, monoclonal, chimeric, humanized, single chain, Fab fragments and fragments produced by a Fab expression library.

Polypeptides do not require biological activity for antibody production. However, the polypeptide or oligopeptide is antigenic. Peptides used to induce specific antibodies typically have an amino acid sequence of at least about 5 amino acids, and often at least 10 or 20 amino acids. Short stretches of a polypeptide can optionally be fused with another protein, such as keyhole limpet hemocyanin, and antibodies produced against the fusion protein or polypeptide.

Numerous methods for producing polyclonal and monoclonal antibodies are known to those of skill in the art, and can be adapted to produce antibodies specific for polypeptides bearing markers of the invention. See, e.g., Coligan (1991) Current Protocols in Immunology Wiley/Greene, N.Y.; and Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; Fundamental Immunology, e.g., 4th Edition (or later), W. E. Paul (ed.), Raven Press, N.Y. (1998); and Kohler and Milstein (1975) Nature 256: 495-497. Other suitable techniques for antibody preparation include selection of libraries of recombinant antibodies in phage or similar vectors. See, Huse et al. (1989) Science 246: 1275-1281; and Ward, et al. (1989) Nature 341: 544-546. Additional details on antibody production and engineering techniques can be found in U.S. Pat. No. 5,482,856, Borrebaeck (ed) (1995) Antibody Engineering, 2nd Edition Freeman and Company, NY (Borrebaeck); McCafferty et al. (1996) Antibody Engineering, A Practical Approach IRL at Oxford Press, Oxford, England (McCafferty), Paul (1995) Antibody Engineering Protocols Humana Press, Towata, N.J. (Paul), Ostberg et al. (1983) Hybridoma 2: 361-367, Ostberg, U.S. Pat. No. 4,634,664, and Engelman et al. U.S. Pat. No. 4,634,666. Specific monoclonal and polyclonal antibodies and antisera will usually bind with a Kd of at least about 0.1 μM, preferably at least about 0.01 μM or better, and most typically and preferably, 0.001 μM or better.

Molecular Biological Techniques

In practicing the present invention, many conventional techniques in molecular biology, microbiology, and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2000 and Current Protocols in Molecular Biology, F. M. Ausubel et al., eds., Current Protocols, a joint venture between Greene Publishing Associates, Inc. and John Wiley & Sons, Inc., (supplemented through 2007). Other useful references, e.g. for cell isolation and culture include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla. Methods of making nucleic acids (e.g., by in vitro amplification, purification from cells, or chemical synthesis), methods for manipulating nucleic acids (e.g., site-directed mutagenesis, by restriction enzyme digestion, ligation, etc.), and various vectors, cell lines and the like useful in manipulating and making nucleic acids are described in the above references. In addition, essentially any polynucleotide can be custom or standard ordered from any of a variety of commercial sources.

In addition to other references noted herein, a variety of purification/protein purification methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2nd Edition Wiley-Liss, NY; Walker (1996) The Protein Protocols Handbook Humana Press, N.J.; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, N.J.; and the references cited therein.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### Lung Cancer

Materials and Methods

Serum Sample Sets

Lung cancer serum samples used for the study were from patients diagnosed with lung cancer of non-small cell or small cell carcinoma lineages. Patient sera were examined alongside age-matched healthy control sera. Sera examined were from both male and female patients/volunteers. Lung cancer sera were obtained from Fox Chase, Cancer Center, Philadelphia, USA. Breast cancer patient sera were received from Prof. John Robertson (Breast Surgery Unit, Nottingham City Hospital).

One-Step Isolation of Haptoglobin from Serum

An affinity resin was prepared using mouse anti-human haptoglobin HG36 clone (H6395 Sigma-Aldrich). IgG was purified using a 1 ml HiTrap protein G column (Pharmacia) as previously described (Arnold J. N. et al.). The purified IgG (1 mg) was dialyzed into 0.1M NaHCO₃, 0.5M NaCl, pH8.3. An affinity resin was prepared using 0.29 g of cyanogen bromide activated Sepharose 4B (Sigma-Aldrich C9142) per ml of hydrated resin which was hydrated with 50 ml of 1 mM HCl for 15 min at RT. The HCl was filtered off and the 1 ml of moist resin cake was added to the dialyzed anti-haptoglobin IgG (0.5 mg/ml). This was stirred by slow rotation for 2 h at RT. The resin was washed with 20 ml of 0.1M Tris, 140 mM NaCl, pH8.0 and brought up in 30 ml of wash buffer and mixed by rotating for 2 h at RT to block any remaining active sites. The resin was then equilibrated in PBS-0.5 mM EDTA for storage.

Haptoglobin was purified from 20 μl of serum diluted to 1 ml with 10 mM Hepes, 1M NaCl, 5 mM EDTA, pH 7.4. This was then incubated with 10 μl (packed volume) of anti-haptoglobin-Sepharose resin and left at 4° C. for 1 hour at slow rotation for binding. The resin was removed through centrifugation at 1000×g, and washed twice by resuspension in 1 ml of dilution buffer followed by centrifugation as before. The pellet was dissolved in 5 μl Laemmli buffer (Laemmli.et al.) and 5 μl DTT (0.5M) and incubated for 5 mins at 70° C. before being loaded directly onto a 4-12% Bis-Tris gel (Invitrogen, US) for SDS PAGE analysis. Resolved proteins were visualised using Coomassie Blue stain.

Removal of N-linked Glycans from Serum and Sensitivity of Detection

Serum glycans were released from serum samples (10 μl) using the in-gel block method described by Royle et al., (Royle L. et al. (2006) Methods Mol Biol, 347, 125-143, Royle L et al. (2008) Analytical Biochem, 376, 1-12) or protein bands were excised from SDS PAGE. The N-linked glycans were released using the in-gel N-glycan release using peptide N-glycanase F (1000 units/ml; glycopeptidase, EC 3.5.1.52) as described previously (Bigge, J. C. et al. (1995) Anal Biochem, 230, 229-238, Kuster B. et al. Anal Biochem 1997; 250:82-101).

2-Aminobenzamide (2-AB) Labeling of Glycans

Released glycans were labelled by reductive amination with the fluorophore 2AB (Bigge, J. C. et al. (1995) Anal Biochem, 230, 229-238), using a Ludger Tag™ 2AB glycan labelling kit (Ludger Ltd, Oxford, UK).

Normal Phase (NP)HPLC and Weak Anion Exchange HPLC

Labelled glycans were separated on NP-HPLC (Guile G. R. Anal Biochem 1996; 240:210-26) and Weak Anion Exchange (WAX) HPLC. Glycan profiles from NP-HPLC were calibrated against a dextran ladder prepared from hydrolyzed and 2AB-labelled glucose oligomers (Guile G. R. Anal Biochem 1996; 240: 210-26). Glycans were assigned glucose units (GU) values and glycan structure/composition was predicted by reference to a glycan database (Glycobase http://glycobase.ucd.ie/cgi-bin/public/glycobase.cgi). Peak areas were established blind of the IL data to ensure fairness of test. WAX HPLC was conducted as described by Zamze et al. (Zamze S. et al. Eur J Biochem 1998; 258: 243-70) using a Vydac 301VHP575 7.5×50-mm weak anion exchange column (Hichrom, Berkshire, U.K.).

Exoglycosidase Digestions

Exoglycosidases were used to confirm the structures of glycans present in the preparations in conjunction with NP-HPLC (Radcliffe C. M. et al. J Biol Chem 2002; 277: 46415-23). Enzymes were used at the manufacturers' recommended concentrations and digests were carried out using 50 mM sodium acetate buffer, pH 5.5 for 16 hours at 37° C. Enzymes were supplied by Glyko Inc (Upper Heyford, UK); Arthobacter ureafaciens sialidase (ABS, EC3.2.1.18) 1-2 U/ml; almond meal α-fucosidase (AMF, EC 3.2.1.51), 3 mU/ml; bovine testis β-galactosidase (BTG, EC 3.2.1.23), 1 U/ml; jack bean α-mannosidase (JBM, EC 3.2.1.24), 100 mU/ml; bovine kidney fucosidase (BKF) (EC 3.2.1.51) 100 U/ml.

Serum Cytokine Levels

Two serum samples from patients with stage 4 lung cancer and two stage 4 breast cancer patients, identified to have elevated tri- and tetra-antennary structure and α1,3 fucose based on the serum glycosylation profiles, were analysed alongside control sera identified to have glycan profiles within normal ranges. The cytokine quantification was carried out as a service by ENDOGEN SEARCHLIGHT™ (Pierce Biotechnology, www.endogen.com). Pooled human serum came from citrated plasma (HDS Supplies, High Wycombe, UK) as described (Arnold J N, et al. J Biol Chem 2005; 280: 29080-7), the dilution of the citrate was corrected in the final IL level.

Cluster Analysis

For cluster analysis of immunochemical parameters investigated, the modified method of cluster analysis (Cluster—Statistica 5.0, Statsoft Inc., USA) through series of individual parameters was used. The joining tree clustering was carried out from the dataset of correlative measures of second order (r). Correlation coefficients were introduced to cluster analysis by using Chebyshev's distances as a measure of relatedness to exclude the negative meaning of r (S):

S(X,Y)=Maximum|Xᵢ−Yⱼ|

The value S can be considered as a measure of distance between the vectors and a measure of interrelations between immunochemical parameters investigated. In this case, the highest value of S is the smallest. In dendogram plots, the clusters of parameters are separated by levels of linkage (by method of average links of suspended grouping). Such clustering reflects the relatedness of certain parameters inside the whole spectrum of chemokines and cytokines involved in the study.

Statistics

The Shapiro-Wilk W test was carried out to determine normality of data distribution in each group. A two-tailed Mann-Whitney U-test was used for comparison of data between non-normally distributed groups, and Student's two-tailed t-test for independent groups was applied in cases of normal distribution. Spearman's rank correlation and Pearson's correlation were applied for appropriate correlation analyses. Statistics were performed using “Analyse-it Clinical Laboratory module” (Analyse-it Software Ltd., UK) and “Statistica-99 Edition” (Statsoft Inc., USA) software. Regression analysis was performed in Excel.

Results

Serum N-Linked Glycan Alterations in Lung Cancer Patients

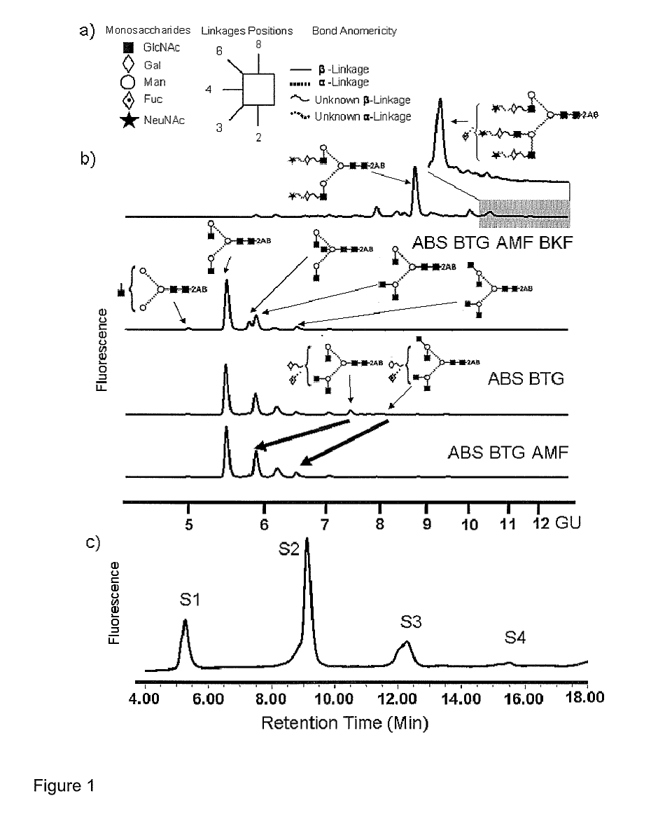

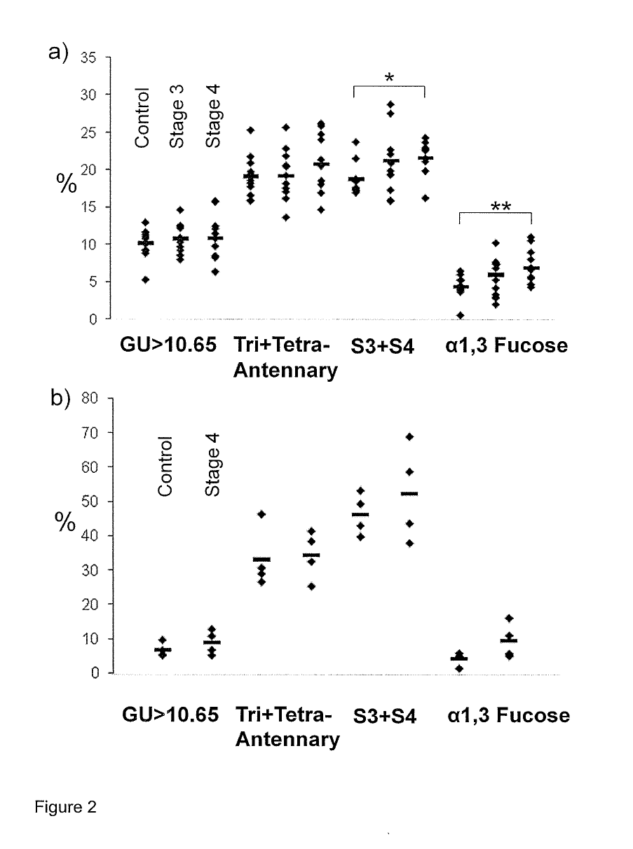

NP- and WAX HPLC, combined with exoglycosidase digestion, of the total serum N-linked glycome from lung cancer patients and healthy controls was carried out to identify and quantitate glycosylation changes (FIG. 1). The stage 4 lung cancer patients (n=12) had on average a statistically significant 15% increase in tri- and tetra-sialylated structures (p>0.05) and a 58% increase in α1,3 fucose (p>0.005) compared to healthy volunteers. The tri- and tetra-sialylated antennary glycans with α1,3 linked fucose predominantly elute at GU>10.65 on NP-HPLC (Royle L. et al. (2006) Methods Mol Biol, 347, 125-143, Royle L et al. (2008) Analytical Biochem, 376, 1-12.) (FIG. 1 highlighted area). The HPLC peak areas of sugars eluting with GU values >10.65 in the stage 4 cancer patients were increased on average 36% compared to the control population (FIG. 2) (p<0.012, t=2.91, df=13). Using exoglycosidase digestions, preliminary assignments were confirmed (FIG. 2). The stage 4 lung cancer patients (n=4) had on average a statistically significant 32% increase in sialylated tri- and tetra-antennary structures (p<0.001, t=4.21, df=13) and a 76% increase in α1,3 fucose (p<0.005, t=3.33, df=13). The stage 3 lung cancer patients (n=7) had no significant alterations in the level of sialylation, branching or α1,3 fucose compared to the healthy controls. The individual spread of the data between the groups had considerable overlap, these make differentiation of individual samples solely based on these glycosylation changes difficult (FIG. 2).

Haptoglobin N-Linked Glycosylation Changes in Lung Cancer

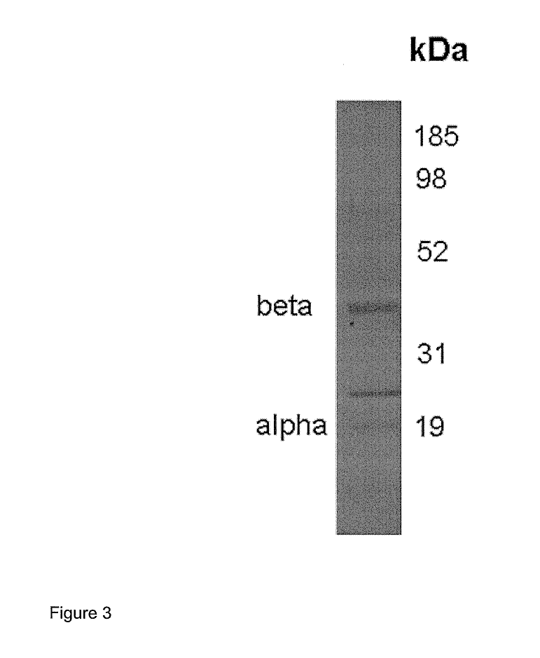

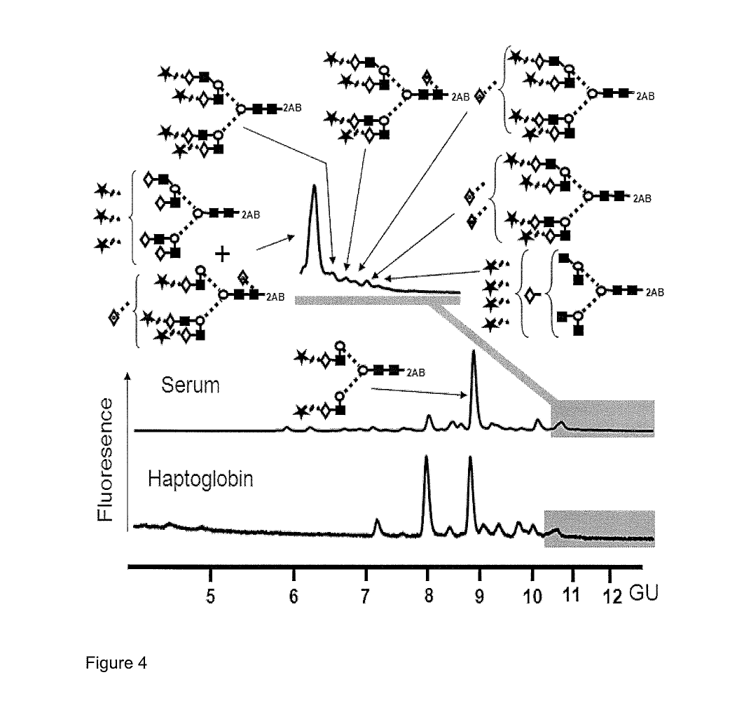

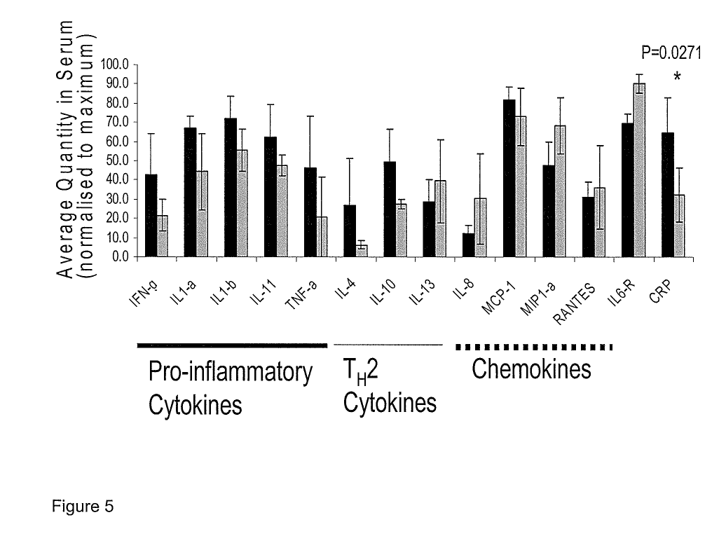

Haptoglobin, which circulates at approximately 1-2 mg/ml in the serum, was isolated from stage 4 lung cancer patients (n=4) and age matched controls (n=4) (FIG. 3) and the N-linked glycans of the beta chain were released (FIG. 4). This was carried out to demonstrate that the glycosylation changes identified in the stage 4 lung cancer set were not solely the result of an increase in the level of the acute phase proteins, but a shift in the glycoform population attached to these proteins. The haptoglobin glycan pool, on average, had a 32% increase in glycan structures with GU values >10.65 (FIGS. 2b and 4) (p>0.331, t=1.06, df=β). When the glycan pool was analysed for the level of α1,3 fucose attached to haptoglobin of the patient group presented a 120% increase compared to the control group (p>0.111, t=1.87, df=β). Due to the small sample set the data did not reach statistical significance, however, these data demonstrated a shift in the glycoform population of the haptoglobin in serum of cancer patients (FIG. 2).

Analysis of the Serum Cytokine Data

This study identified that in lung cancer some stage 4 patients have increases in their serum levels of sialylated tri- and tetra-antennary structures with or without α1,3 fucose residues. Sera of patients (n=4) and controls (n=4) were screened for a panel of cytokines. Large variations of cytokine levels were observed between individuals. On average the cancer patients presented higher levels of the pro-inflammatory cytokines (FIG. 5). Also the T_{H}2 cytokines IL-4 and IL-10 levels were on average modestly increased in the patients. sIL-6R, the soluble form of the IL-βR, was reduced in patient serum. The reduction of sIL-6R was statistically significant (P<0.027, t=2.91, df=β). The other marker of inflammation, CRP, was on average modestly increased in the patient group.

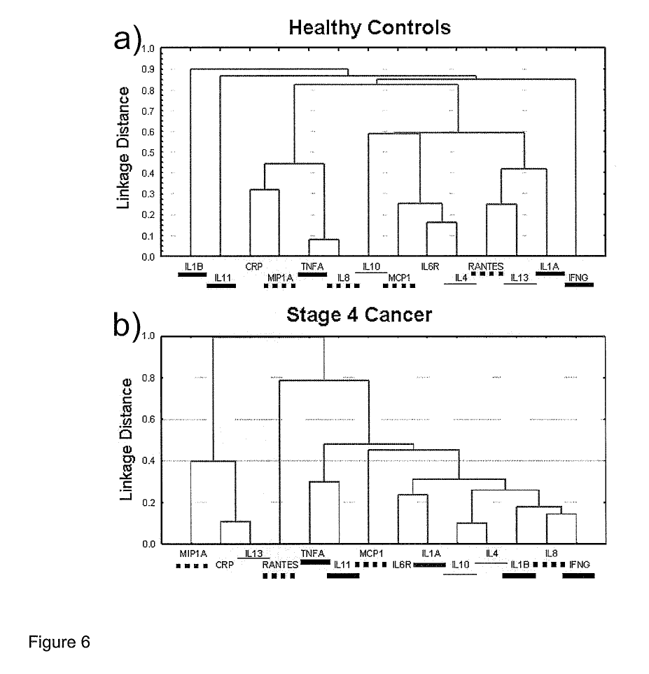

A different statistically significant set of correlations (p<0.05) was identified between the expression levels of individual cytokines in the serum of the patient and control group (Table 1). Cluster analysis was used to characterize the overall interplay (or relatedness) between the individual cytokine levels within both groups. The analysis identified a strong linkage between the pro-inflammatory cytokines in the patient group, while there was almost no linkage between the inflammatory cytokines in the control (FIG. 6). Interestingly, in the patient group, the T_{H}2 cytokines IL-4 and IL-10 and the chemokine MCP-1 were related to the pro-inflammatory cytokines, where the T_{H}2 cytokines IL-10 and IL-4 were closely linked within the pro-inflammatory cytokine cluster (FIG. 6). These data indicate that the interplay of cytokines is different between the control and the stage 4 cancer set. The cytokine levels and the cluster profile of the cancer patient cytokine data reflects an inflammatory state in the stage 4 cancer patients (FIGS. 5 and 6). This study also indicates additional cytokine candidates that may modulate the glycosylation changes in cancer.

Table 1 presents the analysis to identify correlations between individual cytokines (n=4) for a) healthy controls and b) stage 4 cancer patients. Boxed are the statistically significant correlations (p<0.05). Also shown are the correlation coefficients, which were used to carry out the cluster analysis. The chemokines MCP-1, MIP-1α and Rantes are also known as CCL2, CCL3 and CCL5.

TABLE 1
Correlations between individual cytokines for a) healthy controls and b) stage 4 cancer patients.
a) Healthy Controls

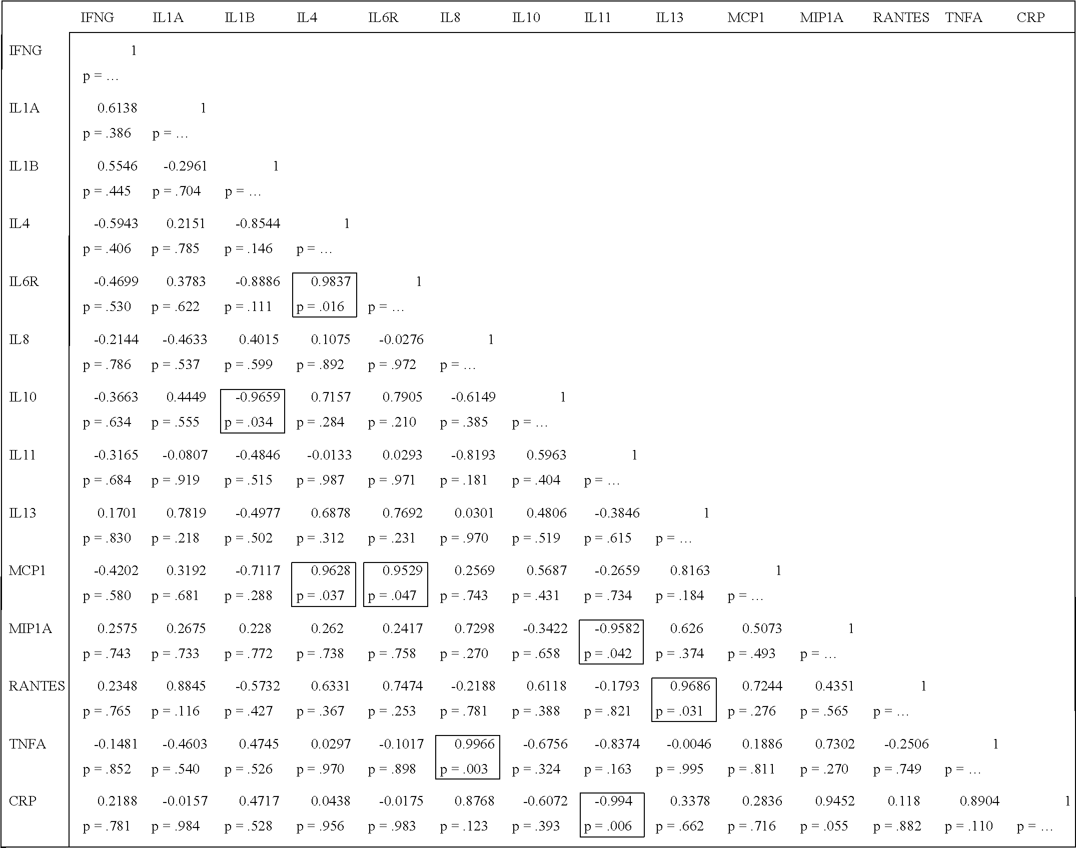

b) Stage 4 Cancer

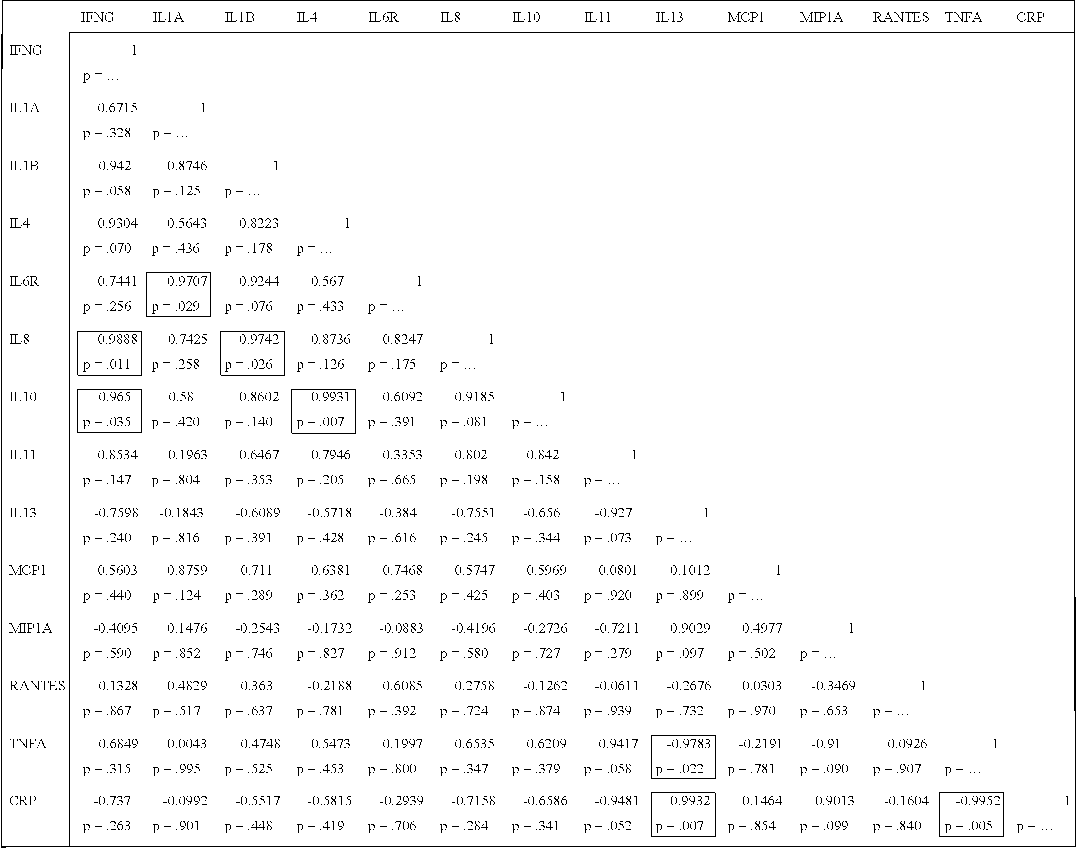

Correlation of Cytokine Data and CRP Levels with Glycosylation Data

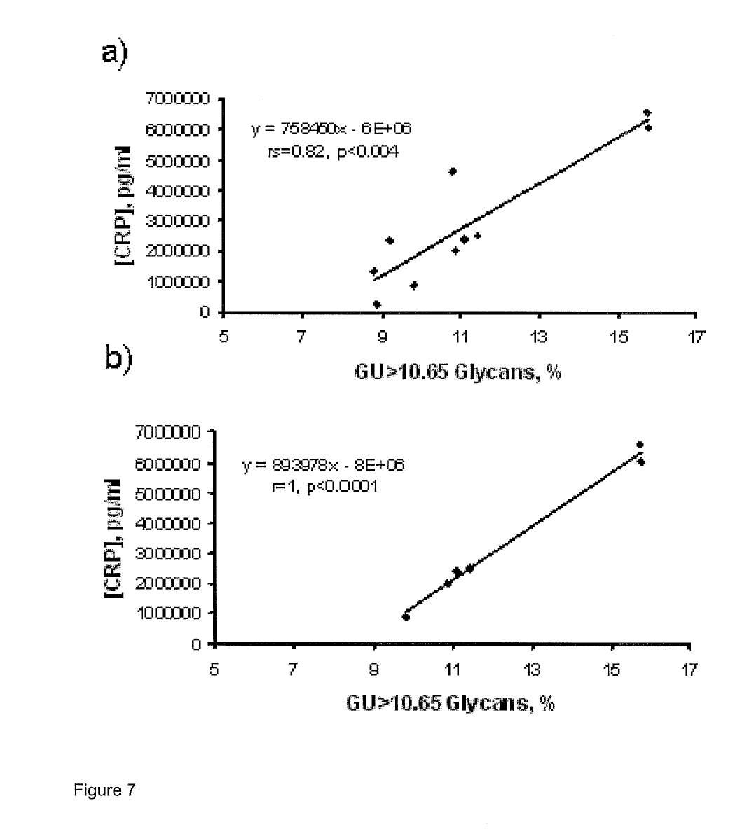

The cytokine data from the stage 4 cancer patients and their controls was analysed against the glycosylation data to identify any significant correlations. The percentage of glycan structures with GU values >10.65 (where predominantly sialylated tri- and tetra-antennary structures with or without α1,3 fucose elute) did not correlate with any of the cytokines analysed. A statistically significant positive correlation (rs=0.82, p<0.004) was identified between CRP and the total percentage of glycans with GU values >10.65 (FIG. 7a). CRP did not correlate with the level of tri- and tetra-antennary structures (r=0.61, p<0.063), but did correlate with the level of α1,3 fucose (r=0.78, p<0.008). The patient CRP concentrations, when analysed separately from the controls had an almost perfect linear arrangement when correlated with the GU values >10.65 (Pearson's correlation r=1, p<0.0001) (FIG. 7b). The controls when analysed separately did not show a statistically significant correlation with CRP (r=0.94, p>0.056).

Discussion

Using a lung cancer sample set and healthy controls, fully quantitated glycan analysis using HPLC separation of released glycans from both the serum glycome and from isolated haptoglobin is presented. Increases in sialylated tri- and tetra-antennary structures with α1,3 linked fucose were identified in the serum N-linked glycome from stage 4 lung cancer patients (n=12). Two stage 4 lung cancer sera and two stage 4 breast cancer sera were also screened for an array of cytokines. This was performed to identify correlations between the glycosylation data and the cytokine data to identify potential markers and further candidate cytokines which could influence glycosylation in cancer/chronic inflammation. The cytokine data demonstrated, as predicted, that serum from cancer patients contained inflammatory markers. The glycosylation data did not correlate with the cytokine data obtained. However, the percentage of multi-antennary larger glycan structures with GU values >10.65 had a statistically significant correlation with serum CRP.

Glycosylation Changes in Stage 4 Lung Cancer Patients

The serum glycome (117 unique structures) has been fully characterised previously using HPLC data in combination with mass spectrometry analysis (Royle L. et al. (2006) Methods Mol Biol, 347, 125-143, Royle L et al. (2008) Analytical Biochem, 376, 1-12). The glycosylation changes in a lung cancer sample set were quantitated. In stage 4 lung cancer, a significant increase in α1,3 fucose and sialylated tri- and tetra-antennary structures was identified (FIG. 2). Consistent with these findings, the serum glycome showed an increase in the glycan structures with GU values >10.65 (FIG. 2). These are predominantly sialylated tri- and tetra-antennary glycans with or without α1,3 fucose (FIGS. 1 and 2) (Royle L. et al. (2006) Methods Mol Biol, 347, 125-143, Royle L et al. (2008) Analytical Biochem, 376, 1-12). The N-linked glycans of isolated haptoglobin (FIG. 4) demonstrated that the changes identified at the serum glycome level were, in part, caused by shifts in the glycoform population and not solely increases in the serum concentrations of the acute phase proteins (FIG. 2b). In agreement with these results, 68% of the haptoglobin isolated from stage 3 and stage 4 pancreatic cancer patients was also found to have statistically elevated fucosylation.

Analysis of the Cytokine Data in Cancer Versus Control and its Correlation with Glycosylation Changes in Cancer

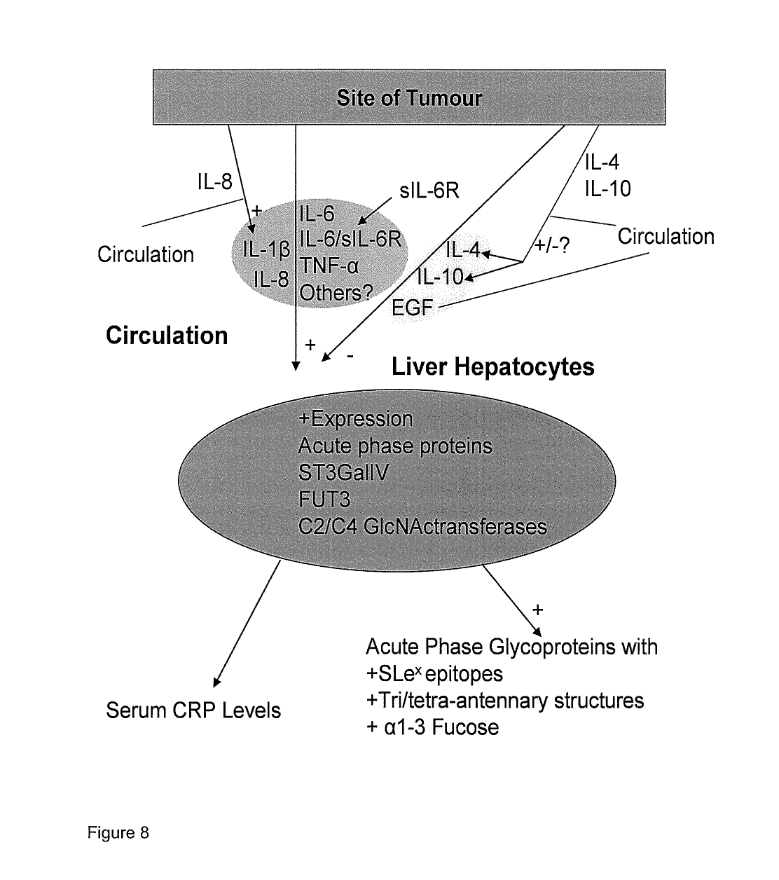

Serum from four stage 4 cancer patients and four healthy controls was analysed for a selection of cytokines to identify cytokines that may be implicated in alterations to the serum N-linked glycome. An average increase was identified in the pro-inflammatory cytokines in the cancer patients (FIG. 5). The control group showed minimal linkage between the cytokines (FIG. 6), however, the cancer group had a strong linkage between pro-inflammatory cytokines (FIG. 6). Taken together the cytokine data indicate that the stage 4 lung cancer patients are generate an inflammatory response as a result of the tumour. IL-1, IL-6 and TNF-a have shown to stimulate hepatocytes to secrete the acute phase proteins (FIG. 8). Serum IL-1 and TNF-a were on average modestly increased in the cancer group (FIG. 5). In the NCl-H292 carcinoma cell line, TNF-α increases the selective expression of the ST3GaIIV, FUT3 and C2/C4 GlcNAc transferases (which forms tri- and tetra-antennary structures) (Ishibashi Y. et al.). There was a significant correlation between IL-8 and IL1β(r=0.97, p<0.026) (Table I), consistent with previous findings which have demonstrated that IL1β induces transcriptional activation of the IL-8 gene. The biological activity of IL-6 is mediated through two membrane bound proteins, a unique low affinity binding receptor IL-βR and the high affinity receptor gp130. IL-βR acts as an agonist to IL-β. IL-6 complexed with sILβR can activate cells by binding to the cell surface receptor gp130. Soluble forms of the cytokine receptors are found in vivo because of alternative splicing of the mRNA and as a result of proteolysis (shedding) of the membrane bound receptor. In several conditions such as HIV infection multiple myeloma, juvenile arthritis, Crohn's disease and ulcerative colitis elevated levels of sIL-6R have been observed. sILβR has been implicated in the modulation of the liver response in acute and chronic infection by increasing the production of the acute phase proteins α1-anti-chymotrypsin and haptoglobin through promotion of the hepatocyte response to IL-6 in a dose and time dependent manner. The levels of free sIL-6R in the cancer group were reduced (p<0.027) (FIG. 5). The assay used to detect sIL-6R utilises an antibody raised against free sIL-6R and as such is unlikely to detect sIL-6R in complex with IL-6. In the inflammatory response the serum levels of IL-6 increase, this will result in higher levels of IL-6 in complex with sIL-βR, lowering the amount of free sIL-6R in the serum (FIG. 7a). It was demonstrated that sIL-6R had a statistically significant correlation with the anti-inflammatory cytokine IL-4 (r=0.98, p<0.016) in the control group and correlated with the pro-inflammatory cytokine IL-1α in the patient group (r=0.97, p<0.029), these two associations of cytokine with sIL-6R are closely related on the cluster diagram respectively, possibly suggesting a degree of sIL-6R modulation by these cytokines (FIG. 6).

IL-4 inhibits the induction of some cytokine-induced acute phase proteins from hepatocytes as does EGF. The data suggest that IL-4 is increased in the cancer group (FIG. 4) and is also linked with the pro-inflammatory cytokines modulating the inflammatory response (FIGS. 6 and 8). There is a significant correlation between the anti-inflammatory cytokines IL-10 and IL-4 in the cancer group (rs=0.95, p<0.005) and a strong linkage on the cluster analysis (FIG. 6 and Table 1). These data suggest that the alterations of these cytokines are closely related and may be modulating each other. There was no statistically significant correlation between any of the cytokines and the glycosylation data. The cytokine data are not directly linked to the glycosylation data, possibly because of the cross-modulating (combined) effects of these molecules. The glycosylation changes in inflammation arise from several cytokines, having both effecter functions individually and in cohort (FIG. 8).

Inflammatory Marker CRP Correlates with the Percentage of Serum N-Linked Glycans with GU Values >10.65

A significant correlation was identified between CRP and the percentage of structures with GU values >10.65 (p<0.004) and percentage α1,3 fucose (p<0.008), but not the level of tri- and tetra-antennary structures. CRP is a non-specific serum marker for inflammation. CRP levels above baseline have been linked to a risk of developing colon cancer, but not rectal or prostate. CRP is not present in the serum without an inflammatory response, and is only expressed in the liver during inflammation. When analysing CRP for linkage to the inflammatory cytokines it was demonstrated that in the patient group CRP was not linked to the pro-inflammatory cytokines (FIG. 6). The serum concentration of CRP is a down-stream result of multiple cytokines acting in combination to elicit a refined acute phase response. For example, IL-4 has been demonstrated to be able to down regulate the production of CRP but not fibrinogen or α1-anti-trypsin and can inhibit IL-6 induced expression of haptoglobin but not CRP. IL-8 was highly related to the pro-inflammatory cytokines in the patient group (FIG. 6), and has previously been demonstrated to promote the production of CRP from hepatocytes (Wigmore S. J. et al. Am J Physiol 1997; 273:720-β). The correlation of CRP and percentage of structures with GU values >10.65 correlated in an almost perfect linear arrangement when analysed as the patient group separately (r=1, p<0.0001). The control group showed no correlation when analysed alone (r=0.94, p>0.056). These data demonstrate that ‘long term’ chronic inflammation results in a pronounced alteration in the serum glycoform population.

Summary

In conclusion, the serum N-linked glycosylation changes in a lung cancer group have been identified using quantitative NP-HPLC and WAX methods. The serum samples were screened for a panel of cytokines and it was demonstrated that the serum glycosylation changes in cancer relate to an inflammatory state of the serum based upon cytokine analysis. Using the quantitative aspect of the glycosylation analysis method employed in this study, it was attempted to correlate the glycosylation data to serum cytokine levels. The N-linked glycosylation changes in cancer do not correlate with the serum level of any single cytokine analysed in the panel, however, the percentage of glycans with GU values >10.65 surprisingly correlated with the level of serum inflammation marker CRP (FIG. 7a). This correlation is almost perfectly linear when analysed as the patient group alone (FIG. 7b), suggesting that the glycosylation changes (specifically percentage of glycan structures with GU levels >10.65) seen in cancer patients may be directly linked to the inflammatory state of the patient. The glycosylation changes are specific to chronic inflammation, such as in cancer (FIG. 2). Serum CRP levels do not discriminate between chronic and acute inflammation, demonstrated in the absence of a correlation between CRP and the serum glycans with GU>10.65 in the control group. This suggests that the analysis of glycosylation changes such as percentage of glycans with GU values >10.65 may represent a more specific cancer diagnostic than CRP.

### Example 2

### Breast Cancer

Materials and Methods

Serum Samples

Serum were obtained from cancer-free female controls (n=19) and advanced breast cancer patients (n=18) in the Breast Surgery Unit, Nottingham City Hospital with informed consent prior to sample collection. The average age for the cancer-free women was 42±13 years, compared with 63±13 years for the breast cancer patients. From the same sample bank, we received four serum samples from Patient A for a longitudinal study. An additional pooled control comprising of serum from over 30 individuals was obtained from The National Health Service (NHS) as analysed in Royle et al. (Royle L. et al. (2006) Methods Mol Biol, 347, 125-143, Royle L et al. (2008) Analytical Biochem, 376, 1-12).

N-Glycan Release by the In-Gel Block Method

Serum samples (5 ul) were subjected to the In-gel block method as previously described (Royle L. et al. (2006) Methods Mol Biol, 347, 125-143). Briefly, N-glycans were released from serum gel blocks or protein spots excised from 2D gels of serum by PNGaseF digestion (100 U/ml, EC 3.5.1.52, Roche Diagnostics GmbH, Mannheim, Germany) carried out at 37° C. for 18 hours. The extracted glycan pool was then subjected to 2AB fluorescent labelling using the Ludger Tag™2AB kit (Ludger Ltd, Oxford, UK).

Glycan Analysis by HPLC and Mass Spectrometry

The labelled N-glycans were subsequently analysed by Normal Phase (NP)HPLC using a TSK gel Amide-80 column with a 20-58% gradient of 50 mM ammonium formate pH 4.4 vs acetonitrile. The system was calibrated using an external standard of hydrolysed and 2AB-labelled glucose oligomers which forms a dextran ladder. Weak anion exchange (WAX) HPLC analysis of the N-glycans was carried out using a Vydac 301VHP575 7.5×50 mm column (Royle L. et al. (2006) Methods Mol Biol, 347, 125-143).

MALDI-TOF Mass Spectrometry

Positive ion MALDI-TOF mass spectra were recorded with a Micromass TofSpec 2E reflectron—TOF mass spectrometer (Micromass, Manchester, United Kingdom) fitted with delayed extraction and a nitrogen laser (337 nm). The acceleration voltage was 20 kV; the pulse voltage was 3200 V; the delay for the delayed extraction ion source was 500 ns. Samples were prepared by adding 0.5 μl of an aqueous solution of unlabelled glycans to the matrix solution (0.3 ml of a saturated solution of 2,5-dihydroxybenzoic acid in acetonitrile) on the stainless steel target plate and allowed to dry at room temperature. The sample/matrix mixture was then recrystallized from ethanol (Harvey, D. J., Nat Methods, 2007).

Negative Ion Electrospray Ionisation Mass Spectrometry ESI-MS and ESI MS/MS

Nano-electrospray mass spectrometry was performed with a Waters-Micromass quadrupole-time-of-flight (Q-TOF) Ultima Global instrument. Unlabelled glycan samples in 1:1 (v:v) methanol:water containing 0.5 mM ammonium phosphate were infused through Proxeon (Proxeon Biosystems, Odense, Denmark) nanospray capillaries. The ion source conditions were: temperature, 120° C.; nitrogen flow 50 L/hour; infusion needle potential, 1.2 kV; cone voltage 100 V; RF-1 voltage 150 V. Spectra (2 sec scans) were acquired with a digitization rate of 4 GHz and accumulated until a satisfactory signal:noise ratio had been obtained. For MS/MS data acquisition, the parent ion was selected at low resolution (about 4 m/z mass window) to allow transmission of isotope peaks and fragmented with argon. The voltage on the collision cell was adjusted with mass and charge to give an even distribution of fragment ions across the mass scale. Typical values were 80-120 V.

Glycan Sequencing and Exoglycosidase Digestion

N-glycan structures were assigned glucose units (GU) by comparison to the retention time of a standard dextran ladder. Further sequencing and structure confirmation was based on sequential exoglycosidase digestions followed by NP HPLC (Royle L. et al. 2006). Labelled glycans were digested with an array of enzymes at manufacturer's recommended concentrations in 50 mM sodium acetate buffer pH 5.5 (or 100 mM sodium acetate, 2 mM Zn²⁺ pH 5.0 for JBM digestion) at 37° C. for 16 hours. The enzymes include Arthrobacter ureafaciens sialidase (ABS, EC 3.2.1.18), Bovine testis β-galactosidase (BTG, 3.2.1.23), Streptococcus pneumoniae β-galactosidase (SPG, EC 3.2.1.23), Almond meal α-fucosidase (AMF, EC 3.2.1.111), recombinant Streptococcus pneumonia hexosaminidase (GUH, EC 3.2.1.30), and Jack bean β-N-acetylhexosaminidase (JBH, EC 3.2.1.30) purchased from Prozyme (San Leandro, Calif., USA) and Glyko (Novato, Calif., USA).

Two-Dimensional Gel Electrophoresis of Breast Cancer Serum

2D separation of the pooled control and breast cancer patient serum sample was carried out in duplicates for both anti-SLe^{x} blotting and fluorescent staining. 80 ug of serum was used per gel based on the protein concentration determined by using the Bicinchoninic acid (BCA) assay method of Smith et al. (Smith P. K. et al. Anal Biochem, 1985. 150(1): p. 76-85). Each aliquot of serum was mixed with 5 M urea, 2 M thiourea, 4% (w/v) 3-[(3-cholamidopropyl)dimethylammonio]-1-propanesulfonate (CHAPS), 65 mM dithiothreitol (DTT), 2 mM tributyl phosphine (TBP), 150 mM NDSB-256 (dimethylbenzylammonium propane sulfonate, non-detergent sulfobetaine-256 —NDSB-256, Merck Biosciences Nottingham, UK) and 0.002% (w/v) bromophenol blue, 0.45% (v/v) of pH 2-4 carrier ampholytes (SERVALYT® SERVA, Heidelberg, Germany), 0.45% (v/v) of pH 9-11 carrier ampholytes and 0.9% (v/v) of pH 3-10 carrier ampholytes for a total volume of 120 μl per gel and transferred into reswelling trays. Immobiline® IPG DryStrip pH 3-10 NL, 7 cm (Amersham Biosciences) were placed face down onto the samples, covered with 1 ml of mineral oil and left overnight at room temperature to allow rehydration (Sanchez, J. C. et al. (1997) Electrophoresis, 18, 324-327).

Following this, the strips were transferred to the Multiphor II with the gel facing upwards and damp wicks placed on both ends. IEF was carried out at 300 V for 1 minute, 3500V for 90 minutes and then another 100 minutes at 3500 (Sanchez, J. C. et al. (1997) Electrophoresis, 18, 324-327). The IPG strips were then immediately equilibrated for 15 min in 4M urea, 2 mM thiourea, 12 mM DTT, 50 mM Tris (pH 6.8), 2% (w/v) SDS, 30% (w/v) glycerol at room temperature and placed on top of the second dimension 4-12% Bis-Tris Zoom™ (Invitrogen) gels embedded in 0.5% melted agarose. Second dimension electrophoresis was carried out at 125V for 2 hours. A gel from each sample was fixed in 40% (v/v) ethanol, 10% (v/v) acetic acid overnight and stained with the fluorescent dye OGT 1238 (Oxford Glycosciences, Abingdon, UK) according to Hassner et al. (Hassner A. (1984) Synthesis. J Org Chem, 49, 2546-2551). 8-bit monochrome fluorescent images were captured at using a FujiCCDC Camera LAS_{—}1000 plus (Tokyo, Japan).

N-Glycan Release, Peptide Extraction, LC-MS/MS and Data Analysis for Protein Identification

Protein features assigned to mass spectrometric analysis were excised manually. The recovered gel pieces were reduced with 0.5M DTT at 65° C. for 20 minutes followed by a 30 minute incubation in 100 mM IAA and an overnight digestion with PNGaseF to cleave the N-glycans, as described earlier. Following glycan extraction, the gel pieces were dried in a SpeedVac, and in-gel trypsin (Roche. Basel, Switzerland) digestion was carried according to the protocol of Shevchenko et al. (Shevchenko A. et al. Proc Natl Acad Sci USA, 1996. 93(25): p. 14440-5). The tryptic peptides were analyzed by liquid chromatography tandem mass spectrometry (LC-MS/MS) as previously described (Garcia, A. et al. Proteomics, 2004. 4(3): p. 656-68).

Immunoblotting

Proteins from 2D gels of 80 μg total serum proteins described previously were transferred to a nitrocellulose membrane by Western blotting.

Membranes were blocked with 0.2% I-Block (Tropix) in PBST for 1 hour at room temperature before an overnight incubation in 5 ug/ml KM93 (Calbiochem) in 0.02% blocking solution at 4° C. Membranes were washed with 0.5% PBST before 1 hour incubation with 0.5 pg/ml anti-mouse IgM (Sigma Aldrich). The blots were developed using chemiluminescent detection system (ECL Plus Amersham).

Results

The N-Glycan Pool of Advanced Breast Cancer Serum

N-linked glycans from total serum glycoproteins of advanced breast cancer patients (n=19) and cancer-free controls (n=18) were analysed by NP and WAX HPLC in combination with sequential digestion using an array of exoglycosidases and MS. 117 N-glycans were previously identified in control serum by these methods as described in Royle et al. (Royle L. et al. (2006) Methods Mol Biol, 347, 125-143, Royle L et al. (2008) Analytical Biochem, 376, 1-12) and Harvey et al. (Harvey D. J. 2007).

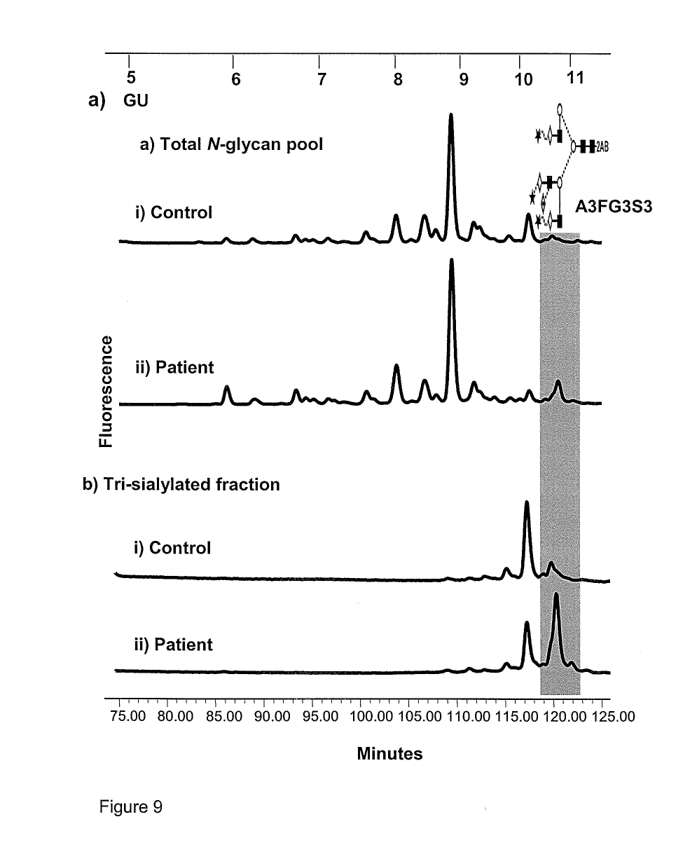

A comparison between breast cancer and control serum proteins N-glycans showed the breast cancer N-glycans to have increased amounts of outer arm fucosylation with the fucose α1,3 linked to the terminal GlcNAc on the tri-sialylated tri-antennary structure with GU value of 10.75 (A3FG3S3) (FIG. 9a). α1,3 linked fucose on the non-reducing terminus of A3G3S3 constitutes the SLe^{x} epitope, which is a ligand for E-selectin involved in leukocyte homing on endothelial cells.

Fractionation of the Glycan Pool Based on Total Charge (Degree of Sialylation) by WAX HPLC Followed by NP HPLC.

This method enables detailed comparison of structures from each differently charged fraction (with 0-4 sialic acid residues) and as shown in FIG. 9b, highlights that the increase in the α1,3 fucosylated tri-antennary is in the tri-sialylated fraction. Other N-glycosylation changes identified by HPLC and MS in the patient sera are increased levels of the less abundant structures including α1,3 difucosylated tri-antennary, α1,3 mono and difucosylated tetra-antennary, tetra-antennary glycans with lactosamine extensions and increased α2,3 compared to α2,6 sialylation (data not shown).

Sequencing and Quantification of the Glycan Marker, A3FG1

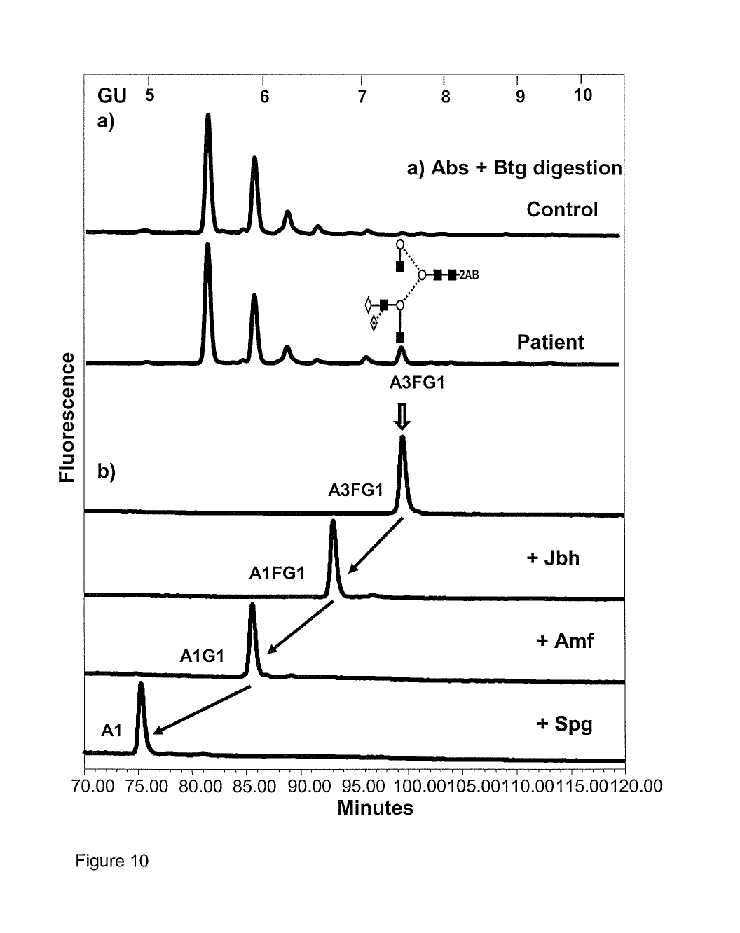

A set of exoglycosidase array digestions were performed to segregate and amplify the glycan structures, as well as to confirm specific linkages. Following a combination of sialidase and β-galactosidase, we were able to isolate the increased α1,3 fucosylated tri-sialylated tri-antennary structure (GU10.75) as it collapsed to form the α1,3 fucosylated monogalactosylated tri-antennary structure (A3FG1) at GU7.5 (FIG. 10a). The presence of an outer arm fucose hinders the cleavage of the galactose that is linked to the same GlcNAc by the galactosidase, resulting in the product, A3FG1.

As the linkage of the outer arm fucose and galactose (linked to the same GlcNAc) determines whether it is a sialylated Lewis x (α1,3 fucose, β1,4 galactose) or Lewis a (α-1,4 fucose, α1,3 galactose), it was crucial to distinguish the specific linkages of the glycan marker. Therefore, a combination of both α1,3/4 fucosidase and β1,4 galactosidase digest was performed on the glycan pool and was found to digest the A3FG1 peak completely, confirming the terminal epitope as a sialylated Lewis x. This was consistent with data obtained by ion fragmentation using Nanospray-CID mass spectrometry (data not shown).

Isolation of the peak at GU7.5 was performed to specify which GlcNAc the α1,3 fucose was linked to. Following a range of digestions, the GlcNAc which the fucose is linked to was shown to be the linked β1,4 to the tri-mannosyl core (FIG. 10b). The GU for this structure was confirmed by comparison with the known N-glycans of IgG as a standard.

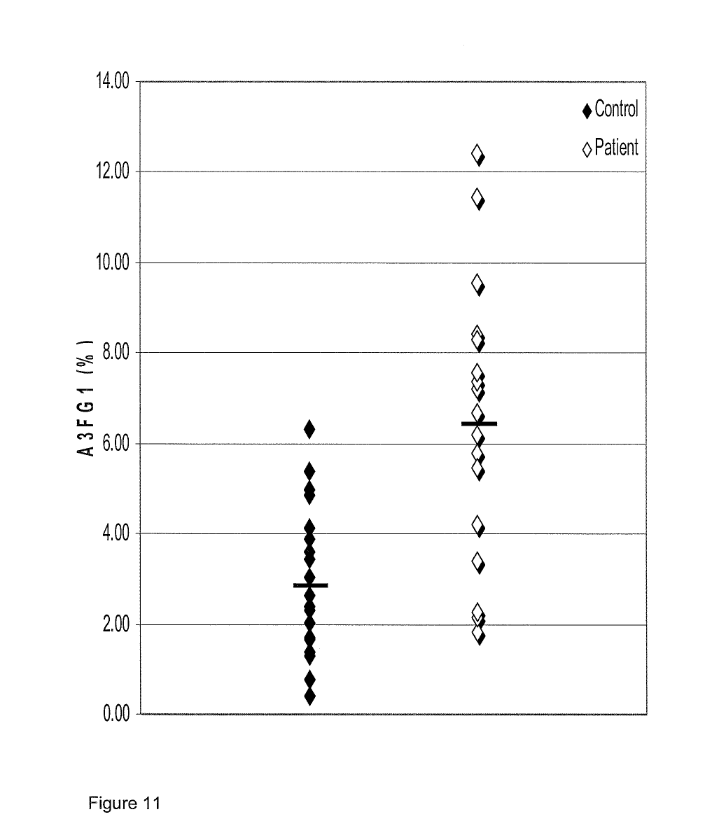

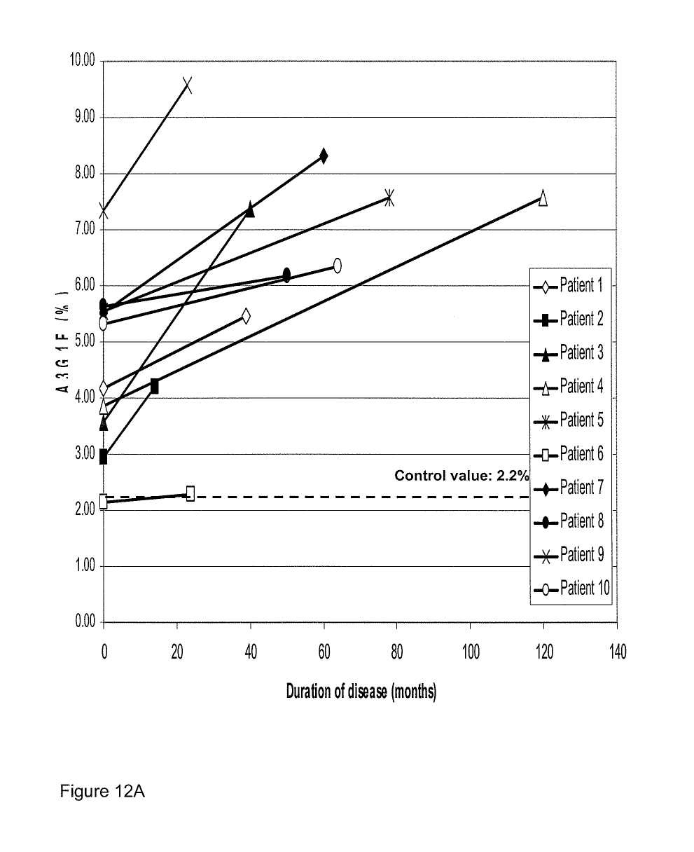

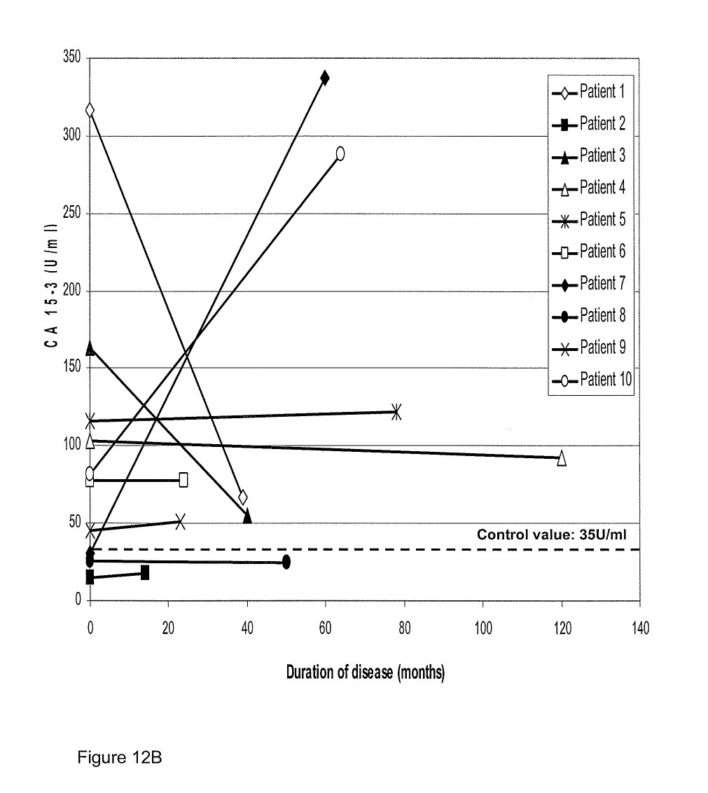

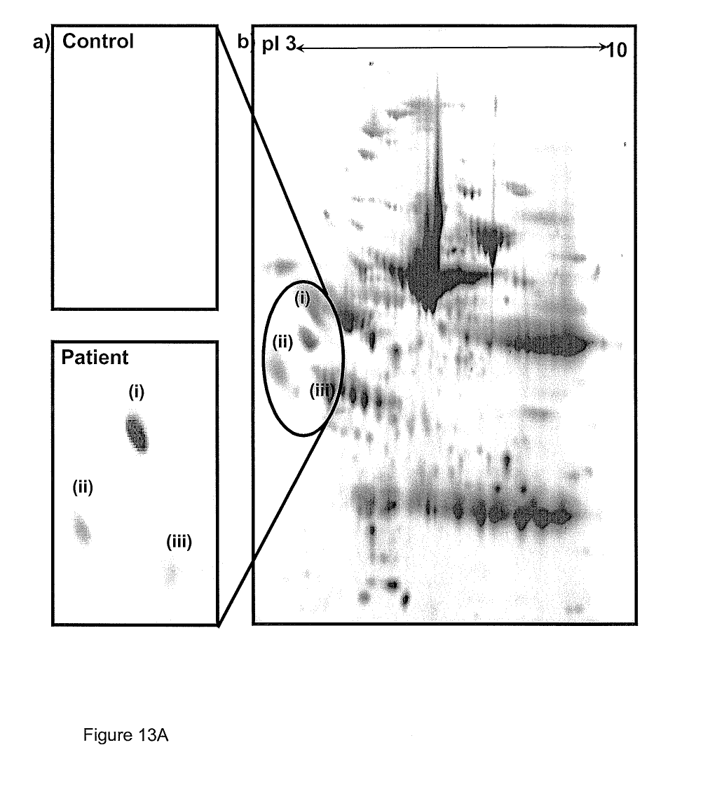

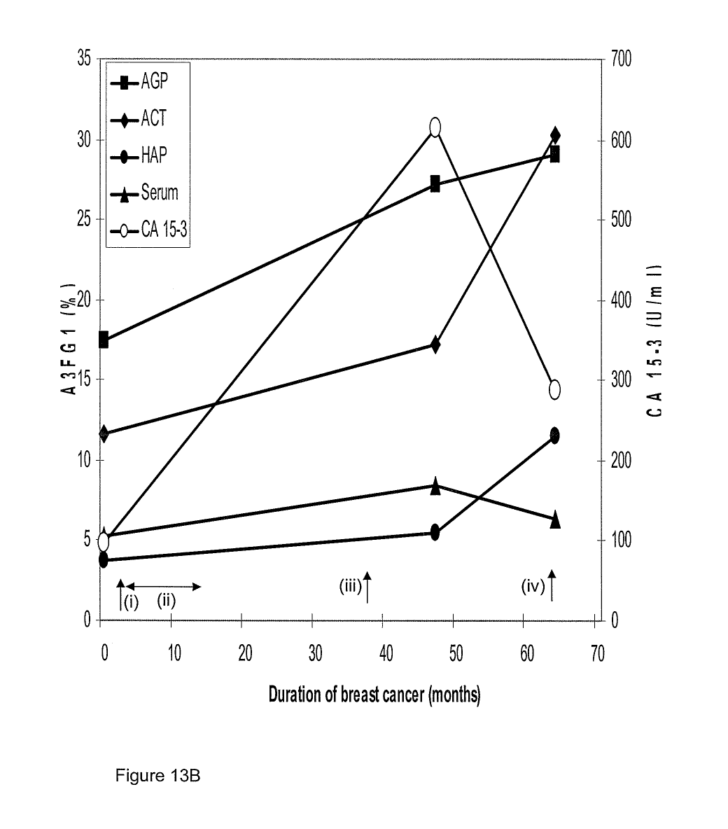

The percentage areas of the A3FG1 were quantified and compared against the total N-glycan pool in all breast cancer patients and controls. As shown in FIG. 11, there was a marked increase of approximately 3 fold in the average for the advanced breast cancer (6.55%±3.02) compared to control (2.96%±1.65).

A3FG1 as a Biomarker for Breast Cancer Progression

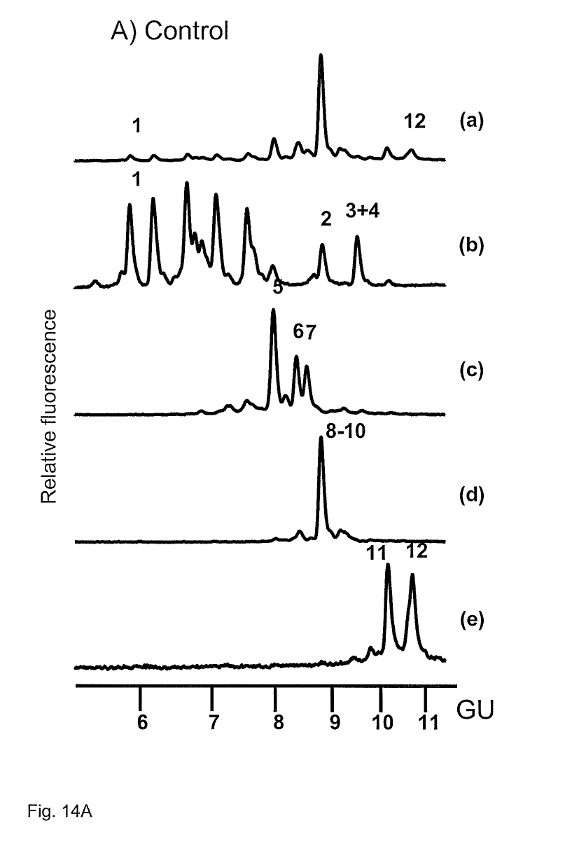

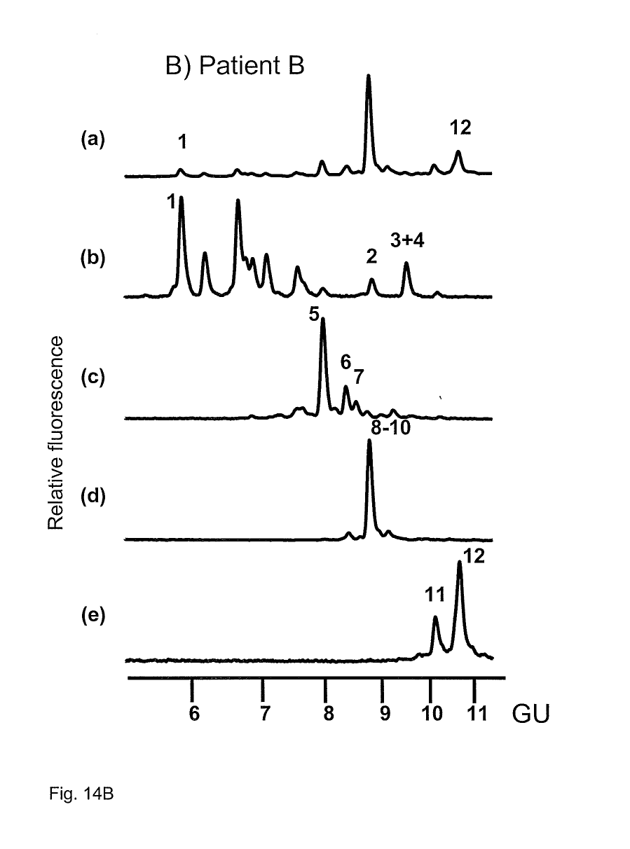

To evaluate the potential of A3FG1 as an indicator of breast cancer progression, a longitudinal case study was performed on ten individual patients (Patient A) where the levels of A3FG1 were plotted against CA 15-3 from serum collected at two time points during the malignancy, with the earlier sample taken when breast cancer was first diagnosed, and the later after metastasis was detected in each of them (FIG. 13B). A significant difference in the trends of both A3FG1 and CA 15-3 was observed in all ten patients. Interestingly, we found the A3FG1 increased in all the second samples, clearly indicating breast cancer progression. This was in contradiction with the CA 15-3 levels which out of ten patients, only showed increase levels in four cases, while the other showed no significant increase and two cases even had reduced levels. This suggests that compared to the commonly measure CA 15-3, the glycan marker A3FG1, measured from whole serum of breast cancer patients, is more reliable in detecting disease progression and metastasis.

Glycoproteomics Approach to Mine for Proteins Carrying A3FG1

Total serum proteins from advanced breast cancer and controls were subjected to 2D electrophoresis (pI 3-10) followed by Western blotting using KM93, an antibody against the sialyl Lewis x epitope. Three glycoprotein spots were identified in the patient's blot, which were not observed in the control (FIG. 13a). These spots were excised and subjected to N-gylcan release for glycan sequencing, followed by trypsin digestion for protein identification by LC-MS/MS. All three spots contained the A3FG3S3 structure (data not shown) and identified as; i) α1 antichymotrypsin, ii) α1 acid glycoprotein and iii) haptoglobin β-chain (Table 2).

TABLE 2
A3FG1 bearing proteins identified in advanced breast cancer serum.

| Gel | | Swiss-Prot | Protein | Mass | pl |
|---|---|---|---|---|---|
| spot | Protein name | entry | score | (calc.) | (calc.) |
| i | Alpha-1 antichymotrypsin | AACT_HUMAN | 1013.05 | 47650 | 5.33 |
| | precursor | | | | |
| | Kininogen precursor | KNG_HUMAN | 418.81 | 71945 | 6.34 |
| | Vitronectin precursor | VTNC_HUMAN | 132.78 | 54305 | 5.55 |
| | Corticosteroid-binding | CBG_HUMAN | 46.03 | 45140 | 5.64 |
| | globulin precursor | | | | |
| ii | Alpha-1 acid glycoprotein 2 | A1AH_HUMAN | 162.61 | 23602 | 5.03 |
| | precursor | | | | |
| | Alpha-1 acid glycoprotein 1 | A1AG_HUMAN | 148.75 | 23511 | 4.93 |
| | precursor | | | | |
| | Alpha-1-antichymotrypsin | AACT_HUMAN | 76.69 | 47650 | 5.33 |
| | precursor | | | | |
| iii | Complement C3 precursor | CO3_HUMAN | 575.72 | 107164 | 6.02 |
| | Haptoglobin precursor | HPT_HUMAN | 440.78 | 45205 | 6.13 |
| | Complement C4 precursor | CO4_HUMAN | 45.05 | 192771 | 6.66 |

A3FG1 from Individual Proteins Versus Whole Serum

Once we have established that these proteins contributed to the increase in A3FG1 seen in serum, we examined A3FG1 levels in them individually to determine if the level of glycans on each of them increased during advanced breast cancer. To determine this, we quantified A3FG1 from NP HPLC profiles of N-glycan released from 2D spots of α1 acid glycoprotein, α1 anti chymotrypsin and the most acidic spot of haptoglobinβ chain excised from 80 μg (−1.5 μl) of total serum protein of each of the three samples of an individual patient (Patient A). The A3FG1 levels measured from N-glycan pools of these proteins were plotted alongside the A3FG1 quantfied from whole serum as well as CA 15-3 (FIG. 13b).

The trends for A3FG1 of specific proteins were similar to that of A3FG1 from whole serum in the first two samples, but all the protein specific A3FG1 measurements increased in the third sample and this showed that these measurements are better indicators of metastasis than CA 15-3 and the glycan marker in whole serum. This result suggests that the evaluation of these A3FG1-protein glycoforms could serve as an alternative for early detection of advanced breast malignancy.

Discussion

The serum N-linked glycan analysis was analysed by a combination of HPLCs with computer aided data analysis and mass spectrometry (MS) (Royle L et al. (2008) Analytical Biochem, 376, 1-12) techniques, from nine advanced breast cancer patients and ten female controls. The N-glycan profiles from both groups were compared and significant changes identified. A longitudinal case study was carried out to evaluate the possible correlation of the glycan changes with disease progression compared with the current clinical marker, CA 15-3. Combining glycan analysis with proteomics allowed the identification of glycoproteins which contributed to the altered glycosylation observed in breast cancer serum.

By comparison with a standard serum glycome database and individual age-matched controls, breast cancer samples showed increased outer arm fucosylation, more specifically a tri-sialylated tri-antennary structure with an α1-3 linked fucose which forms the sialyl Lewis x epitope. Following a combination of sialidase and β-galactosidase digestion, its digestion product, a mono-galactosylated tri-antennary structure with an α1-3 linked fucose, was accurately quantified. Patients also had elevated levels of agalactosylated fucosylated bi-antennary glycan compared to controls.

Altered N-Glycosylation of Glycoproteins in Breast Cancer Serum

Alterations in the N-linked glycosylation in cancer as well as other diseases has gained a lot of research interest and has shown potential as disease markers and for immunotherapy of tumours. Using robust and highly sensitive technology, the N-glycans of total serum proteins from breast cancer were analysed in search of aberrant structure(s) that could distinguish between breast cancer patients and controls.

Increased levels of tri-sialylated tri-antennary structures with α1,3 fucose, which forms the epitope SLe^{x}, were identified in patients compared to controls (FIG. 9a). This data indicates increased branching in breast cancer serum. The addition of GlcNAc to the tri-mannosyl core of complex N-linked structures is mediated by the enzyme GnT-V, whose transcription has been shown to be stimulated by oncogenes, including her-2/neu. Synthesis of SLe^{x} is known to require sialylation to precede fucosylation of the internal GlcNAc residues by ST3Gal-IV and VI. These results suggest that there is increased activity of sialyltransferases (ST) in breast cancer, as reported previously by measurements of the respective levels in patient serum and tissue, both of which correlated with disease progression. The main fucosyltransferase involved in the synthesis of SLe^{x} is the FucT VI, whose gene expression correlates with SLe^{x} expression on the surface of breast cancer cells (Matsuura N. et al. 1998). SLe^{x} expression on MUC1 on breast cancer cell surface decreased in MARY-X, the human SCID model of inflammatory breast cancer, due to decreased level of α1,3 fucosyltransferase activity. This resulted in lack of binding to the surrounding endothelium, no electrostatic repulsion between cells and spheroid formation which also contributed to the overexpression of E-cadherin. All these effects were reversed by transfection with FucT-III cDNA.

The nm23-H1 suppressor gene has been reported to correlate inversely with SLe^{x} expression on breast cancer cells, influencing disease-free survival rates of patients. Recently, the mechanism was explained by Duan et al. who reported that nm23-H1 downregulates the genes and protein expression of GnT-V, ST and FucT resulting in reduced SLe^{x} expression and lower metastatic potential.

The levels of the SLe^{x} glycan marker, in the form of A3FG1, were quantified in all advanced breast cancer patients and controls (FIG. 11). The results indicate that breast cancer patients have on average a 3-fold increased level of SLe^{x} in the serum compared to controls. Also observed were increased SLe^{x} in the serum of advanced ovarian, lung, prostate cancer, as well as inflammatory conditions namely sepsis and pancreatitis. These results, taken together confirm that SLe^{x} present in the serum is not a marker for a specific malignancy or other disease condition in agreement with the conclusion that its expression level on cell surface also did not correlate with a specific disease.

However, this glycan marker could be a useful indicator of breast cancer progression and metastases in individual patients. In the case study of patient A, the level of A3FG1 was found to be better than CA 15-3 in indicating metastasis. There have been various reports that support the usefulness of serum SLe^{x} evaluation in breast cancer. Measurement of serum SLe^{x} was previously carried out by Kurebayashi et al. using a radioimmunoassay (RIA) kit Fhβ-Otsuka (Otsuka Assay Laboratory) with a cutoff value of value 38 U/ml (Kurebayashi J. et al. Jpn J Clin Oncol 2006; 36:150-3). In this study, SLe^{x} when used in combination with CA 15-3, increased the number of detected cases to 78.5%, compared to CA 15-3 on its own (61.5%) or the combination of CA 15-3 with CEA (72.3%). Similiarly, high serum SLe^{x}, predicts multilevel N2 stage and poor outcome of non-small cell lung cancer (NSCLC) and has been suggested useful as a staging marker in this case. Serum SLe^{x} also correlates with the soluble form of its ligand, E-selectin, in advanced and recurrent breast cancer.

In order to understand the rationale behind the increased serum levels of SLe^{x}, it was crucial to determine the proteins carrying this structure. The acute phase proteins, α1 acid glycoprotein (AGP), α1 antichymotrypsin (ACT) and haptoglobin β-chain (Hap) have all been previously reported to carry complex glycan structures with the SLe^{x} epitope. The SLe^{x} glycan was identified directly from these proteins from breast cancer serum separated by 2D electrophoresis followed by immunoblotting and glycan analysis (FIG. 13a). AGP is classified as a positive acute phase reactant and has 5 potential N-glycosylation sites, making it one of the most heavily glycosylated serum proteins. Alterations of AGP glycosylation is often observed together with two other acute phase proteins, α1-protease inhibitor and ACT.

AGP glycosylation, particularly the degree of branching and fucosylation, have been associated with various cancers and inflammatory diseases and act as putative markers such as in fibrosis. Duche et al. measured plasma AGP concentrations in breast, lung and ovary cancer patients and showed increased levels in all cancer groups compared to controls. The genetic variants of AGP appeared similar to that of controls, but expression levels were increased accordingly with its concentration (Duche, J. C., et al. Clin Biochem, 2000. 33(3): p. 197-202).

The biological role of AGP in diseases focuses mainly on the SLe^{x} structure that it carries. Its anti-inflammatory role involves high expression of SLe^{x} interfering with the selectin mediated endothelial-leukocyte adhesion when E-selectin expression is enhanced by pro-inflammatory cytokines. Similiarly in cancers, high concentrations of AGP carrying SLe^{x} results in a higher amount of binding to E-selectin on endothelial cells which competes with cell surface SLe^{x}. This supports the hypothesis that circulating SLe^{x} exerts a feedback inhibitory effect on the extravasation of cancer cells, resulting in a defense mechanism against metastasis. Low levels of serum glycolipid SLe^{x} in colon cancer was also found to correlate with higher recurrence and shorter disease-free interval. The concept of inhibiting the SLe^{x}-E-selectin interaction for therapeutics design was employed by Fukami et al. who showed that metastasis could be suppressed by using a Macrospelide B, which blocks SLe^{x} binding to E-selectin (Fukami, A., et al. Biochem Biophys Res Commun, 2002. 291(4): p. 1065-70).

Havenaar 1998 looked at AGP α1,3 fucosylation in pregnant women and found that there was a steady increase in branching and decrease in fucosylation (only up to week 25) which was similar to that observed in RA patients who went into remission during pregnancy, suggesting the influence of oestrogen on AGP glycosylation (Havenaar), probably by influencing the expression of cytokine genes which acts on the liver machinery. Brinkman-van der linden 1998 also showed the effect of oestrogen in reducing SLe^{x} expression, contrast to the acute inflammation (Cid MC 1994).

AGP and ACT have been shown to be synthesised by human breast epithelial cells, and interestingly, had increased levels in MCF-7 culture media. This suggests the possibility that both aberrant forms of AGP and ACT might come from the breast tumour and not solely from the liver, as generally understood. This is also strengthened by the fact that the breast cancer cells express the required glycosyltransferases to produce altered glycoforms of AGP and ACT. ACT, is also an estrogen-inducible gene, and its mRNA expression was shown to predict early tumour recurrence in invasive breast cancer patients.

### Example 3

### Ovarian Cancer

Materials and Methods

Serum Samples

Venous blood samples were obtained from a) healthy controls and patients undergoing treatment at St James's University Hospital in Leeds, UK and b) from healthy donors and melanoma patients participating in a research program of the Institute of Biochemistry, Bucharest, following ethical approval and obtaining informed consent. After allowing the blood to clot for 30-60 minutes, serum was obtained by centrifugation at 2,000 g for 10 minutes and stored at −80° C. until analysis.

a) For the initial pilot study, samples from 3 patients with advanced ovarian cancer were used (Patient A, stage IIIC serous and endometrioid carcinoma prior to surgery; Patients B and C, stage III serous carcinoma at the time of relapse with advanced disease; age range 60-72 years) and compared with a serum pool formed from five females of similar age). For the screening of serum proteins carrying glycosylation changes pooled control serum formed from eight females of similar age was compared to pooled serum formed from three females with benign gynaecological conditions (principally serous adenoma or cysts); malignant ovarian cancer (one serous and endometrioid carcinoma, one bilateral serous adenocarcinoma and one bilateral papillary adenocarcinoma); and metastatic ovarian cancer (two papillary serous adenocarcinoma, one serous carcinoma). For the main part of the study, samples from a further 90 controls and patients with ovarian cancer, other gynaecological cancers or benign gynaecological conditions were used (Table 5). Serum concentrations of CRP were analysed using an Advia 1650 analyser (Bayer, Newbury, UK) and CA125 using a Centaur analyser (Bayer). Reference ranges were <10 mg/L and <35 U/mL.

b) For the study, the following patients with malignant melanoma have been used to compare with 3 healthy controls (age range 35-52 years): 4 patients with malignant melanoma, pigmented, invasion Clark-3 to Clark-4 (3 non-ulcerated, 1 ulcerated, age range 30-58 years), 1 patient 6 months from surgery for a malignant melanoma tumour, posterior chest (36 years old), 1 patient with abdominal dysplastic nevus, 0.8 mm/0.2 mm diameter (47 years old) and 1 patient with hyperpigmented malignant melanoma tumours, located on anterior chest and underclavicula (52 years old). This patient had as previous tumour and underwent surgery and chemotherapy. Fibrinogen was determined as clottable protein using the method described by Swaim and Feders (Swaim W. R. and Feders, M. B. (1967) Clin Chem, 13, 1026-1028.). Reference ranges were 200-400 ng/ml.

Release and Purification of N-Glycans from Human Serum in Gel Block

N-glycans were released from glycoproteins in serum samples by in situ digestion with N-glycosidase F (PNGase F, Roche, Mannheim, Germany)

a) in SDS—PAGE gel bands as described earlier (Royle L. et al. (2006) Methods Mol Biol, 347, 125-143) or b) in-gel blocks as described by Royle et al. (Royle L et al. (2008) Analytical Biochem, 376, 1-12). Briefly, serum samples were reduced and alkylated, then set into SDS-gel blocks, washed and N-glycan released by PNGase F.

Fluorescent Labeling of the Reducing Terminus of N-Glycans

Glycans were fluorescently labelled with 2-aminobenzamide (2AB) by reductive amination (Bigge et al. 1995) (LudgerTag 2-AB labeling kit Ludger Ltd., Abingdon, UK).

Exoglycosidase Digestion of 2AB Labeled N-Linked Glycans

All enzymes were purchased from Glyko (Novato, Calif.) or New England Biolabs (Hitchin, Herts, UK). The 2AB-labelled glycans were digested in a volume of 10 μl for 18 h at 37° C. in 50 mM sodium acetate buffer, pH 5.5 (except in the case of JBM where the buffer was 100 mM sodium acetate, 2 mM Zn²⁺, pH 5.0), using arrays of the following enzymes:

ABS—Arthrobacter ureafaciens sialidase (EC 3.2.1.18), 1 U/ml; NAN1-Streptococcus pneumoniae sialidase (EC 3.2.1.18), 1 U/ml; BTG—bovine testes β-galactosidase (EC 3.2.1.23), 1 U/ml; SPG—Streptococcus pneumoniae β-galactosidase (EC 3.2.1.23), 0.1 U/ml; BKF—bovine kidney alpha-fucosidase (EC 3.2.1.51), 1 U/ml; GUH-β-N-acetylglucosaminidase cloned from Streptococcus pneumonia, expressed in E. coli (EC 3.2.1.30), 4 U/ml; JBM—jack bean α-mannosidase (EC 3.2.1.24), 50 U/ml; AMF—Almond meal alpha-fucosidase (EC 3.2.1.111), 3 mU/ml, XMF—Xanthomonus sp. alpha-fucosidase (EC 3.2.1.51.), 0.1 U/ml. After incubation, enzymes were removed by filtration through a protein binding EZ filters (Millipore Corporation, Beford, Mass., USA) (Royle et al. 2006), the N-glycans were then analyzed by NP-HPLC and WAX-HPLC.

HPLC

NP-HPLC was performed using a TSK-Gel Amide-80 4.6×250 mm column (Anachem, Luton, UK) on a 2695 Alliance separations module (Waters, Milford, Mass.) equipped with a Waters temperature control module and a Waters 2475 fluorescence detector. Solvent A was 50 mM formic acid adjusted to pH 4.4 with ammonia solution. Solvent B was acetonitrile. The column temperature was set to 30° C. Gradient conditions were a linear gradient of 26-52% A, over 104 min at a flow rate of 0.4 ml/min. Samples were injected in 74% acetonitrile (Royle L et al. (2008) Analytical Biochem, 376, 1-12). Fluorescence was measured at 420 nm with excitation at 330 nm. The system was calibrated using an external standard of hydrolyzed and 2AB-labeled glucose oligomers to create a dextran ladder, as described previously (Royle L. et al. (2006) Methods Mol Biol, 347, 125-143).

WAXHPLC was performed using a Vydac 301VHP575 7.5×50 mm column (Anachem, Luton, Bedfordshire, UK) as described (Royle L. et al. (2006)

Methods Mol Biol, 347, 125-143). Briefly, solvent A was 0.5 M ammonium formate pH 9. Solvent B was 10% (v/v) methanol in water. Gradient conditions were a linear gradient of 0-5% A over 12 min at a flow rate of 1 ml/min, followed by 5-21% A over 13 min, then 21-50% A over 25 min, 80-100% A over 5 min, then 5 min at 100% A. Samples were injected in water.

MALDI-TOF MS

Positive ion MALDI-TOF mass spectra were recorded with a Micromass TofSpec 2E reflectron-TOF mass spectrometer (Micromass, Manchester, United Kingdom) fitted with delayed extraction and a nitrogen laser (337 nm). The acceleration voltage was 20 kV; the pulse voltage was 3200 V; the delay for the delayed extraction ion source was 500 ns. Samples were prepared by adding 0.5 ml of an aqueous solution of the sample to the matrix solution (0.3 ml of a saturated solution of 2,5-dihydroxybenzoic acid in acetonitrile) on the stainless steel target plate and allowing it to dry at room temperature. The sample/matrix mixture was then recrystallized from ethanol (Harvey, D. J. (1993) Rapid Commun Mass Spectrom, 7, 614-619).

Negative Ion Electrospray Ionisation Mass Spectrometry ESI-MS and ESI MS/MS

Nano-electrospray mass spectrometry was performed with a Waters-Micromass quadrupole-time-of-flight (Q-T of) Ultima Global instrument.

Samples in 1:1 (v:v) methanol:water containing 0.5 mM ammonium phosphate were infused through Proxeon (Proxeon Biosystems, Odense, Denmark) nanospray capillaries. The ion source conditions were: temperature, 120° C.; nitrogen flow 50 L/hr; infusion needle potential, 1.2 kV; cone voltage 100 V; RF-1 voltage 150 V. Spectra (2 sec scans) were acquired with a digitization rate of 4 GHz and accumulated until a satisfactory signal:noise ratio had been obtained. For MS/MS data acquisition, the parent ion was selected at low resolution (about 4 m/z mass window) to allow transmission of isotope peaks and fragmented with argon. The voltage on the collision cell was adjusted with mass and charge to give an even distribution of fragment ions across the mass scale. Typical values were 80-120 V.

Purification of Serum IgG

Serum (5 μl) was diluted 100-fold with 0.1 M Tris, 1 M NaCl, 1 mM EDTA, pH 7.5 and applied to a Protein G column (Pharmacia Biotech, Uppsala, Sweden). The column was equilibrated and washed with 15 ml of 0.1 M Tris, 1 M NaCl, 1 mM EDTA, pH 7.5 and the IgG was eluted with 0.1 M glycine-HCl, pH 2.7 into 1.5 ml tubes containing 100 μl 0.1 M Tris 1 M NaCl 1 mM EDTA buffer (pH 7.5). The fractions containing IgG were pooled and dialyzed against 1×PBS overnight at 4° C. using a dialysis membrane (Medicell International Ltd., London, UK). The dialysed IgG was concentrated by adding 10 μl resin (Strata clean resin, Stratagene, La Jolla, Calif., USA) and left at room temperature for 1 hour at slow rotation for binding. After centrifugation at 1000 g the supernatant was removed to leave about 10 μl of pellet in the bottom of the tube, this was reduced and alkylated and transferred to SDS-PAGE gel. Following electrophoresis the pure IgG heavy chain band was cut out from the gel for glycan analysis.

Electrophoresis

Electrophoresis in 4-12% Bis-Tris SDS PAGE mini-gels (Invitrogen, Carlsbad, Calif., USA) was performed at room temperature according to the method of Laemmli (Laemmli 1970). The gels were Coomassie stained. All samples were reduced with 5% 2-mercaptoethanol before analysis. Approximately 40 μg of proteins from sera was loaded per lane.

2-Dimensional Electrophoresis (2-DE)

Eighty micrograms of the human serum were dissolved in 120 μl of sample buffer (5 M urea, 2 M thiourea, 2 mM tributyl-phosphine, 65 mM DTT, 4% CHAPS, 4% v/w NDSB-256, trace of bromophenol blue) and subjected to 2-DE. Ampholytes were added to the sample at 0.9% Servalyte 3-10, 0.45% Servalyte 2-4 and 9-11. Immobilized pH gradient gels (Immobiline DryStrip 3-10 NL, 7 cm) were rehydrated in the sample and IEF was carried out according to method described by Sanchez (Sanchez, J. C. et al. (1997) Electrophoresis, 18, 324-327) at 17° C. but with modified voltages and times as following: first minute 200 V, 3 mA, 5 W, then 3500 V, 3 hours and 30 minutes, 10 mA, 5 W. Following focusing, the IPG strips were immediately equilibrated for 15 minutes in 4 M urea, 2 M thiourea, 2% (w/v) DTT, 30% glycerol, 50 mM Tris, pH 6.8, 2% SDS, trace of bromophenol blue. Proteins were separated in the second dimension at 125 V for 2 hours, RT, on 4-12% Bis-Tris gradient gels (Invitrogen, Carlsbad, Calif., USA). Following electrophoresis, the gels were fixed in 40% (v/v) ethanol: 10% (v/v) acetic acid and stained with the fluorescent dye OGT 1238 (Oxford Glycosciences, Abingdon, UK) according to the method previously described (Hassner A. (1984) Synthesis. J Org Chem, 49, 2546-2551). Monochrome fluorescence images were obtained by scanning gels with an Apollo H linear fluorescent scanner (Oxford Glycosciences).

Glycan Analysis of 2-DE Gel Spots

Glycans were released and extracted from the 1 mm³ of gel excised for MS analysis. The procedure used was the in gel block method for human serum, with modifications. The gel pieces were frozen for >2 hours and then washed for 15 minutes with shaking with alternating 1 ml acetonitrile and 1 ml 20 mM NaHCO₃ for 3 washes. After each step the washings were removed under vacuum. The glycoproteins were not reduced and alkylated before loading on the gel therefore reduction and alkylation were carried out in situ: the gel pieces were incubated at 37° C. for 30 minutes with 20 μl 0.5 M DTT plus 180 μl 20 mM NaHCO₃ then 20 μl 100 mM IAA were added and incubation continued for a further 30 minutes at RT. The procedure then followed the in-gel block method starting with 5 alternating washes with acetonitrile and 20 mM NaHCO₃. Sufficient PNGaseF was added to cover the gel pieces. Released glycans were eluted with 3 washes with 200 μl water and another 3 alternating washes with 200 μl acetonitrile and 200 μl water, each wash for 30 minutes, formic acid treated and labelled with the fluorophore 2AB as described earlier (Bigge, J. C. et al. (1995) Anal Biochem, 230, 229-238). Sufficient glycans were produced by these procedures for up to 10 NP-HPLC chromatograms, including digestions. The proteins which remained in the gel spots were identified by mass spectrometry.

Identification of Proteins in Gel Spots from 2-DE (See Table 7) by Mass Spectrometric Analysis

Mass spectrometric analysis was carried out using a Q-TOF 1 (Micromass, Manchester, UK) coupled to a CapLC (Waters, Milford, Mass., USA). Tryptic peptides were concentrated and desalted on a 300 μm id/5 mm C18 precolumn and resolved on a 75 μm id/25 cm C18 PepMap analytical column (LC packings, San Francisco, Calif., USA). Peptides were eluted to the mass spectrometer using a 45 min 5-95% acetonitrile gradient containing 0.1% formic acid at a flow rate of 200 nl/min. Spectra were acquired in positive mode with a cone voltage of 40 V and a capillary voltage of 3300 V. The MS to MS/MS switching was controlled in an automatic data dependent fashion with a 1 second survey scan followed by three 1 second MS/MS scans of the most intense ions. Precursor ions selected for MS/MS were excluded from further fragmentation for 2 minutes. Spectra were processed using ProteinLynx Global server 2.1.5 and searched against the SWISS-PROT and NCBI databases using the MASCOT search engine (Matrix science, London, UK). Searches were restricted to the human taxonomy allowing carbamidomethyl cysteine as a fixed modification and oxidized methionine as a potential variable modification. Data was searched allowing 0.5 Da error on all spectra and up to two missed tryptic cleavage sites to accommodate calibration drift and incomplete digestion, all data was checked for consistent error distribution.

Statistical Analysis

Non-parametric statistical tests were used with Kruskal Wallis test for comparison of all groups for SLe^{x} levels and subsequent Mann Whitney tests for comparison of individual groups. Correlation analysis was carried out using two-tailed Spearman test. In all cases a P<0.05 was taken as the cut-off level for significance.

Results

Identification of N-Glycosylation Changes in Serum Ovarian Cancer Patients

The N-glycans were identified using quantitative NPHPLC and exoglycosidase digestion with structural assignments made by using database matching (GlycoBase; URL—http://glycobase.ucd.ie/cgi-bin/public/glycobase.cgi) combined with matrix-assisted laser desorption/ionization time-of-flight (MALDI-TOF) and negative ion nanoelectrospray mass spectrometric analysis, as described earlier (Harvey, D. J. (2005a) J Am Soc Mass Spectrom, 16, 622-630, Harvey, D. J. (2005b) J Am Soc Mass Spectrom, 16, 631-646, Harvey, D. J. (2005c) J Am Soc Mass Spectrom, 16, 647-659, Royle L et al. (2008) Analytical Biochem, 376, 1-12). The N-linked glycosylation changes in 3 ovarian cancer patients were analyzed in a preliminary study to identify specific glycan structures, the levels of which were altered in the patient samples. The results from these sera were compared to those from a healthy control pool (5 normal sex- and age-matched serum samples).

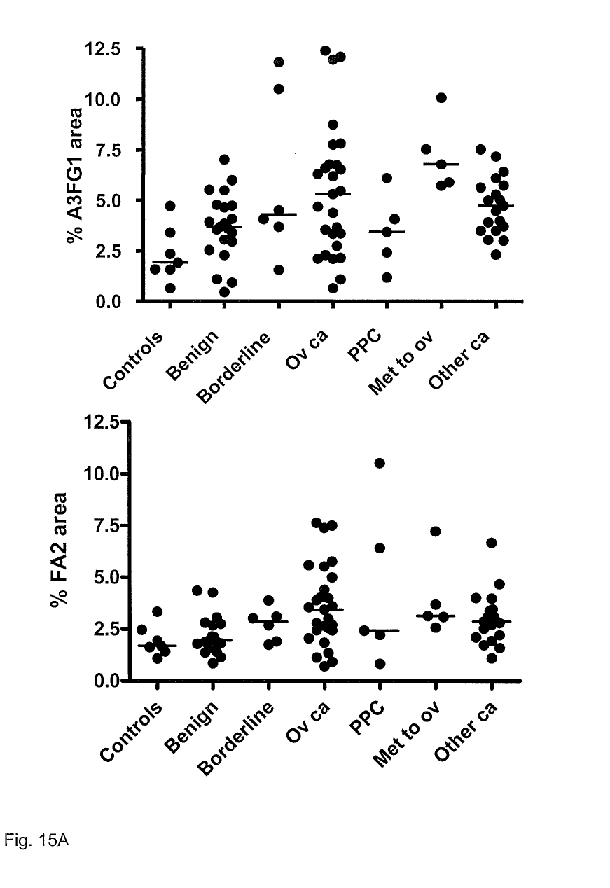

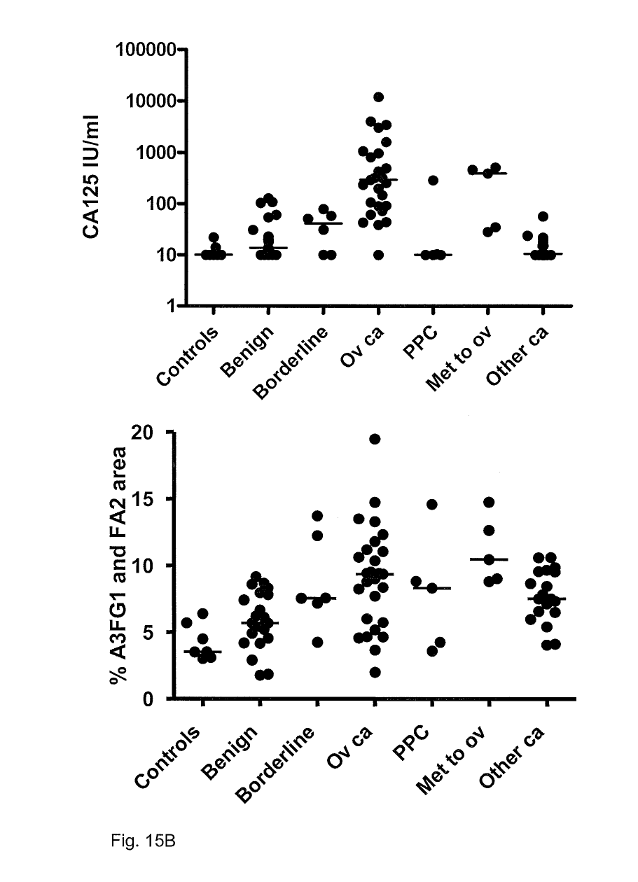

Whole serum glycans from 3 patients were fractionated on WAXHPLC according to charge and each fraction was subsequently analyzed by NPHPLC, represented by the profiles from a stage III ovarian cancer patient (B) and the control sample (FIG. 14). The relative amounts of sialylated glycans were calculated from WAXHPLC (Table 3).

TABLE 3
Summary of glycans identified as altered
in ovarian cancer patients.

| | Relative % Area of charged Glycans | | | |
|---|---|---|---|---|
| sample | monosialylated | disialylated | trisialylated | tetrasialylated |
| Patient A | 13.0 | 58.3 | 25.1 | 3.5 |
| Patient B | 13.5 | 56.2 | 23.9 | 6.4 |
| Patient C | 14.8 | 56.9 | 24.0 | 4.3 |
| Patient | 13.8 ± 0.8 | 57.1 ± 0.9 | 24.3 ± 0.6 | 4.7 ± 1.2 |
| average | | | | |
| Control | 24.6 | 58.1 | 14.3 | 3.1 |

From these data, the levels of monosialylated glycans from the patient samples were about half that of the control pool whilst there were increased (approximately double) levels of the tri and tetrasialylated glycans. There was no significant change in the relative amounts of disialylated glycans. Glycan structures in the fractions were confirmed using exoglycosidase digestions, NPHPLC and MALDI MS. Percentage areas of each glycan from WAX fractions and in whole serum are shown in Table 4 which summarises the glycans identified in NPHPLC chromatograms of the WAX fractions and the levels of them.

TABLE 4
Relative % areas of charged glycans from WAXHPLC

| fraction | neutral | |
|---|---|---|
| ¹peak ID | 1 | 2 |
| ²structure | 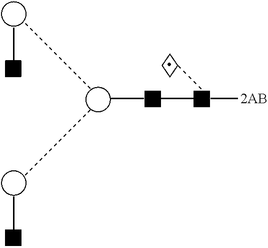 | 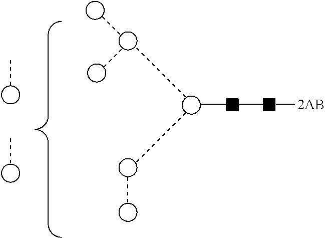 |
| abbreviation | FA2 | M8 |
| ³MS | Hex3HexNAc4 | Hex8HexNAc2 |
| | Fuc1 | |
| GU | 5.82 | 8.89 |
| patient A | 27.7 | 3.2 |
| patient B | 20.9 | 3.8 |
| patient C | 32.4 | 4.1 |
| patient average | 27.0 ± 4.7 | 3.7 ± 0.4 |
| control | 10.8 | 5.7 |
| glycoprotein | IgG | |
| fraction | neutral | |
| ¹peak ID | 3 | 4 |
| ²structure | 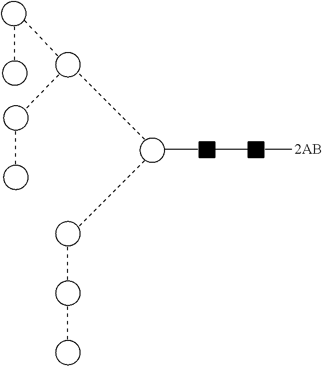 | 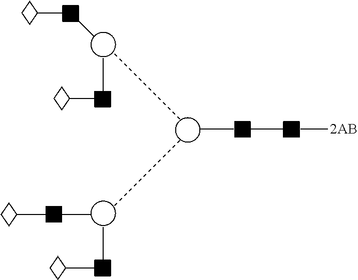 |
| abbreviation | M9 | A4G4 |
| ³MS | Hex9HexNAc2 | Hex7HexNAc6 |
| GU | 9.49 | |
| patient A | 9.7 | |
| patient B | 7.0 | |
| patient C | 8.4 | |
| patient average | 8.4 ± 1.1 | |
| control | 6.1 | |
| glycoprotein | | |
| fraction | monosialylated | |
| ¹peak ID | 5 | 6 |
| ²structure | 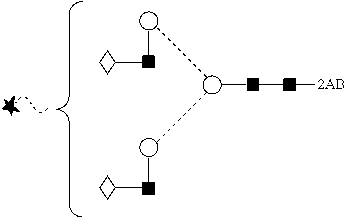 | 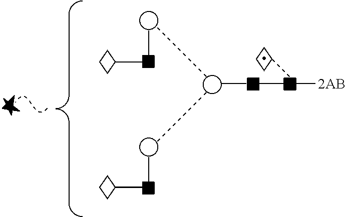 |
| abbreviation | A2G2S1 | FA2G2S1 |
| ³MS | Hex5HexNAc4 | Hex5HexNAc4 |
| | NeuNAc1 | Fuc1NeuNAc1 |
| GU | 8.08 | 8.46 |
| patient A | 39.6 | 10.5 |
| patient B | 48.5 | 15.0 |
| patient C | 40.2 | 16.0 |
| patient average | 42.8 ± 4.1 | 13.8 ± 2.4 |
| control | 34.4 | 18.0 |
| glycoprotein | | |
| fraction | monosialylated | disialylated |
| ¹peak ID | 7 | 8 |
| ²structure | 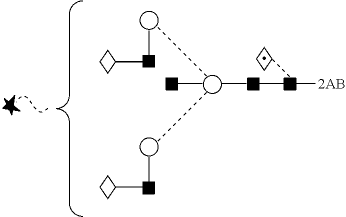 | 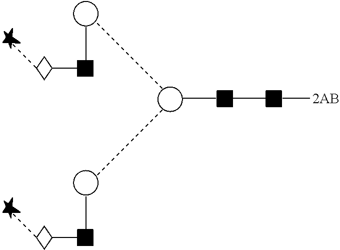 |
| abbreviation | FA2BG2S1 | ⁴A2G2S(6,6)2 |
| ³MS | Hex5HexNAc5 | Hex5HexNAc4 |
| | Fuc1NeuNAc1 | NeuNAc2 |
| GU | 8.63 | 8.86 |
| patient A | 8.2 | 64.0 |
| patient B | 8.1 | 61.5 |
| patient C | 11.0 | 61.1 |
| patient average | 9.1 ± 1.4 | 62.2 ± 1.3 |
| control | 16.4 | 59.0 |
| glycoprotein | | |
| fraction | disialylated | |
| ¹peak ID | 9 | 10 |
| ²structure | 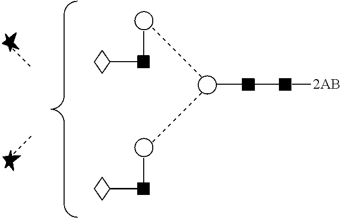 | 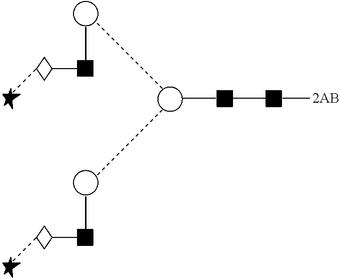 |
| abbreviation | ⁴A2G2S(3,6)2 | ⁴A2G2S(3,3)2 |
| ³MS | Hex5HexNAc4 | Hex5HexNAc4 |
| | NeuNAc2 | NeuNAc2 |
| GU | 8.86 | 8.86 |
| patient A | 33.8 | 2.2 |
| patient B | 35.7 | 2.8 |
| patient C | 36.0 | 2.9 |
| patient average | 35.2 ± 1.0 | 2.6 ± 0.3 |
| control | 37.6 | 3.4 |
| glycoprotein | | |
| fraction | trisialylated | |
| ¹peak ID | 11 | 12 |
| ²structure | 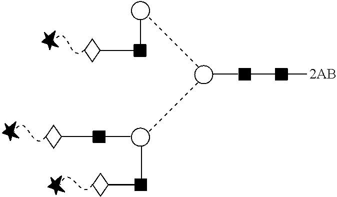 | 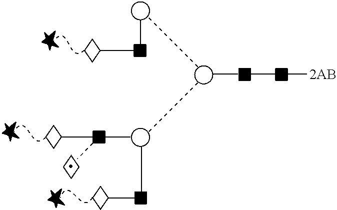 |
| abbreviation | A3G3S3 | ⁶A3F1G3S3 |
| ³MS | Hex6HexNAc5 | Hex6HexNAc5 |
| | NeuNAc3 | Fuc1NeuNAc3 |
| GU | 10.00 | 10.50 |
| patient A | 12.3 | 65.3 |
| patient B | 24.8 | 58.3 |
| patient C | 33.7 | 57.6 |
| patient average | 23.6 ± 8.8 | 60.4 ± 3.5 |
| control | 39.6 | 46.1 |
| glycoprotein | ⁵HP, AGP, ACH | |
| fraction | whole serum | |
| ¹peak ID | 1 | 12 |
| ²structure | 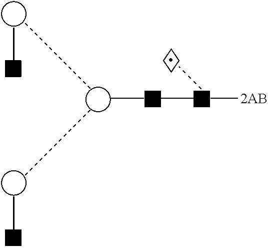 | 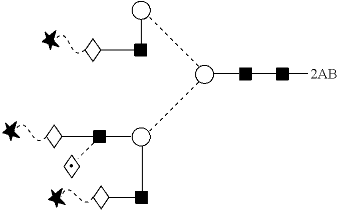 |
| abbreviation | FA2 | ⁶A3F1G3S3 |
| ³MS | Hex3HexNAc4 | Hex6HexNAc5 |
| | Fuc1 | Fuc1NeuNAc3 |
| GU | 5.82 | 10.50 |
| patient A | 2.9 | 17.7 |
| patient B | 2.7 | 13.8 |
| patient C | 4.5 | 12.9 |
| patient average | 3.4 ± 0.8 | 14.8 ± 2.1 |
| control | 1.9 | 6.5 |
| glycoprotein | IgG | ⁵HP, AGP, ACH |

¹Peak ID relates to FIG. 1
²Structure abbreviations: all N-glycans have 2 core GlcNAcs; F at the start of the abbreviation indicates a core fucose α1-6 to inner GlcNAc; Man (x), number (x) of mannose on core GlcNAcs; A(x), number (x) of antenna (GlcNAc) on trimannosyl core; B, bisecting GlcNAc linked β1-4 to β1-3 mannose; F(x), number (x) of fucose linked α1-3 to antenna GlcNAc, G(x), number (x) of galactose on antenna; S(x), number of sialic acids on antenna. All structures were confirmed by exoglycosidase sequencing and also by MALDI MS from composition as [M + Na]+ ions, all masses within 0.2 Da of calculated.
Symbol representation of glycans in as follows: GlcNAc, filled square; mannose, open circle; galactose, open diamond; fucose, diamond with a dot inside; beta linkage, solid line; alpha linkage, dotted line; 1-4 linkage, horizontal line; 1-3 linkage, (/); 1-2 linkage, vertical line; and 1-6 linkage, (\).
³ID by MALDI and ESI MS/MS fragmentation
⁴Identified by digestion by α2-3 specific sialidase NAN1
⁵HP = haptoglobin β-chain, AGP = α1-acid glycoprotein and ACH = α1-antichymotrypsin
⁶Contains a SLe^{x} epitope

In the neutral fractions of the serum N-linked glycans: the core fucosylated biantennary glycan (FA2) is increased from 10.8% to 27.0(±4.7) % in patients; Man₈GlcNAc₂ (M8) is decreased from 5.7% to 3.7±(0.4) % in cancer; whereas the peak containing both Man₉GlcNAc₂ (M9) and the tetragalactosylated tetra-antennary structure (A4G4) is increased from 6.1% to 8.4(±1.1) %.

In the mono-sialylated N-linked glycan fractions, there is a decrease in fucosylation in the cancer samples. The core fucosylated digalactosylated monosialylated structures with and without bisects (B), FA2G2S1 and FA2BG2S1, are reduced from 18.0% to 13.8(±2.4) % (and 16.4% to 9.1(±1.4) % respectively), whilst the digalactosylated monosialylated structures (A2G2S1) are increased from 34.4% to 42.8(±4.1) % in the stage III.

In the di-sialylated fractions the amount of α2,3 sialic acid levels were only slightly lower compared to α2,6 sialic acid levels in stage III ovarian cancer than in the control. In Table 4 it is shown that A2G2S(6,6)₂ is increased from 59.0% to 62.2(±1.3) % but A2G2S(3,6)₂ is decreased from 37.6% to 35.2(±1.0) % and A2G2S(3,3)₂ is decreased from 3.4% to 2.6(±0.3) %. These structures were confirmed by NAN1 sialidase which digests only α2,3 links.

The tri-sialylated fractions showed increased outer arm fucosylation in cancer. A SLe^{x}-containing tri-antennary glycan (A3F1G3S3) is increased from 46.1% to 60.4(±3.5) % whereas the tri-sialylated non-fucosylated glycan (A3G3S3) is decreased from 39.6% to 23.6(±8.8) % in stage III ovarian cancer.

Overall the most striking differences between the cancer serum glycans and those from healthy controls, which are also clearly observed in the unfractionated whole serum glycan pool, are the doubling in the levels of A3FG1 (increase from 6.5% to 14.8(±2.1) %) and FA2 (increase from 1.9% to 3.4(±0.8) %).

A more extensive study into the levels of SLe^{x}, FA2 and CA125 was carried out on 90 serum samples from healthy controls, patients with benign gynaecological conditions, borderline ovarian tumours, ovarian cancer, primary peritoneal carcinomatosis, endometrial cancer metastasised to ovary and other gynaecological cancers (FIG. 15). The released glycans were digested with sialidase and β1-4 galactosidase to give the structure A3F1 G1. This digestion segregates the SLe^{x} containing structures from any others which are digested to lower GU value peaks, leaving a clearly separated peak for integration to give accurate percentage of total glycans.

Analysis of SLe^{x} only clearly shows significantly elevated levels in patients with ovarian cancer compared with healthy controls (p<0.01) although the number of control samples is small (n=7) and covers a slightly younger age range (see Table 5). However the difference between patients with other cancers or cancers which had metastasised to the ovary compared with controls was more marked (p<0.002). Additionally the patients with benign gynaecological conditions also showed levels which overlapped considerably and were not significantly different from those of the cancer patients. This contrasts markedly with CA125 results, which show much better specificity for the ovarian cancer group.

TABLE 5
Details of the female patient samples used in the
main part of the study for determination of SLe^{x}.

| | | Median age |
|---|---|---|
| | Number | (range) |
| Healthy controls | 7 | 55 (43-61) |
| Benign gynaecological conditions (principally | 21 | 44 (36-74) |
| endometriosis or cysts) | | |
| Borderline ovarian tumours | 6 | 67 (37-80) |
| Ovarian cancer (21 at presentation, 6 at | 27 | 58 (39-84 |
| relapse with advanced disease; 16 serous, | | |
| 11 = mucinous, endometrioid or clear cell; all | | |
| FIGO stages) | | |
| Primary peritoneal carcinomatosis | 5 | 67 (56-70) |
| Endometrial cancer metastasised to ovary (4 | 5 | 72 (43-90) |
| carcinoma, 1 sarcoma) | | |
| Other gynaecological cancers (16 | 19 | 67 (53-81) |
| endometrial carcinoma, 2 cervix, 1 fallopian | | |
| tube) | | |

Analysis of FA2 clearly shows significantly elevated levels in patients with ovarian cancer compared with healthy controls (p<0.022) and with benign gynaecological conditions (p<0.0054). The difference between patients with ovarian cancer and other gynaecological cancers was not significant. Analysis of FA2 combined with SLe^{x} clearly shows even more significantly elevated levels in patients with ovarian cancer compared with healthy controls (p<0.0016) and compared with benign gynaecological conditions (p<0.0016). However, the difference between patients with ovarian cancer and other gynaecological cancers was not significant. This suggests that combination of these two markers would improve the diagnosis of ovarian cancer.

The possibility that the changes in SLe^{x} reflect underlying inflammatory changes was examined by comparison with C-reactive protein (CRP) concentrations for all samples (unpublished data). A positive correlation was found (p<0.0023; r=0.32; Cl=0.12-0.5) but it was apparent that several patients showed marked acute-phase response but not elevated SLe^{x} levels and the converse was also true. This was particularly apparent for the patients in the “other cancer” group where only 5 patients out of 19 had CRP >10 mg/l. Correlation between CRP and CA125 was more positive (p<0.0001; r=0.41; Cl=0.22-0.57) then between CRP and SLe^{x}. Correlation between CRP and FA2 was not significant.

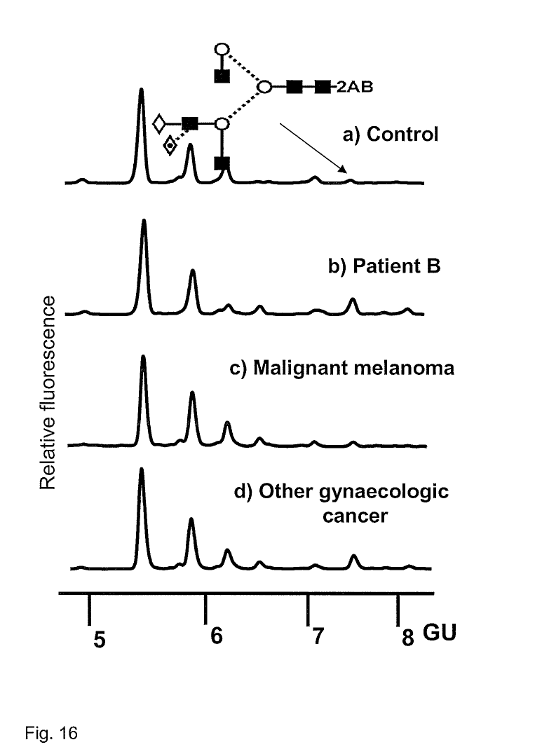

Interestingly, no change was identified in glycosylation of serum glycans in malignant melanoma samples compared to benign samples and control, where inflammation is not involved (FIG. 16). For all patients, the fibrinogen level was determined and the concentrations varied between 280 and 370 ng/ml. Normal values for this protein which increases in inflammation are 200-400 ng/ml. This confirms that these melanoma patients have a low level of inflammatory processes.

Identification of Glycoproteins Occupied by the Target Glycans

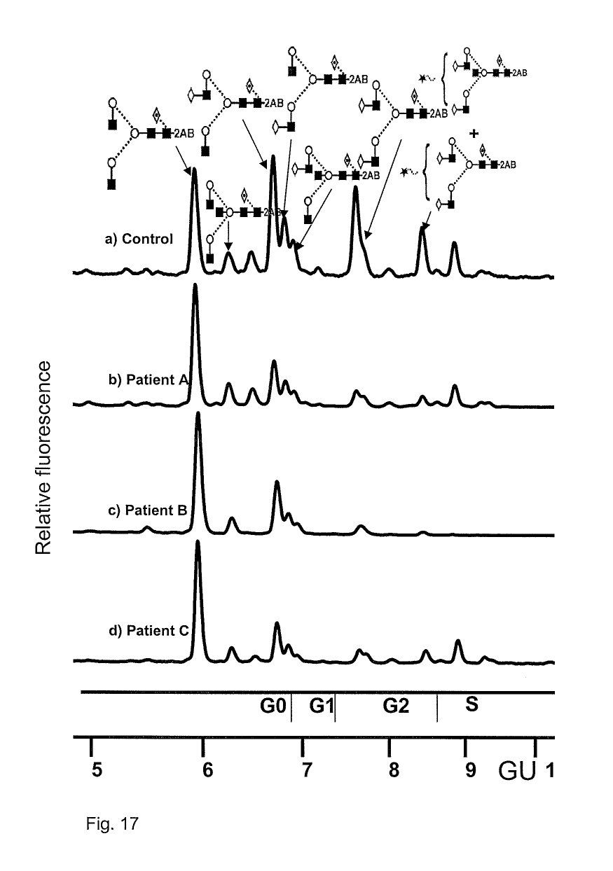

Having identified specific changes in glycan structures from whole serum glycoproteins, the next aim was to carry out some initial studies to identify which individual glycoproteins carried these glycans. A doubling in the level of FA2 glycan was found: this structure has previously been shown to be on immunoglobulin G (IgG). IgG was therefore isolated by affinity chromatography on a Protein G column and analysed the N-linked glycans from the heavy chain (FIG. 17 and Table β). IgG containing agalactosylated structures (G0) (mostly represented by FA2) were doubled (increased from 27.1% to 53.2(±3.3) %); monogalactosylated (G1) decreased (from 33.2% to 27.1(±5.3) %); digalactosylated (G2) structures decreased (from 22.3% to 8.5(±1.9) %); the overall sialylation decreased (from 17.5% to 11.2(±6.6) %) (Table β). All structures were confirmed by exoglycosidase digestions (Parekh, R. B. et al. (1985) Nature, 316, 452-457).

TABLE 6
Glycans from IgG heavy chain isolated by SDS-PAGE

| sample | G0 | G1 | G2 | S |
|---|---|---|---|---|
| Patient A | 50.5 | 25.0 | 9.7 | 14.9 |
| Patient B | 57.8 | 34.4 | 5.8 | 2.0 |
| Patient C | 51.4 | 21.8 | 10.0 | 16.8 |
| Patient average | 53.2 ± 3.3 | 27.1 ± 5.3 | 8.5 ± 1.9 | 11.2 ± 6.6 |
| Control | 27.1 | 33.2 | 22.3 | 17.5 |

NPHPLC percentage areas of neutral glycans (G0 = no galactose; G1 = 1 galactose; G2 = 2 galactoses) and sialylated glycans.

Haptoglobin β-chain has previously been shown to be aberrantly glycosylated in cancer. The serum proteome was examined to see if these and other glycoproteins showed glycosylation changes. 2D SDS-PAGE was employed to separate the ovarian cancer serum proteins, and then these protein spots were cut out and screened for possible altered glycosylation by glycan analysis of each individual spot.

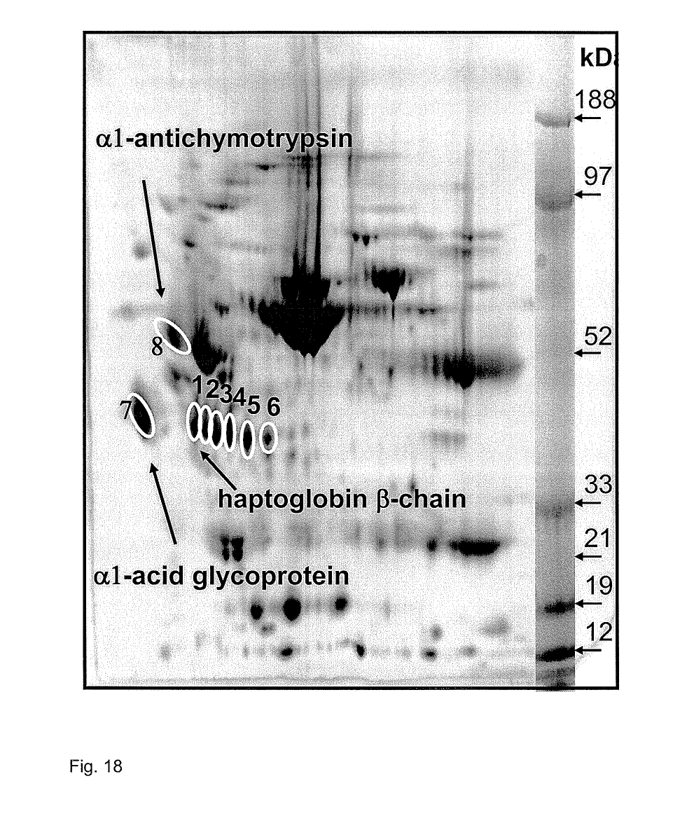

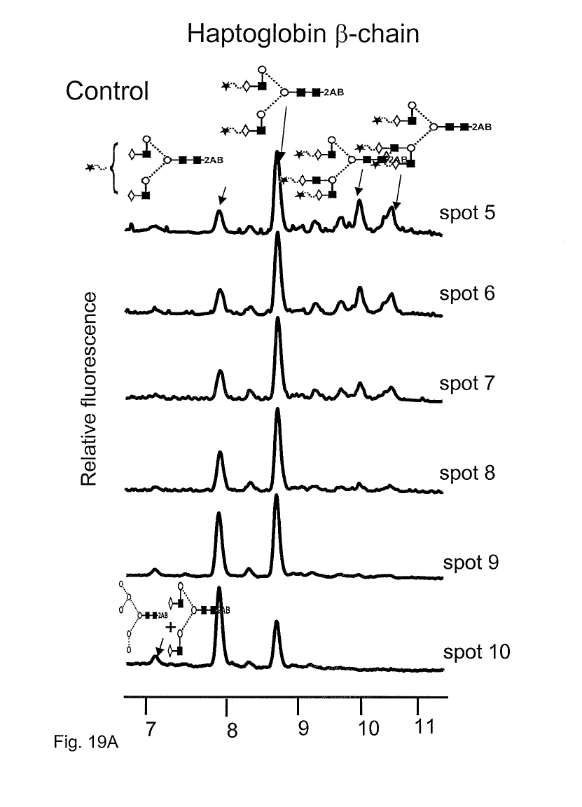

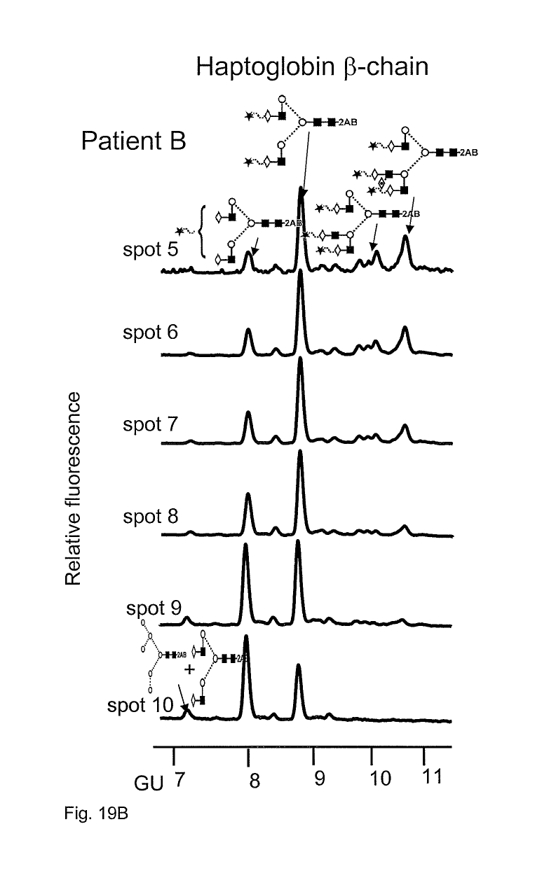

FIG. 18 shows 2D electrophoresis of total serum from a stage III ovarian cancer patient (B). N-glycans were released from these individual spots which were identified using mass spectrometric analysis (Table 7) to be haptoglobin β-chain glycoforms (He Z. et al. (2006) Biochem Biophys Res Commun, 343, 496-503), α1-acid glycoprotein and α1-antichymotrypsin.

TABLE 7
Identification of protein spots from 2-DE (shown in FIG. 18)
by nanospray-quadrupole time-of-flight-MS/MS of tryptic peptides
followed by MASCOT search of SWISS-PROT data base.

| gel | | accession | protein | protein | no. of |
|---|---|---|---|---|---|
| spot | ¹identification | number | covered (%) | score | peptides matched |
| 1 | Complement C3 | P01024 | 13.0 ± 0.5 | 729.5 ± 170.7 | 17 ± 2 |
| 1 | Haptoglobin β-chain | P00738 | 29.2 ± 0.6 | 421.7 ± 44.0 | 5 ± 4 |
| 1 | Zinc-α2-glycoprotein | P25311 | 12.7 ± 5.3 | 88.6 ± 2.7 | 3 ± 1 |
| 1 | Complement C4-A | P0C0L4 | 3.7 | 48.7 | 2 |
| 1 | Serum paraoxogenase/ | P27169 | 4.2 | 46.7 | 1 |
| | arylesterase 1 | | | | |
| 2 | Haptoglobin β-chain | P00738 | 36.3 ± 1.1 | 532.2 ± 78.2 | 12 ± 2 |
| 2 | Complement C3 | P01024 | 1.7 ± 0.5 | 88.3 ± 37.7 | 2 ± 1 |
| 2 | Serum paraoxogenase/ | P27169 | 4.2 | 71.6 ± 0.5 | 1 |
| | arylesterase 1 | | | | |
| 2 | Zinc-α2-glycoprotein | P25311 | 3.4 | 55.8 | 1 |
| 3 | Haptoglobin β-chain | P00738 | 28.0 ± 2.6 | 501.6 ± 4.5 | 11 ± 1 |
| 3 | Serum paraoxogenase/ | P27169 | 4.2 | 59.9 | 1 |
| | arylesterase 1 | | | | |
| 4 | Haptoglobin β-chain | P00738 | 37.0 ± 1.7 | 608.7 ± 40.8 | 14 |
| 4 | Serum paraoxogenase/ | P27169 | 4.2 | 63.9 | 1 |
| | arylesterase 1 | | | | |
| 5 | Haptoglobin β-chain | P00738 | 28.9 ± 0.4 | 547.0 ± 111.1 | 14 ± 2 |
| 6 | Haptoglobin β-chain | P00738 | 28.7 ± 3.8 | 458.4 ± 93.4 | 10 ± 2 |
| 7 | α1-acid glycoprotein | P02763 | 9.0 ± 4.0 | 87.9 ± 22.7 | 3 ± 1 |
| 8 | α1-antichymotrypsin | P01009 | 29.4 ± 13.2 | 571.2 ± 274.5 | 11 ± 5 |
| 8 | α1-antitrypsin | P01011 | 16.6 ± 9.5 | 134.7 ± 78.9 | 4 ± 2 |
| 8 | Kininogen-1 | P01042 | 6.5 ± 3.6 | 121.5 ± 76.6 | 3 ± 2 |

¹only glycoproteins identified in spots listed (unglycosylated proteins not listed)

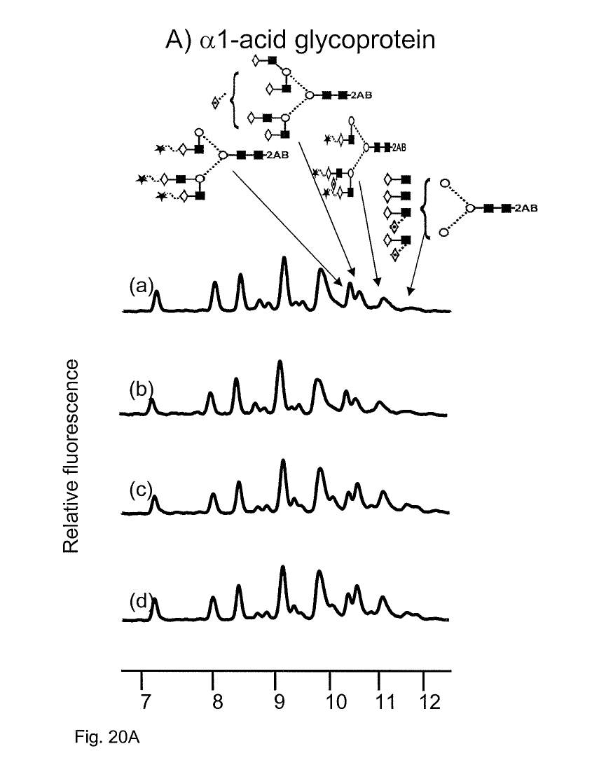

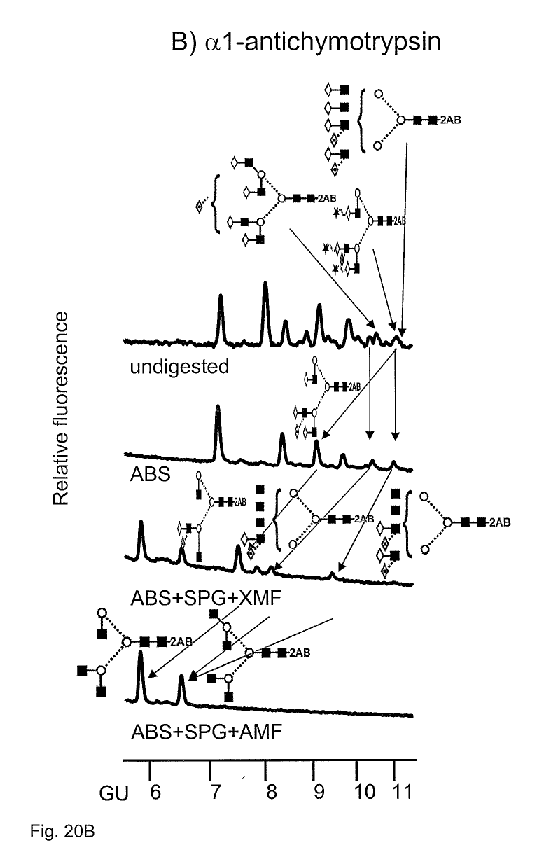

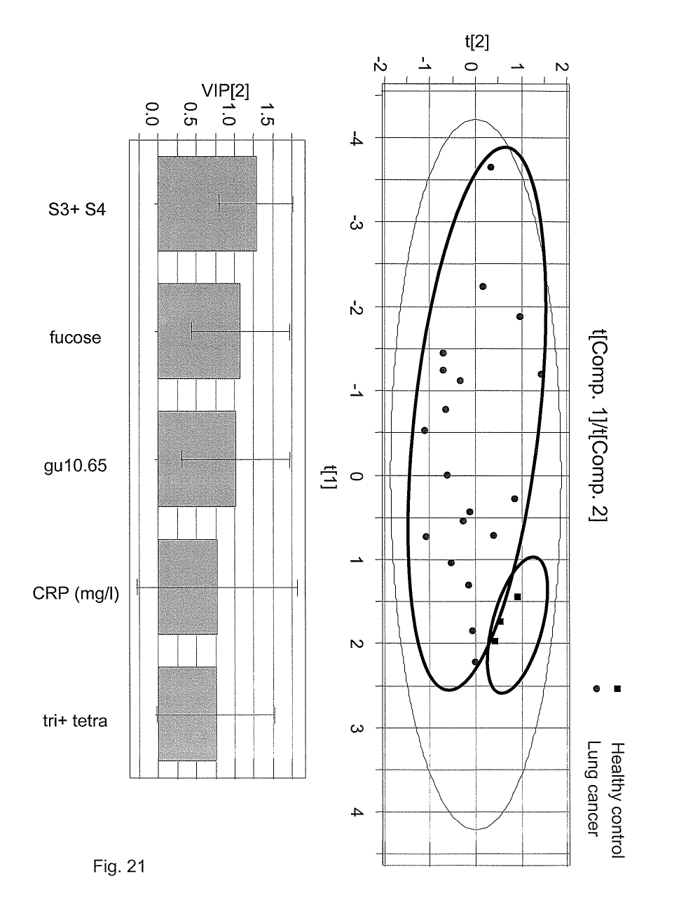

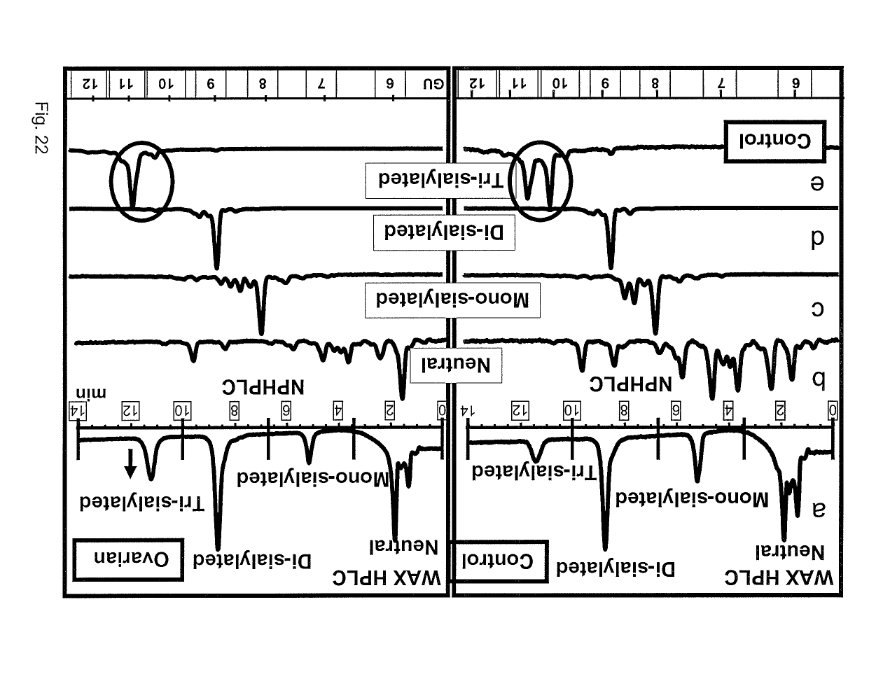

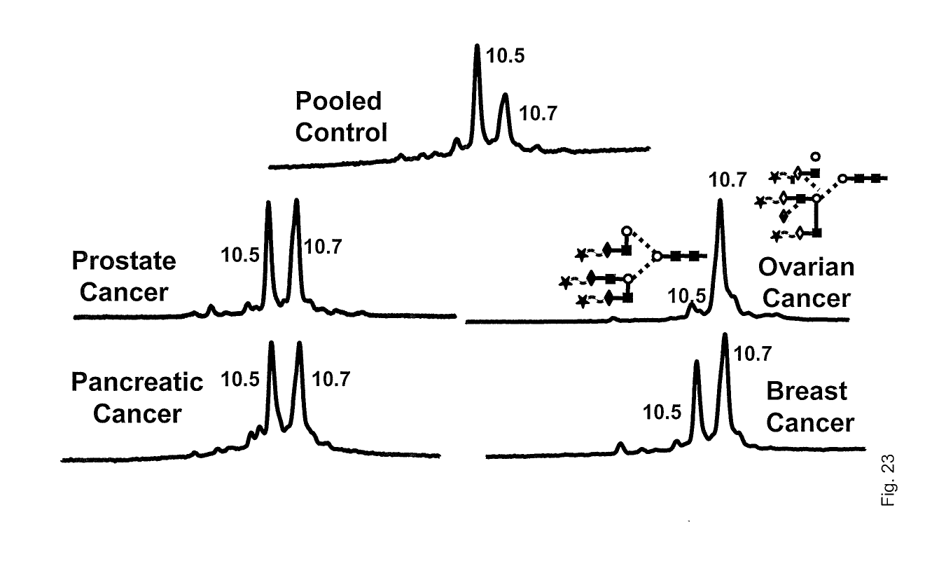

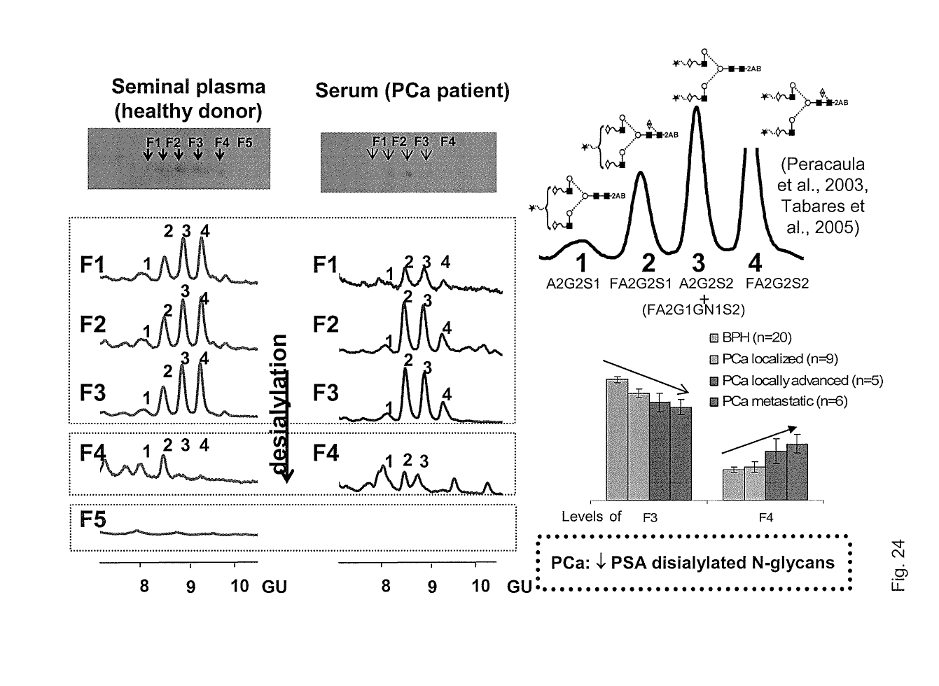

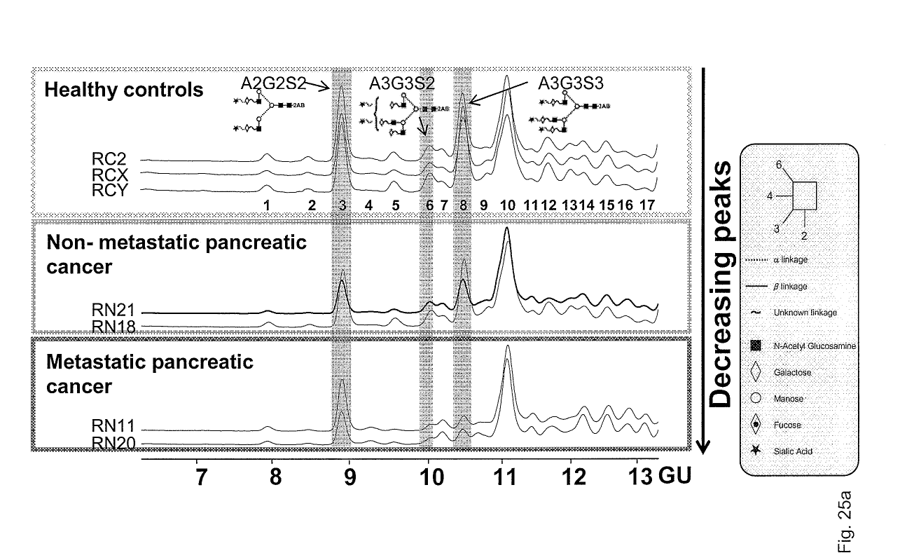

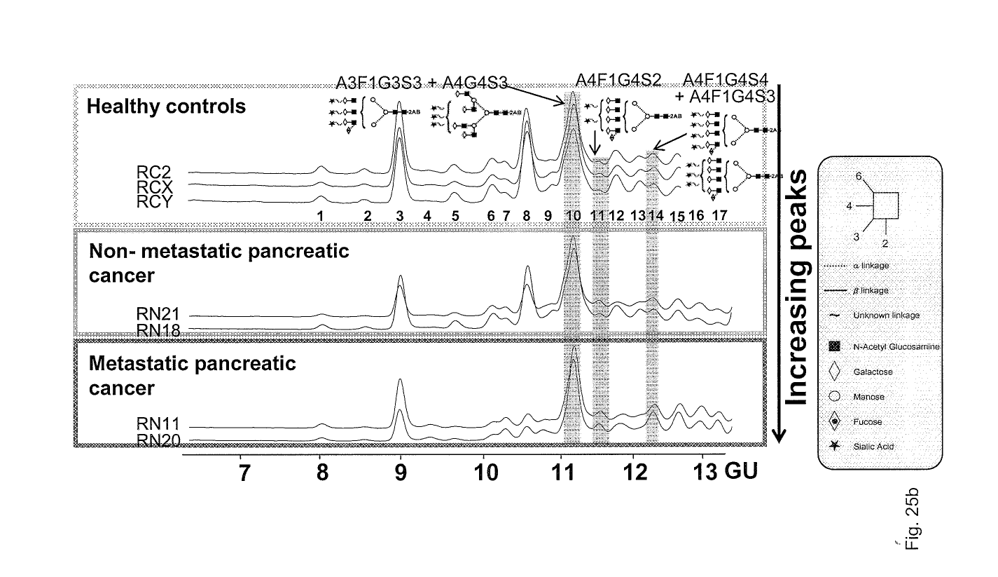

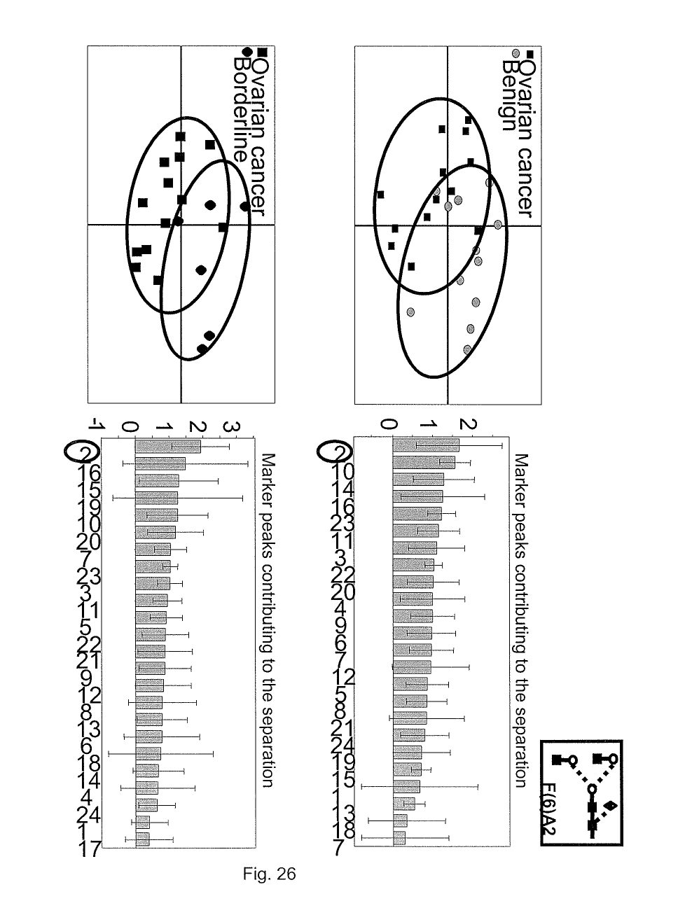

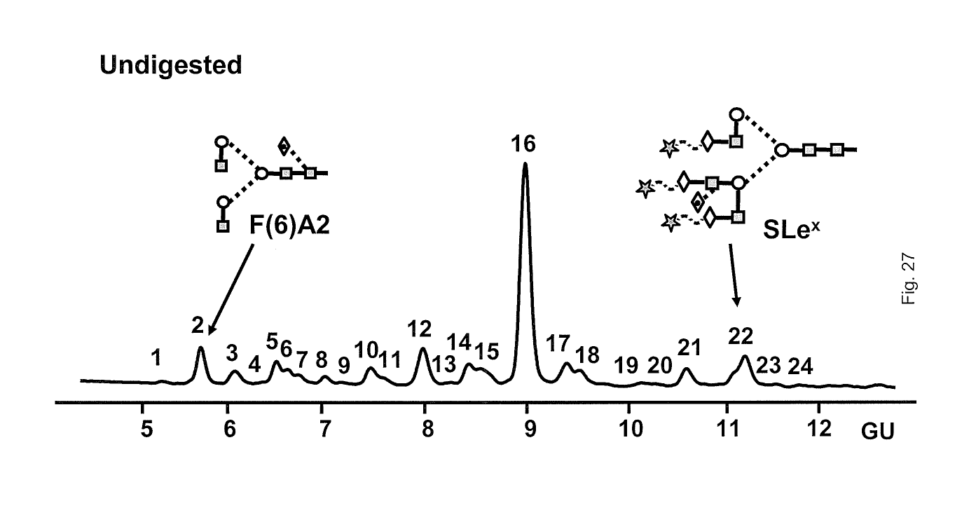

In the cases of haptoglobin β-chain, α1-acid glycoprotein and α1-antichymotrypsin, major glycosylation changes were identified (FIG. 19, 20). Haptoglobin was identified in the train of spots 1-6 with the highest protein score, except for complement C3 in spot 1 (Table 7). However, the N-linked glycosylation of complement C3 is known to consist of mannose structures, so the complex glycans detected over all these spots originated from haptoglobin, although traces of mannose have been detected too reflecting the co-migration of C3. α1-antichymotrypsin was identified in spot 8 with the highest protein score, although α1-antitrypsin was also found in this spot, but identified with lower score (Table 7) and with no glycans highlighted on α1-antichymotrypsin (unpublished data). Therefore, it also does not interfere with altered levels of glycans described on α1-antichymotrypsin (FIG. 20).

FIG. 19 shows the NPHPLC profiles of haptoglobin β-chain glycoforms from single spots in the train on 2D minigels of a control and stage III ovarian cancer patient B, FIG. 20 shows NPHPLC profiles of α1-acid glycoprotein 2D gel spots from pooled control, benign, malignant and metastatic sera and α1-antichymotrypsin from pooled malignant sample cut from a single 2D gel spot digested by exoglycosidases for structural assignment of the outer arm fucosylated structures.

The A3F1G3S3 on haptoglobin β-chain, α1-acid glycoprotein and a1-antichymotrypsin were identified. These changes in the relative proportions of glycoforms in the ovarian cancer patients' proteins contribute to the changes in the glycan profiles of whole serum, in particular to the neutral and tri-sialylated fraction of WAXHPLC. Similar profile changes were observed in all six haptoglobin β-chain spots and in an advanced ovarian cancer patient (FIG. 19), and pooled ovarian cancer patients sera comparing malignant and metastatic sera to benign and control sera (unpublished data). It has been demonstrated that the different spots contained different subsets of glycoforms. With increased acidity, the glycoform migrated further to the left on the gel (FIG. 18). In haptoglobin β-chain, the level of A3F1G3S3 is highest and A2G2S1 lowest in the most acidic glycoform (FIG. 19).

Discussion

The aim of this study was to identify which proteins were contributing to changes in the serum glycome of ovarian cancer patients and to determine whether changes in glycans of serum proteins could have potential utility as markers in ovarian cancer. In an initial pilot study analysing total serum N-glycans using quantitative and detailed normal phase (NP)HPLC, weak anion exchange (WAX) HPLC and mass spectrometry (MS), samples from three patients with advanced ovarian cancer were compared to a pooled control sample. Based on the findings high-throughput technology was used to monitor A3FG1 and FA2 (core fucosylated agalactosylated biantennary glycan structure) levels in a total of 90 samples from healthy controls, patients with ovarian cancer, benign gynaecological conditions or other gynaecological cancers. This confirmed the initial findings of increased expression in patients with ovarian cancer compared with controls or benign conditions and also in other cancers. Further investigations using techniques to determine the glycosylation status of proteins isolated from individual spots on fluorescently stained 2D-SDS PAGE gels found major and variable differences in glycoforms of several acute-phase proteins including haptoglobin, α1-acid glycoprotein, α1-antichymotrypsin and also in IgG.

Changes in Glycan Structures in Ovarian Cancer Serum Samples

Several glycosylation changes in advanced ovarian cancer patient serum samples have been observed. The most significant were increased levels of A3FG1 and FA2.

Increased levels of SLe^{x} in the tri-sialylated fraction suggest a change in regulation of fucosyltransferases in the liver hepatocytes. To result in SLe^{x} structures, the precursor core structure has to be sialylated first and then fucosylated by a (1,3/1,4) fucosyltransferases. Increased levels of SLe^{x} have been correlated to decreased expression of α1,2 fucosyltransferase, which competes with α2,3 sialyltransferase for the same substrate and increased expression of a (1,3/1,4) fucosyltransferases in human pancreatic cancer cells. The levels of A3FG1 in different stages of ovarian and other gynaecological cancers were determined and compared to benign gynaecological conditions. It was demonstrated that, although higher than controls, they are not specific for ovarian cancer. Increased levels of A3FG1 have also been found in inflammatory conditions of pancreatitis and sepsis.

Significant increases both in branching and sialylation were identified. Increased branching creates more sites for terminal sialic acid residues and together with sialyltransferase upregulation increases the sialylation. It correlates with advanced stage, tumour progression and metastasis. Changes in branching and increased sialylation have previously been identified in chronic inflammatory conditions. These changes reflect differences in expression levels of sialyltransferase and fucosyltranferases in the Golgi.

In addition to the increase in overall sialylation, a shift in sialic acid linkage from α2,3 to α2,6 in the disialylated fractions was also observed. These findings are in agreement with previous findings of decreased mRNA expression of α2,3 sialyltransferases responsible for N-linked glycosylation and increased α2,6 sialyltransferase in tumour tissues of ovarian cancer patients. This may suggest that the cytokines to which the tumour has been exposed have caused a similar shift in the glycoform populations on the tumour cells as we have been identified here in the serum. It is possible that the cytokines secreted at sites of inflammation (the tumour) find their way into the serum and affect the glycosylation machinery of the liver hepatocytes, to cause shifts in the serum glycoforms.

Another striking difference in ovarian cancer serum when compared with control serum is the doubling in the levels of FA2. This structure has previously been shown to be attached predominantly to IgG.

The major N-glycans attached to CA125 have been described as mostly mono-fucosylated biantennary, triantennary, and tetra-antennary bisected structures with no more than one sialic acid. Comparing the CA125 glycans with our major glycans level changes we propose that elevated levels of CA125 do not contribute to the major changes in whole serum glycans. The glycosylation changes may relate to specific glycoforms of particular glycoproteins in serum. CA125 is also elevated in chronic pancreatitis but not in sepsis.

Interestingly no change in glycosylation of serum glycans was observed in the examined malignant melanoma samples, where inflammation is not involved (FIG. 16).

Identification of Serum Glycoproteins Containing the Altered Levels of Glycans

Acute-Phase Response

The acute-phase response, which occurs when infection, trauma, surgery, burns or inflammatory conditions, leads to substantial changes in the plasma concentration of acute-phase proteins as a result of increased release of inflammatory cytokines such as IL-6 and TNF stimulate the increased production of C-reactive protein, serum amyloid A, haptoglobin, α1-acid glycoprotein, α1-antitrypsin, α1-antichymotrypsin and fibrinogen (positive acute-phase proteins) along with decreased levels of albumin and transferrin (negative acute-phase proteins). Using sensitive quantitative techniques in a pilot study, altered glycosylation on haptoglobin, α1-acid glycoprotein and α1-antichymotrypsin have been identified in advanced ovarian cancer patient sera.

Increase of Positive Acute-Phase Proteins in Plasma Correlates with Altered Glycosylation

Haptoglobin is a liver protein secreted into plasma which binds free haemoglobin in the plasma and makes it accessible to degradative enzymes. Haptoglobin β-chain expression increases in ovarian cancer, decreases with chemotherapy and correlates with CA125 levels. This increase in protein levels could account for some of the changes in the serum glycome. However, the results (FIG. 19) from the 2D gel analysis also show an increase in the SLe^{x} structure on the haptoglobin β-chain. This is consistent with results by Thompson et al. who identified an increased fucose content of haptoglobin which increased with tumour size. It has also been found that the SLe^{x} structure elevated on α1-acid glycoprotein and α1-antichymotrypsin (FIG. 20). They are both produced by the liver and secreted in plasma. SLe^{x} is also expressed during inflammation on all these proteins. α1-acid glycoprotein modulates the immune response during the acute-phase reaction. Its synthesis is controlled by glucocorticoids, interleukin-1 (IL-1) and IL-6. α1-antichymotrypsin can inhibit neutrophil cathepsin G and mast cell chymase, both of which can convert angiotensin-1 to the active angiotensin-2.

The increased levels of SLe^{x} structure on the haptoglobin β-chain, α1-antichymotrypsin and α1-acid glycoprotein in cancer and inflammation suggests that these glycosylation changes may contribute to increased concentrations of these acute-phase proteins concentrations. The addition of terminal sialic acid and fucose, inhibits the amount of free galactose accessible to the asialoglycoprotein receptor in liver and, as such, prolongs their clearance from the circulation resulting in their higher concentrations. The reason for increased concentrations of these glycoproteins could be for their anti-apoptotic and anti-inflammatory properties. These have been reported in the case of α1-acid glycoprotein and α1-antichymotrypsin. Their antiapoptotic properties may be beneficial to cancer progression.

Glycosylation of these liver proteins in serum may derive from the glycosylation process during their biosynthesis in the parenchymal cells of the liver; inflammatory cytokines, corticosteroids and growth factors appear to regulate these changes. Interestingly, only proteins that normally put on SLe^{x} have increased levels of this marker. Proteins which don't express SLe^{x} don't add it on in ovarian cancer e.g. transferrin.

Decreased Galactosylation on Immunoglobulin G has Impact on its Function

The N-linked analysis of the glycans on IgG from the ovarian cancer patients showed a significant decrease in the level of galactosylation and sialylation (FIG. 17 and Table β). Increase of agalactosyl IgG oligosaccharides can be result of decreased Gal-T activity in plasma cells, or increased production of specific subsets of plasma cells with low expression levels of galactosyltransferases. Different glycoforms may differ in efficiency of interaction with ligands. The IgG-G0 glycoform is elevated in rheumatoid arthritis serum and terminal GlcNAc of this glycoform on the Fc region of the IgG molecule clustered, for example on synovial tissue, can be recognized by collagenous lectin mannose-binding protein (MBL) resulting in complement activation. It has also been shown that sialylation of IgG reduces cytotoxicity of natural killer cells, exhibiting anti-inflammatory effect. Increase of agalactosyl IgG glycoform has predominantly been identified with tumour progression and metastasis of gastric and lung cancer (Kanoh et al. 2004), as well as in other diseases such as rheumatoid arthritis, tuberculosis, inflammatory bowel disease (Parekh et al. 1985; Axford et al. 1992) and vasculitis (Holland et al. 2002).

Therefore this increase of agalactosylated glycans on IgG of ovarian cancer sera may be indicative of an inflammatory state.

In conclusion, newly developed high throughput techniques enable rapid monitoring of glycosylation changes in serum. Differences between control and advanced ovarian cancer sera have been described including a doubling in the amount of FA2 and SLe^{x} structures in whole serum glycan profiles and a shift in the sialic acid linkage from α2,3 to α2,6 in disialylated fractions. It has been demonstrated that the level of A3FG1 alone is not specific for ovarian cancer, but a combination of FA2 and A3FG1 significantly improves separation of benign gynaecological conditions from ovarian cancer. To investigate further which protein glycans contribute to these changes in total serum glycans, serum glycoproteins carrying these glycans were identified. Newly developed sensitive HPLC based technology enabled screening of all proteins from the same patient. This analysis of the glycosylation of protein excised from single spots on a 2D minigel show: haptoglobin β-chain, α1-acid glycoprotein and α1-antichymotrypsin with elevated SLe^{x} structure and IgG with decreased galactosylation and sialylation. Only proteins with SLe^{x} have increased levels of this epitope. All these glycosylation changes suggest that cancer mimics chronic inflammation. This theory is supported by glycosylation described in inflammatory conditions sepsis and acute pancreatitis where many of these glycosylation changes have also been observed, and the fact, that in our malignant melanoma samples, where no inflammation is involved, there were no alterations in glycan levels. Cancer, especially in the late stages, can cause chronic inflammation. The inflammation results in an acute-phase response, in which the liver produces acute-phase proteins which have also anti-apoptotic properties. In inflammation it helps to reconstitute the damaged tissue but it protects and promotes cancer cells considering them for its own. If this hypothesis is correct, anti-inflammatory drugs should be powerful in cancer treatment. Non-steroidal anti-inflammatory drugs (NSAID) are efficacious both in preventing and protecting against cancer development and progression.

Summary

Performing glycosylation analysis on whole, i.e. not depleted and not purified, samples can be particularly beneficial for cancer diagnostics and monitoring. Although differences in the glycosylation profile can be associated with the presence in samples of cancer patients of glycoproteins specifically associated with cancer, such as alpha-fetoprotein many other tumour glycoproteins, i.e. glycoproteins that are not specific inflammatory markers of cancer, can be expected to carry altered glycosylation because glycosylation pathways are usually disturbed in tumour cells. Based on the above, performing detailed glycosylation analysis on samples of whole body fluid or body tissue, without isolating or purifying specific glycoproteins, can be expected to identify glycosylation markers of cancer amplified compared with glycosylation analysis of purified glycoproteins.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A method for the diagnosis of a cancerous and/or malignant condition, the prognosis of a cancerous and/or malignant condition, and/or the monitoring of a response to treatment of a cancerous and/or malignant condition in a subject, the method comprising the steps of:—
providing a test sample from the subject,
determining a level in the test sample of two or more glycosylation markers for the cancerous and/or malignant condition,
providing a diagnosis, prognosis or determination of the response based on the level of the two or more glycosylation markers.

2. A method as claimed in claim 1, wherein the level of 2, 3, 4, 5, 6, 7, 8, 9 or 10 glycosylation markers for the cancerous and/or malignant condition are determined.

3. A method for the diagnosis of a cancerous and/or malignant condition, the prognosis of a cancerous and/or malignant condition and/or the monitoring of a response to treatment of a cancerous and/or malignant condition in a subject, the method comprising the steps of:
providing a test sample from the subject,
determining the level in the test sample of one or more glycosylation marker(s) of the cancerous and/or malignant condition and one or more non-glycosylation marker(s) of the cancerous and/or malignant condition, and
providing a diagnosis, prognosis or determination of the response based on the level of the one or more glycosylation markers and the one or more non-glycosylation markers.

4. A method as claimed in claim 3, wherein the level of 2, 3, 4, 5, 6, 7, 8, 9 or 10 glycosylation markers for the cancerous and/or malignant condition are determined.

5. A method as claimed in claim 3, wherein the level of 2, 3, 4, 5, 6, 7, 8, 9 or 10 non-glycosylation markers for the cancerous and/or malignant condition are determined.

6. A method as claimed in claim 3, wherein the non-glycosylation markers are selected from the group consisting of inflammatory markers, cytokines, chemokines, genetic markers, Catecholamines, Immunoglobulins, markers for angiogenesis, or the like, or any combination thereof.

7. A method as claimed in claim 3, wherein the non-glycosylation markers are selected from the group consisting of Alphafetoprotein, NMP22, Carcinoembryonic antigen (CEA), HER-2, CA 15-3, CA 27-29, CA 125, CA 19-9, and C-reactive protein (CRP), IL-4, IL-10, IL-1α and IL-1β, MCP-1, or the like, or any combination thereof.

8. A method for the diagnosis of a cancerous and/or malignant condition, the prognosis of a cancerous and/or malignant condition and/or the monitoring of a response to treatment of a cancerous and/or malignant condition in a subject, the method comprising the steps of:
providing a test sample from the subject,
determining the level of at least one marker selected from the group comprising glycans with GU values greater than 10.65, SLe^{x} structures, A2FG1 derived from digestion of SLe^{x}, A3FG1 derived from digestion of SLe^{x}, A4FG1 derived from digestion of SLe^{x}, sialylated tri-antennary glycans, sialylated tetra-antennary glycans, glycans containing α1,3 fucose, α1,3 monofucosylated tri-antennary glycans, α1,3 difucosylated tri-antennary glycans, α1,3 monofucosylated tetra-antennary glycans, α1,3 difucosylated tetra-antennary glycans, tetra-antennary glycans with lactosamine extensions, ratio of α2,3 sialylated glycans to α2,6 sialylated glycans, agalactosylated fucosylated biantennary glycans, core fucosylated agalactosylated biantennary glycans, core fucosylated monosialylated glycans on transferrin, SLe^{x} on glycans on haptoglobin β-chain, A3FG1 derived from digestion of SLe^{x} on glycans on haptoglobin β-chain, A4FG1 derived from digestion of SLe^{x} on glycans on haptoglobin β-chain, SLe^{x} on glycans on α1-acid glycoprotein, A3FG1 derived from digestion of SLe^{x} on glycans on α1-acid glycoprotein, SLe^{x} on glycans on α1-antichymotrypsin, A3FG1 derived from digestion of SLe^{x} on glycans on α1-antichymotrypsin, tetra-antennary tetragalactosylated glycans on α1-antitrypsin, core fucosylated agalactosylated biantennary glycans on IgG, agalactosylated glycans on IgG, sialylation on glycans on IgG, galactosylation on glycans on IgG, FA2G2S1 on glycans on transferrin, FA2BG2S1 on glycans on transferrin and A4G4 glycans on α1-antitrypsin, or the like, or any combination thereof, and
providing a diagnosis, prognosis or determination of the response based on the determined level of the at least one marker.

9. A method as claimed in claim 1, wherein the glycosylation markers are selected from the group consisting of changes in glycan branching; changes in levels of oligomannose, of hybrid and complex type N-glycans, of O-glycans, or of components thereof; changes in ratios of levels between glycans, GU values; or the like; or any combination thereof.

10. A method as claimed in claim 1,
wherein the glycosylation markers are selected from the group consisting of: glycans with GU values greater than 10.65, SLe^{x} structures, A2FG1 derived from digestion of SLe^{x}, A3FG1 derived from digestion of SLe^{x}, A4FG1 derived from digestion of SLe^{x}, sialylated tri-antennary, sialylated tetra-antennary glycans, glycans containing α1,3 fucose, α1,3 monofucosylated tri-antennary glycans, α1,3 difucosylated tri-antennary glycans, α1,3 monofucosylated tetra-antennary glycans, α1,3 difucosylated tetra-antennary glycans, tetra-antennary glycans with lactosamine extensions, ratio of α2,3 sialylated glycans to α2,6 sialylated glycans, agalactosylated fucosylated biantennary glycans, core fucosylated agalactosylated biantennary glycans, core fucosylated monosialylated glycans on transferrin, SLe^{x} on glycans on haptoglobin β-chain, A3FG1 derived from digestion of SLe^{x} on glycans on haptoglobin β-chain, A4FG1 derived from digestion of SLe^{x} on glycans on haptoglobin β-chain, SLe^{x} on glycans on α1-acid glycoprotein, A3FG1 derived from digestion of SLe^{x} on glycans on α1-acid glycoprotein, SLe^{x} on glycans on α1-antichymotrypsin, A3FG1 derived from digestion of SLe^{x} on glycans on α1-antichymotrypsin, tetra-antennary tetragalactosylated glycans on α1-antitrypsin, core fucosylated agalactosylated biantennary glycans on IgG, agalactosylated glycans on IgG, sialylation on glycans on IgG, galactosylation on glycans on IgG, FA2G2S1 on glycans on transferrin, FA2BG2S1 on glycans on transferrin and A4G4 on glycans on α1-antitrypsin, or the like, or any combination thereof.

11. The method as claimed in claim 1, wherein the method involves the analysis of all members of one of the following groups of glycosylation markers S3 and S4, fucose, GU of 10.65 and tri and tetra antennary glycans; A3FG1 and FA2; SLe^{x} and fucosylated agalactosylated biantennary glycans; or the like; or combinations thereof.

12. A method as claimed in claim 3, wherein the method involves the analysis of all members of one of the following groups of markers CRP and any one, two, three, four, five, six, or more of any glycosylation marker(s); CRP and any of one, two or three of the glycosylation markers S3 and S4, fucose, GU of 10.65, tri and tetra-antennary glycans; S3 and S4, fucose, GU of 10.65, tri and tetra-antennary glycans, and CRP; fucosylated agalactosylated biantennary glycans and CRP; one or more pro-inflammatory cytokines and one or more glycosylation marker; one or more anti-inflammatory cytokine and one or more glycosylation marker; one or more chemokine and one or more glycosylation marker; or the like; or combinations thereof.

13. The method as claimed in claim 1, wherein the subject is a human.

14. The method as claimed in claim 1, wherein the test sample comprises a body fluid or body tissue.

15. The method as claimed in claim 14, wherein the body fluid comprises serum, plasma or urine.

16. The method as claimed in claim 1, comprising comparing the level of the one or more markers in the test sample with a level of the one or more markers in a control sample to determine the diagnosis, prognosis, and/or response.

17. The method as claimed in claim 16, wherein an increase in the level of the one or more markers in the test sample as compared to the control sample indicates the presence of cancer.

18. The method as claimed in claim 1, wherein determining the level in the test sample of the two or more markers comprises determining the level of the two or more markers on one or more acute phase proteins in the test sample.

19. The method of claim 18, wherein the one or more acute phase proteins are selected from the group consisting of: serum amyloid A, haptoglobin, α1-acid glycoprotein, α1-antitrypsin, α1-antichymotrypsin, fibrinogen and transferrin.

20. The method of claim 18, the method comprising isolating the one or more acute phase proteins from the test sample prior to determining the level of the two or more markers on the one or more acute phase proteins.

21. The method as claimed in claim 1, wherein determining the level in the test sample of the two or more markers comprises releasing a pool of glycans from total glycoproteins in the test sample and determining the level of the two or more markers in the pool of glycans.

22. The method as claimed in claim 1, wherein determining the level in the test sample of the two or more markers comprises performing chromatography on the sample or a derivative or component thereof.

23. The method as claimed in claim 1, wherein determining the level in the test sample of the two or more markers comprises performing mass spectrometry, immuno-PCR, two-dimensional gel electrophoresis, ELISA, lectin ELISA, Western blot, immunoassay, lectin immunoassay, or one dimensional gel electrophoresis on the sample or a derivative or component thereof.

24. A method for assessing the inflammatory state of a subject, the method comprising the steps of:—
providing a test sample from a subject,
determining a level in the test sample of two or more glycosylation markers for chronic inflammation,
providing an assessment based on the level of the two or more glycosylation marker.

25. A method for assessing the inflammatory state of a subject, the method comprising the steps of:
providing a test sample from a subject,
determining the level in the test sample of one or more glycosylation marker(s) of chronic inflammation and one or more non-glycosylation marker(s) of chronic inflammation, and
providing an assessment based on the level of the one or more glycosylation markers and the one or more non-glycosylation markers.

26. A method for assessing the inflammatory state of a subject, the method comprising the steps of:
providing a test sample from a subject,
determining the level of at least one marker selected from the group comprising glycans with GU values greater than 10.65, SLe^{x} structures, A2FG1 derived from digestion of SLe^{x}, A3FG1 derived from digestion of SLe^{x}, A4FG1 derived from digestion of SLe^{x}, sialylated tri-antennary glycans, sialylated tetra-antennary glycans, glycans containing α1,3 fucose, α1,3 monofucosylated tri-antennary glycans, α1,3 difucosylated tri-antennary glycans, α1,3 monofucosylated tetra-antennary glycans, α1,3 difucosylated tetra-antennary glycans, tetra-antennary glycans with lactosamine extensions, ratio of α2,3 sialylated glycans to α2,6 sialylated glycans, agalactosylated fucosylated biantennary glycans, core fucosylated agalactosylated biantennary glycans, core fucosylated monosialylated glycans on transferrin, SLe^{x} on glycans on haptoglobin β-chain, A3FG1 derived from digestion of SLe^{x} on glycans on haptoglobin β-chain, A4FG1 derived from digestion of SLe^{x} on glycans on haptoglobin β-chain, SLe^{x} on glycans on α1-acid glycoprotein, A3FG1 derived from digestion of SLe^{x} on glycans on α1-acid glycoprotein, SLe^{x} on glycans on α1-antichymotrypsin, A3FG1 derived from digestion of SLe^{x} on glycans on α1-antichymotrypsin, tetra-antennary tetragalactosylated glycans on α1-antitrypsin, core fucosylated agalactosylated biantennary glycans on IgG, agalactosylated glycans on IgG, sialylation on glycans on IgG, galactosylation on glycans on IgG, FA2G2S1 on glycans on transferrin, FA2BG2S1 on glycans on transferrin and A4G4 glycans on α1-antitrypsin, or the like, or any combination thereof, and
providing an assessment based on the determined level of the at least one marker.
